(19) United States
(12) Patent Application Publication
(10) Pub. No.: US 2019/0183927 A1
(43) Pub. Date: Jun. 20, 2019

(54) MEDICAL AND NUTRITIONAL COMPOSITIONS AND METHODS OF USE

(71) Applicant: Sulfilatec, Inc., Huntsville, AL (US)

(72) Inventors: Christopher Cummings, Madison, AL (US); David Shifrin, Nashville, TN (US)

(21) Appl. No.: 16/324,697

(22) PCT Filed: Aug. 11, 2017
(86) PCT No.: PCT/US2017/046570

§ 371 Date: Feb. 11, 2019

Related U.S. Application Data
(60) Provisional application No. 62/373,656, filed on Aug. 11, 2016.
(60) Provisional application No. 62/402,291, filed on Sep. 30, 2016.
(60) Provisional application No. 62/402,309, filed on Sep. 30, 2016.
(60) Provisional application No. 62/424,057, filed on Nov. 18, 2016.
(60) Provisional application No. 62/458,681, filed on Feb. 14, 2017.

(51) Int. Cl. A61K 33/14; A61K 33/00; A61K 45/06; A61K 31/145; A01N 1/02; A23L 27/40; A23L 33/16

(52) U.S. Cl. CPC A61K 33/14; A61K 33/00; A61K 45/06; A23V 2002/00; A01N 1/021; A23L 27/40; A23L 33/16; A61K 31/145

(57) ABSTRACT

Embodiments of the present invention are directed to improved medical saline compositions and methods of use thereof. Other embodiments of the invention concern nutritional compositions and methods that promote the health of the heart, kidneys, and vasculature in a subject at risk of developing disease.

This application claims priority from U.S. Provisional Application No. 62/373,656 filed on Aug. 11, 2016, U.S. Provisional Application No. 62/402,291 filed on Sep. 30, 2016, U.S. Provisional Application No. 62/402,309 filed on Sep. 30, 2016, and U.S. Provisional Application No. 62/424,057, filed on Nov. 18, 2016, and U.S. Provisional Application No. 62/458,681, filed on Feb. 14, 2017, the entire contents of each which are incorporated herein by reference.

### FIELD OF THE INVENTION

The invention concerns compositions and methods for use in the field of medicine. Specifically, the invention provides medical saline compositions and nutritional compositions and methods for their use in a subject.

### BACKGROUND OF THE INVENTION

Cardiovascular disease is a leading cause of mortality in the United States of America. Kidney disease, including chronic kidney disease (CKD) and end-stage renal disease (ESRD), are increasingly prevalent and severely damage the quality of life for patients. Importantly, many forms of cardiovascular disease and kidney disease are chronic diseases that require long-term management. There are acute forms of these diseases as well, such as but not limited to acute kidney injury and acute heart failure. The financial burden imposed by these diseases is enormous. In the United States, 2013 Medicare expenditures for patients with either CKD or ESRD exceeded $80 billion (2015 USRDS Annual Report). The prevalence of these diseases is also quite large. It has been estimated that over 100 million individuals in the United Sates have some form of cardiovascular disease and/or kidney disease. These diseases represent the clinical manifestations of poor health in the heart, kidneys, and/or vasculature of patients.

0.9% NaCl, also simply termed 0.9% saline, is used to irrigate wounds, wash medical devices, and administer pharmaceutical drugs to subjects, among other purposes. Other saline formulations, such as Ringer's solution, hypertonic saline, and hypotonic saline, can contain higher or lower amounts of NaCl and/or the ingredients bicarbonate, potassium, lactate, and calcium.

### SUMMARY OF THE INVENTION

Embodiments of the invention comprise a pharmaceutical composition that comprises an amount of Br⁻ and Cl⁻ ions, wherein the molar amount of Br⁻ ions is between about 0.0338% and about 4.613% of the molar amount of Cl⁻. For example, some embodiments comprise an amount of Br⁻ that is 0.0338%, 0.04%, 0.05%, 0.06%, 0.07%, 0.08%, 0.09%, 0.10%, 0.12%, 0.14%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, 1.00%, 1.02%, 1.04%, 1.06%, 1.08%, 1.10%, 1.12%, 1.14%, 1.16%, 1.18%, 1.20%, 1.22%, 1.24%, 1.26%, 1.28%, 1.30%, 1.32%, 1.34%, 1.36%, 1.38%, 1.40%, 1.42%, 1.44%, 1.46%, 1.48%, 1.50%, 1.52%, 1.54%, 1.56%, 1.58%, 1.60%, 1.62%, 1.64%, 1.66%, 1.68%, 1.70%, 1.72%, 1.74%, 1.76%, 1.78%, 1.80%, 1.82%, 1.84%, 1.86%, 1.88%, 1.90%, 1.92%, 1.94%, 1.96%, 1.98%, 2.00%, 2.02%, 2.04%, 2.06%, 2.08%, 2.10%, 2.12%, 2.14%, 2.16%, 2.18%, 2.20%, 2.22%, 2.24%, 2.26%, 2.28%, 2.30%, 2.32%, 2.34%, 2.36%, 2.38%, 2.40%, 2.42%, 2.44%, 2.46%, 2.48%, 2.50%, 2.52%, 2.54%, 2.56%, 2.58%, 2.60%, 2.62%, 2.64%, 2.66%, 2.68%, 2.70%, 2.72%, 2.74%, 2.76%, 2.78%, 2.80%, 2.82%, 2.84%, 2.86%, 2.88%, 2.90%, 2.92%, 2.94%, 2.96%, 2.98%, 3.00%, 3.02%, 3.04%, 3.06%, 3.08%, 3.10%, 3.12%, 3.14%, 3.16%, 3.18%, 3.20%, 3.22%, 3.24%, 3.26%, 3.28%, 3.30%, 3.32%, 3.34%, 3.36%, 3.38%, 3.40%, 3.42%, 3.44%, 3.46%, 3.48%, 3.50%, 3.52%, 3.54%, 3.56%, 3.58%, 3.60%, 3.62%, 3.64%, 3.66%, 3.68%, 3.70%, 3.72%, 3.74%, 3.76%, 3.78%, 3.80%, 3.82%, 3.84%, 3.86%, 3.88%, 3.90%, 3.92%, 3.94%, 3.96%, 3.98%, 4.00%, 4.02%, 4.04%, 4.06%, 4.08%, 4.10%, 4.12%, 4.14%, 4.16%, 4.18%, 4.20%, 4.22%, 4.24%, 4.26%, 4.28%, 4.30%, 4.32%, 4.34%, 4.36%, 4.38%, 4.40%, 4.42%, 4.44%, 4.46%, 4.48%, 4.50%, 4.52%, 4.54%, 4.56%, 4.58%, 4.60%, 4.613%, or any other amount that is between 0.0338% and 4.613% of the amount of Cl⁻ on a molar basis. In some embodiments, the Br⁻ and Cl⁻ ions are formulated as any pharmaceutically acceptable salts. In some embodiments, the Br⁻ and Cl⁻ ions are formulated as pharmaceutically acceptable salts of Na⁺, K⁺, Mg²⁺, Ca²⁺, or any combination thereof. In some embodiments, the composition is formulated as a pill, patch, strip, or liquid. In some embodiments, the composition is administered to a subject via oral, transdermal, injection, rectal, inhalation, nasal, or a combination thereof, routes of administration.

Embodiments of the invention comprise a method of treating or preventing disease in a subject, the method comprising administering a therapeutically effective amount of a composition comprising an amount of Br⁻ and Cl⁻ ions wherein the molar amount of Br⁻ ions that between about 0.0338% and about 4.613% of the molar amount of Cl⁻. In some embodiments, the composition is a pharmaceutical composition. In some embodiments, the disease comprises cancer, cardiovascular disease, kidney disease, end stage renal disease, an eye disease, cystic fibrosis, a degenerative disease, a blood disorder, diabetes, a wound, a skin irritation, inflammation, an injury, a medical condition requiring dialysis treatment, or a combination thereof.

Embodiments of the invention comprise pharmaceutical compositions and methods for administering the same to a subject. Some embodiments of the invention are directed to a pharmaceutical composition comprising an amount between 30 μM and 1 mM of bromide ions (Br⁻) and an amount between 17.1 mM and 200 mM of chloride ions (Cl⁻). In some embodiments, the amount of Br⁻ is about 30 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 170 μM, 175 μM, 180 μM, 185 μM, 190 μM, 195 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1 mM, or any othe amount between about 30 μM and about 1 mM. In some embodiments, the amount of Cl⁻ is about 17.1 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, or any other amount between about 17.1 mM and about 200 mM. In some embodiments, the amount of Mg²⁺ is about 0.75 mM, 0.80 mM, 0.85 mM, 0.90 mM, 0.95 mM, 1.05 mM, 1.05 mM, 1.10 mM, 1.15 mM, 1.20 mM, 1.25 mM, 1.30 mM, 1.35 mM, 1.40 mM, 1.45 mM, 1.50 mM, 1.55 mM, 1.60 mM, 1.65 mM, 1.70 mM, 1.75 mM, 1.80 mM, 1.85 mM, 1.90 mM, 1.95 mM, 2.00 mM, 2.05 mM, 2.10 mM, 2.15 mM, 2.20 mM, 2.25 mM, 2.30 mM, 2.35 mM, 2.40 mM, 2.45 mM, 2.50 mM, 2.55 mM, 2.60 mM, 2.65 mM, 2.70 mM, 2.75 mM, 2.80 mM, 2.85 mM, 2.90 mM, 2.95 mM, 3.0 mM, or any other amount between about 0.75 mM and about 3.0 mM. In some embodiments, the composition comprises an amount between 50 μM and 350 μM of bromide (Br⁻). In some embodiments, the composition comprises an amount of 0.9% sodium chloride (NaCl). Embodiments of the invention can use sodium bromide, potassium bromide, magnesium bromide, or a combination thereof as the source of Br⁻. In some embodiments, the composition is manufactured using pharmaceutical grade sodium bromide, potassium bromide, or magnesium bromide. In some embodiments, the composition is administered to a subject. In some embodiments, the composition is used as a drug or as a medical device. In some embodiments, the composition is used as a replacement solution for renal replacement therapy. In some embodiments, the subject comprises a human or animal. In some embodiments, the subject comprises a human, dog, cat, mouse, rabbit, rat, hamster, guinea pig, horse, cow, goat, sheep, pig, chicken, or turkey.

In some embodiments, compositions of the invention comprise bromide ions and sodium chloride. In certain embodiments, compositions of the invention comprise about 0.9% sodium chloride. In some embodiments, compositions of the invention comprise more than about 0.9% sodium chloride. In other embodiments, compositions of the invention comprise less than about 0.9% sodium chloride but more than about 0.1% sodium chloride. In some embodiments, compositions of the invention comprise about 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 2.0%, 3.0%, 3.5%, 4.0%, 4.5%, 5.0%, 6.0%, 7.0%, 7.5%, 8.0%, 9.0%, 10.0%, 11%, 12%, 13%, 14%, 15%, 16%, 17%, 18%, 19%, 20%, or any other amount greater than about 0.1% sodium chloride. In some embodiments, the purity of the sodium chloride is at least of pharmaceutical grade or of United States Pharmacopeia (USP) grade.

In some embodiments, compositions of the invention comprise a source of chloride ions other than sodium chloride. Non-limiting examples of non-sodium chloride sources of chloride ions include magnesium chloride (MgCl₂), potassium chloride (KCl), calcium chloride (CaCl₂), or any pharmaceutically acceptable source of chloride. In some embodiments compositions of the invention comprise about 0.9% chloride. In some embodiments, compositions of the invention comprise more than about 0.9% chloride. In other embodiments, compositions of the invention comprise less than about 0.9% chloride but more than about 0.1% sodium chloride. In some embodiments, compositions of the invention comprise about 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 2.0%, 3.0%, 3.5%, 4.0%, 4.5%, 5.0%, 6.0%, 7.0%, 7.5%, 8.0%, 9.0%, 10.0%, 11%, 12%, 13%, 14%, 15%, 16%, 17%, 18%, 19%, 20%, or any other amount between about 0.1% and about 20.0% sodium chloride.

In some embodiments, compositions of the invention comprise an amount between about 30 mM and about 1 mM bromide ions (Br⁻), an amount between about 17.1 mM and about 200 mM chloride ions (Cl⁻), and a therapeutically effective amount of at least one of lactate, calcium, magnesium, bicarbonate, potassium, sodium, hydrogen phosphate, chloride, dextrose, or a combination thereof. For example, compositions of the invention can comprise Br⁻, Cl⁻, and at least one of the following:
About 0 mEq/L-about 35 mEq/L lactate,
About 1 mEq/L-about 5 mEq/L calcium,
About 0.5 mEq/L-about 3 mEq/L magnesium
About 22 mEq/L-about 32 mEq/L bicarbonate (HCO₃⁻),
About 0 mEq/L-about 4 mEq/L potassium,
About 140 mEq/L sodium,
About 0 mEq/L-about 1 mEq/L hydrogen phosphate (HPO₄²⁻),
About 100 mEq/L-about 130 mEq/L chloride,
About 0 mEq/L-about 100 mEq/L dextrose,
or a combination thereof.

In some embodiments, compositions of the invention are applied to a medical device. In some embodiments, compositions of the invention are used to cleanse, wash, moisten, hydrate, or lubricate the medical device. In some embodiments, compositions are co-administered with a medical device to a subject. In some embodiments, the subject has a tissue injury. Non-limiting examples of a tissue injury comprise a lesion, wound, burn, or ulcer. Non-limiting examples of the medical devices comprise a surgical device, a syringe, a needle, a suture, a contact lens, a medical device used in dialysis, a dialysis machine, a port, a fistula, a catheter, a graft, a machine used in the medical care of a subject, a component of a machine used in the medical care of a subject, a stent, a plug, a patch, an injection filler, a powder, a bandage, a gauze, a negative pressure device, a hydrophobic dressing, a hemostatic agent, an absorbent, a hydrating agent, an osmotic agent, a preservative, a wash, a lotion, a cream, a gel, a paste, an ointment, a rinse, an eye drop, an ear drop, a spray, a nasal spray, a matrix, a scaffold, an acellular scaffold, a collagen-based device, a camera, a pill camera, a tube, a connector, a needle-free connector, a closed system, transfer device, a hemodynamic monitoring system, a bag, a balloon, an intravenous line, a central line, an arterial line, or a combination thereof. In some embodiments, compositions of the invention are contacted with a medical device through a cleaning, stirring, soaking, shaking, rinsing, plunging, washing, hydrating, perfusing, or spraying action.

In some embodiments, compositions of the invention are applied to an organ, a tissue, or a tissue product. Non-limiting examples of organs, tissues, or tissue products comprise harvested tissues, engineered tissues, cultured tissues, human tissues, animal tissues, autologous tissues, bone, ligaments, tendons, vascularized organs, lung, kidney, heart, heart tissue, liver, pancreas, corneas, blood, stem cells, progenitor cells, skin, dura mater, oocytes, semen, and combinations thereof. In some embodiments, compositions of the invention are contacted with an organ, a tissue, or a tissue product through a cleaning, stirring, soaking, shaking, rinsing, plunging, washing, hydrating, perfusing, or spraying action.

In some embodiments, compositions of the invention are administered to a subject. Non-limiting examples of routes of administration comprise parenteral; oronasal; intranasal; intratracheal; topical, such as but not limited to irrigation, debridement, washing, hydration, or perfusion; injection; and intradermal; and others known to the art. For example, in some embodiments, compositions of the invention are administered to a subject via a topical, oral, or injection route of administration. In other embodiments, compositions of the invention are administered to a subject via a dialysis system. In some embodiments, the subject has sepsis, burn injury, trauma injury, fluid loss, dehydration, acid-base imbalance, electrolyte imbalance, kidney injury or disease, cancer, neurologic disease, diabetes, metabolic syndrome, cardiovascular disease, gastrointestinal disorder or disease, ear infection, congestion, or an eye disease. In some embodiments, the subject is undergoing or recovering from a surgery. In some embodiments, the subject is a human. In other embodiments, the subject is a non-human animal, such as but not limited to a dog, cat, mouse, rabbit, rat, hamster, guinea pig, horse, cow, goat, sheep, pig, chicken, or turkey.

In some embodiments, compositions of the invention are topically administered to a subject. In some embodiments, compositions of the invention are applied to intact, broken, cut, incised, wounded, burned, aged, diseased, or damaged skin of a subject. In some embodiments, compositions of the invention are used for irrigation, cleansing, hydrating, debriding, lubricating, or moistening purposes. In some embodiments, compositions of the invention are topically applied to an ulcer, with non-limiting examples including a chronic ulcer, a dermal ulcer, a pressure ulcer, a mouth sore, or a diabetic ulcer. In some embodiments, compositions of the invention are topically applied to a wound that is caused by a trauma, an aging process, a disease, or a surgical operation.

In some embodiments, compositions of the invention are administered from a dropper, syringe, needle, capsule, tube, aerosol can, inhaler, bottle, vial, or packet. In some embodiments, the dropper is an eye dropper. In some embodiments, the syringe is a pre-filled syringe.

In some embodiments, compositions of the invention are used in a hospital. In other embodiments, compositions are used in a medical clinic. In other embodiments, compositions are used in a critical care center. In other embodiments, compositions of the invention are used at home. In other embodiments, compositions of the invention are components of a kit or first-aid kit. In other embodiments, compositions of the invention are used in the field, with non-limiting examples comprising the use of compositions at the scene of an accident or on a battlefield. In other embodiments, compositions of the invention are used by either the military or civilians.

In some embodiments, compositions of the invention are administered via injection to a subject. In some embodiments, the subject is suffering from sepsis, burns, trauma, fluid loss, acid-base imbalance, electrolyte imbalance, or kidney injury. In some embodiments, compositions of the invention are used to administer a fluid to the subject. In some embodiments, compositions of the invention are used to hydrate or rehydrate a subject. In some embodiments, compositions are used to maintain adequate blood pressure in a subject. In some embodiments, compositions of the invention are used as a replacement fluid. In some embodiments, compositions of the invention are used as a component or adjunct component of continuous renal replacement therapy. In some embodiments, the subject's blood is dialyzed against compositions of the invention during continuous renal replacement therapy.

In some method embodiments, a therapeutically effective amount of a composition embodiment of the invention and a second composition is administered to a subject. In some method embodiments, the second composition is a pharmaceutical drug. In some embodiments, the second composition is a treatment for cancer, cardiovascular disease, kidney disease, end stage renal disease, an eye disease, cystic fibrosis, a degenerative disease, a blood disorder, diabetes, metabolic syndrome, obesity, a wound, a skin irritation, inflammation, an injury, a medical condition requiring dialysis treatment, or a combination thereof.

In some embodiments, compositions of the invention are combined with a pharmaceutical drug, drug ingredient, or medical device. In some embodiments, compositions of the invention are used to administer the drug to a subject. In some embodiments, compositions of the invention are used for the manufacturing of a drug, drug ingredient, or medical device. In some embodiments, the drug comprises one used to treat cancer, eye disease or injury, ear disease, kidney disease or injury, cardiovascular disease, gastrointestinal disorder or disease, or an endocrinopathy, such as diabetes, metabolic syndrome, or obesity, cystic fibrosis, sepsis, fluid loss, infection, neurodegenerative disease a degenerative disease, a blood disorder, diabetes, a wound, a skin irritation, inflammation, an injury, a medical condition requiring dialysis treatment, or a combination thereof. Non-limiting symptoms of these diseases, which may be treated by embodiments of the invention, comprise vascular disease; chronic kidney disease; end stage renal disease; glomerular disease; tubular disease; kidney injury; acute kidney injury; sepsis-induced kidney injury; drug-induced kidney injury; hypovolemia-induced kidney injury; ischemic kidney injury; fibrosis; vascular disease; hypertension; salt-sensitive hypertension; heart attack; heart failure; cardiac remodeling; cardiac fibrosis; myocardial fibrosis; atherosclerosis; stroke; arterial stiffening; vascular wall thickening; thickening of the peritoneal membrane; dyslipidemia; blood clot; anemia; an acid-base imbalance; hypercholemia; infection; sepsis; thrombosis; coronary artery disease; ischemic heart disease; peripheral artery disease; heartburn; indigestion; nausea; vomiting; peptic ulcers; abdominal pain; belching; bloating; flatulence; gallstones; constipation; diarrhea; hemorrhoids; rectal problems; vision loss; central vision loss; peripheral vision loss; blindness; blurry vision; cloudy vision; distorted vision; pink eye; eye infection; bulging eyes; bumps on the eye or eyelid; burning sensation in eyes; crusty eyelids; altered depth perception; discharge from eyes; double vision; dry eyes or dry eyelids; the sensation of a foreign body in the eye; gritty sensation in the eye; corneal lesion; light flashes; spots on eye lid; yellow eyes; fever; chills; decreased urination; tachycardia; tachypnea; hyperventilation; presence of bacteria or fungi in the bloodstream; unexplained weight loss; extreme fatigue; pain; skin changes such as change in the color or size or shape of skin mole; chronic sores; one or more lumps in a tissue such as a lump in a breast or lymph node; bleeding or discharge; chronic cough; hearing loss; tinnitus; congestion; infection; type 1 diabetes; type 2 diabetes; type 3 diabetes; gestational diabetes; juvenile diabetes; latent autoimmune diabetes of adulthood; maturity onset diabetes of the young; insulin resistance; hyperglycemia; steroid-induced diabetes; brittle diabetes; diabetes insipidus; diabetes mellitus; hemoglobin A1c levels between above 5.7% and 6.4%; hemoglobin A1c levels above 6.4%; syndrome x; an individual displaying at least three of the following metabolic risk factors: waist size greater than 40 inches if male subject or greater than 35 inches if female subject, blood tryglyceride levels of at least about 150 mg/dl or higher or currently using a cholesterol medication, blood high-density lipoprotein levels lower than about 40 mg/dl if male subject or lower than about 50 mg/dl if female subject or currently using a cholesterol medication, blood pressure above about 135/85 mm Hg (systolic over diastolic) or using a high blood pressure medication, and fasting blood glucose levels of about 100 mg/dl or higher; or a combination thereof. In some embodiments, the drug or device is used after a surgical operation. In some embodiments, the drug or device is used to promote tissue regeneration. In some embodiments, the drug is chemically defined as a protein, recombinant protein, cellular therapeutic, antibody, biologic, molecule, liposome, lipid, deoxyribonucleic acid, ribonucleic acid, or small molecule. In some embodiments, the drug is a blood pressure controlling agent, an angiotensin-converting enzyme inhibitor, an angiotensin receptor blocker, a beta blocking agent, an alpha blocking agent, an anti-arrhythmic agent, a blood thinner, an alpha agonist, a sodium channel blocking agent, a calcium channel blocking agent, an anti-platelet agent, an anti-hyperlipidemic agent, a statin, a nonsteroidal anti-inflammatory drug, a loop diuretic, a thiazide diuretic, a potassium-sparing diuretic, a vasodilator, a renin inhibitor, dopamine, a dopamine receptor agonist, a thrombolytic agent, erythropoietin, an erythropoietic stimulating agent, a vitamin, a vitamin analogue, a drug used in the management of ESRD, an anti-infective agents, an antibiotic, an antifungal, a cancer chemotherapeutic agent, a steroid, an injectable drug, a topical drug, an eye drop or ointment, a drying agent for the ear, an anti-inflammatory drug, a prostaglandin analogue, a carbonic anhydrase inhibitor, an adenergic agonist, an anti-allergy drug, an angiogenesis inhibitor drug, an 5-aminosalicylates, an antacid, an anti-diarrheal, a digestive enzyme, a chloride channel activator, a guanylate cyclase-C agonist, a peripheral opioid receptor agonist, a peripheral opioid receptor antagonist, a gallstone solubilizing agent, a gastrointestinal stimulant, a Helicobacter pylori eradication agent, a histamine-2(H2) blocker, an antiacid, a laxative, an osmotic laxative, a polyethylene glycol osmotic, a gastrointestinal simulant, a promotility agent, a stool softener, a bulk-forming fiber, a proton pump inhibitor, a flu shot, an infectious-disease vaccine, a cancer vaccine, an insulin agent, an amylinomimetic agent, an alpha-glucosidase inhibitor, a biguanide, a dopamine agonist, a glucagon-like peptide, a meglitinide agent, a sodium glucose transporter 2 inhibitor, a sulfonylurea agent, a thiazolidinedione agent, and a dipeptidyl peptidase-4 inhibitor, or a combination thereof. Non-limiting examples of drugs include captopril, enalapril, fosinopil, lisinopril, perindopril, quinapril, trandolapril, benazepril, ramipril, azilsartan, candesartan, telmisartan, fimasartan eprosartan, irbesartan, losartan, olmesartan, valsartan, doxazosin, phentolamine, indoramin, phenoxybenzamine, tolazoline, bucindolol, carvedilol, labetalol, tamsulosin, terazosin, prazosin, alfuzosin, timolol, betaxolol, propranolol, atenolol, nadolol, nebivolol, oxprenolol, pindolol, propranolol, metoprolol, sodium nitroprusside, hydralazine, adenosine, sildenafil, vardenafil, tadalafil, prostacyclin, nitric oxide, amiodarone, mexiletine, disopryamide, propafenone, diltiazem, dihydropyridines, amlodipine, cilnidipine, felodipine, isradipine, nimodipine, lercanidipine, levamlodipine, nicardipine, nitrendipine, nifedipine, verapamil, spironolactone, bumetanide, ethacrynic acid, epitizide, metolazone, amiloride, triamterene, torsemide, furosemide, indapamide, triamterene, hydrochlorothiazide, chlorothiazide, bendroflumethiazide, chlorthalidone, celecoxib, meloxicam, ibuprofen, naproxen, diclofenac, aspirin, dipyridamole, clopidogrel, cilostazol, ticlopidine, lovastatin, niacin, simvastatin, ezetimibe, warfarin, carperitide (recombinant ANP), nesiritide (recombinant BNP), tinzaparin, enoxaparin, heparin, atorvastatin, fluvastatin, pravastatin, rosuvastatin, aliskiren, alteplase, anistreplase, reteplase, tenecteplase, streptokinase, tissue plasminogen activator, urokinase, recombinant human erythropoietin, epoetin alpha, epoetin beta, darbepoetin alpha, methoxy polyethylene glycol-epoetin beta, Epo, Procrit®, Epogen®, Aranesp, Mircera, vitamin D, rocaltrol, calcitriol, zemplar®, hectorol, doxercalciferol, carnitor, levocarntine, lepiridun, reteplase, alteplase, peginesatide, iron, sodium ferric gluconate, vitamin B12, Darbepoetin, midazolam hydrochloride, diazepam, calcium gluconate, calcitonin, deferoxamine, doxercalciferol, ibandronate, pamidronate, paricalcitol, methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, penicillin, amoxicillin, cephalexin, erythromycin, clarithromycin, azithromycin, ciprofloxacin, levofloxacin, ofloxacin, sulfamethoxazole, trimethoprim, fosfomycin, nitrofurantoin, ceftriaxone, clavulanate, clindamycin, doxycycline, tetracycline, clotrimazole, econazole nitrate, miconazole, terbinafine, fluconazole, ketoconazole, and amphotericin. Non-limiting examples of chemotherapy and radiotherapy that may be used with embodiments of the invention comprise methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, x-rays, gamma rays, and charged particles. In some embodiments, the drug comprises regular insulin such as but not limited to Humulin or Novolin, insulin aspart such as but not limited to Novolog or FlexPen; insulin glulisine such as but not limited to Apidra; insulin lispro such as but not limited to Humalog; insulin isophane such as but not limited to Humulin N or Novolin N; insulin degludec such as but not limited to Tresiba; insulin detemir such as but not limited to Levemir; insulin glargine such as but not limited to Lantus; insulin glargine such as but not limited to Toujeo; a combination insulin drug such as but not limited to insulin aspart protamine-insulin aspart, insulin lispro protamine-insulin lispro, human isophane insulin-human insulin regular, insulin dedludec-insulin aspart, NovoLog Mix 70/30, Humalog Mix 75/25, Humalog Mix 50/50, Humalin 70/30, Novolin 70/30, or Ryzodeg; pramlintide such as but not limited to SymlinPen; acarbose such as but not limited to Precose; miglitol such as but not limited to Glyset; metformin such as but not limited to Glucophage, Metformin Hydrochloride ER, Glumetza, Riomet, or Fortamet; a metformin-containing drug such as but not limited to metformin-alogliptin, Kazano, metformin-canagliflozin, Invokamet, metformin-dapagliflozin, Xigduo XR, metformin-empagliflozin, Synjardy, metformin-glipizide, metformin-glyburide, Glucovance, metformin-linagliptin, Jentadueto, metformin-pioglitazone, Actoplus, Actoplus Met, Actoplus Met XR, metformin-repaglinide, PrandiMet, metformin-rosiglitazone, Avandamet, metformin-saxagliptin, Kombiglyze XR, metformin-sitagliptin, Janumet, or Janumet XR; bromocriptine such as but not limited to Parlodel; alogliptin such as but not limited to Nesina; alogliptin-pioglitazone such as but not limited to Oseni; linagliptin such as but not limited to Tradjenta, linagliptin-empagliflozin such as but not limited to Glyzami; saxagliptin such as but not limited to Onglyza; sitagliptin such as but not limited to Januvia; sitagliptin and simvastatin such as but not limited to Juvisync; albiglutide such as but not limited to Tanzeum; dulaglutide such as but not limited to Trulicity; exenatide such as but not limited to Byetta; exenatide extended-release such as but not limited to Bydureon; liraglutide such as but not limited to Victoza; nateglinide such as but not limited to Starlix; repaglinide such as but not limited to Prandin; dapagliflozin such as but not limited to Farxiga; canaglifoxin such as but not limited to Invokana; empaglifozin such as but not limited to Jardiance; empagliflozin-linagliptin such as but not limited to Glyxambi; glimepiride such as but not limited to Amaryl; glimepiride-pioglitazone such as but not limited to Duetact; glimepiride-rosiglitazone such as but not limited to Avandaryl; gliclazide, glipizide such as but not limited to Glucotrol; glyburide such as but not limited to DiaBeta, Glynase, or Micronase; chlorpropamide such as but not limited to Diabinese; tolazamide such as but not limited to Tolinase; tolbutamide such as but not limited to Orinase or TolTab; rosiglitazone such as but not limited to Avandia; pioglitazone such as but not limited to Actos, or a combination thereof. In some embodiments, the treatment regimen includes administration of one or more pharmaceutical drugs, each administered separately to a subject; behavioral modification such as dietary changes and increased daily exercise; or surgery such as bariatric surgery.

In some embodiments, compositions of the invention are administered to a subject with cancer. In some embodiments, the cancer comprises solid tumor; metastatic tumor; epithelial cancer; circulating cancer cells; eye cancer; kidney cancer; childhood cancer; brain cancer; spinal cord tumor; liver cancer; bone cancer; colorectal cancer; stomach cancer; small intestine cancer; prostate cancer; breast cancer; skin cancer; basal cell cancer; squamous cell skin cancer; melanoma; multiple myeloma; lung cancer; lung tumor; small cell lung cancer; non-small cell lung cancer; blood cancer; leukemia; lymphoma; Hodgkin's lymphoma; non-Hodgkin's lymphoma; bladder cancer; oral cancer; oropharyngeal cancer; pancreatic cancer;

thyroid cancer; thymus cancer; uterine cancer; uterine sarcoma; cervical cancer; ovarian cancer; testicular cancer; Wilms tumor; acute lymphocytic leukemia, chronic lymphocyte leukemia; acute myeloid leukemia; chronic myeloid leukemia; chronic myelomonocytic leukemia; adrenal cancer; anal cancer; bile duct cancer; endometrial cancer; esophagus cancer; a Ewing tumor; gallbladder cancer; gastrointestinal tumor; Kaposi sarcoma; laryngeal cancer; hypopharyngeal cancer; malignant mesothelioma; Merkel cell skin cancer; myelodysplastic syndrome; cancer of the nasal cavity; paranasal sinus cancer; nasopharyngeal cancer; neuroblastoma; osteosarcoma; penile cancer; pituitary tumor; retinoblastoma; rhabdomyosarcoma; salivary gland cancer; soft tissue sarcoma; vaginal cancer; vulvar cancer, or a combination thereof. In some embodiments, the cancerous tissue is found in the bone, bone marrow, blood, lymph, lymph nodes, immune cells, breast, skin, prostate, ovary, cervix, uterus, vagina, penis, testicles, bladder, intestine, colorectal region, pancreas, bile duct, stomach, gastrointestinal tract, thyroid, soft tissue, lung, liver, kidney, adrenal glands, brain, spinal region, central nervous system, peripheral nervous system, eye, gums, lips, mouth, salivary gland, tonsils, jaw, esophagus, throat, sinus, nasopharyngeal region, head and neck region, heart, or a combination thereof. In some embodiments, compositions of the invention are administered to a subject with cancer through a medical tube, such as but not limited to a central line. In some embodiments, compositions of the invention are used to flush a medical line or preserve an injection site in a subject receiving cancer treatments. In some embodiments, compositions of the invention are combined with a cancer drug, such as but not limited to a cancer chemotherapeutic drugs, and administered to a subject.

In some embodiments, compositions of the invention are administered to a subject with eye disease. In some embodiments, the eye disease comprises a retinal disease, a neurodegenerative eye disease, macular degeneration, glaucoma, dry eye disease, keratoconus, or a combination thereof. In some embodiments, compositions of the invention are administered by injection or by topical administration. In some embodiments, compositions of the invention are administered to a subject using a needle, syringe, tube, dropper, bottle, contact lens, or combination thereof. In some embodiments, compositions of the invention are administered to a subject who before, during, or after a medical operation is performed in one or both of the subject's eyes, orbital sockets, or a combination thereof. For example, compositions of the invention may be administered to a subject who receives laser assisted in situ keratomileusis (LASIK). As another example, compositions of the invention may be administered to a subject who receives cataract surgery.

Some embodiments of the invention comprise a method of treating a subject comprising administering an effective amount of a composition as described herein, such as a composition comprising a concentration of bromide ions (Br⁻) between 30 μM and 1 mM and a concentration of chloride ions (Cl⁻) between 17.1 and 200 mM. In some embodiments, the composition is used in place of a Br-deficient saline. In some embodiments, the composition is used in a critical care center, in a hospital, or as a field dressing. In some embodiments, the composition is administered via injection, topical, or oral route of administration. In some embodiments, the composition is administered using routes of administration as described herein. In some method embodiments, the topical administration involves irrigation, debridement, washing, hydrating, perfusing, soaking, or perfusion.

Some embodiments of the invention comprise methods of treating or preventing a Br⁻ ion dependent disease in a subject, the method comprising administering a therapeutically effective amount of a composition comprising an amount of Br⁻ and Cl⁻ ions wherein molar amount of Br⁻ ions is between about 0.0338% and about 4.613% of the molar amount of Cl⁻. In some method embodiments, the Br⁻ ion dependent disease comprises cancer, cardiovascular disease, kidney disease, end stage renal disease, an eye disease, cystic fibrosis, a degenerative disease, a blood disorder, diabetes, a wound, a skin irritation, inflammation, an injury, a medical condition requiring dialysis treatment, or a combination of the diseases listed herein.

In embodiments, compositions of the invention are administered to an animal, such as but not limited to a cow, horse, sheep, goat, pig, bird, chicken, turkey, dog, cat, mouse, rabbit, rat, hamster, or guinea pig. Some embodiments of the invention can be administered to an animal to treatment or preventative treatment for heart disease. For example, embodiments of the invention can be administered to pigs for the prevention of Mulberry heart disease, to cows for the prevention or treatment of brisket disease, to poultry for the prevention of ascites, or to combinations thereof. Some embodiments can be administered to a turkey to prevent hypertensive angiopathy, sudden death syndrome, or perirenal hemorrhage syndrome. Some embodiments of the invention can be administered to an animal as a treatment or preventative treatment for kidney disease, such as in a dog or cat. Some embodiments can be administered to improve the survival of a dog, cat, mouse, rabbit, rat, hamster, guinea pig, turkey, chicken, cow, pig, goat, sheep, or horse. Some embodiments can be administered to a flock, herd, or population of animals to improve production yields within the flock, herd, or population. Some embodiments can be administered as a food or drink. Some embodiments can be administered as a drug, a vaccine, an inoculation, a shot, an injectable drug, a pill, a powder, or a patch. Some embodiments can be administered in a field, roofed enclosure, pen, or feedlot. Some embodiments can be administered to an animal, flock, or herd by a veterinarian. Other embodiments can be administered to an animal, flock, or herd by a farmer or rancher. Embodiments can be manufactured, packaged, and distributed as a pre-mixed food or food supplement for animals.

In some embodiments, compositions of the invention are manufactured by adding an effective amount of Br⁻ to a saline solution, such as a Br-deficient saline solution. A Br-deficient saline solution, for example, comprises an amount less than about 30 μM bromide ions. In embodiments, an amount of Br⁻ can be added to a Br-deficient saline solution to manufacture a solution with a physiologically adequate amount of Br⁻. For example, a physiologically adequate amount of Br⁻ is an amount between about 30 μM or 1 mM bromide ions. For example, embodiments of the invention can be manufactured from a pre-made, Br-deficient saline solution, with non-limiting examples comprising 0.9% NaCl, Ringer's solution, and Hartmann's solution. Some embodiments of the invention are manufactured by adding Br⁻ to a Br-deficient saline solution, such as 0.9% NaCl, where only an amount of Br⁻ needed to bring the final concentration of Br⁻ to an amount between about 30 μM and about 1 mM Br⁻.

Embodiments of the invention can provide physiologically balanced levels of bromide ions and chloride ions to a subject.

Some embodiments of the invention comprise physiologically effective amounts of Br⁻, Cl⁻, and Mg²⁺. Some embodiments of the invention comprise a dialysate comprising an amount between about 30 μM and about 1 mM Br⁻, an amount between about 80 mM and about 200 mM Cl⁻, and an amount between about 0.75 mM and about 3 mM Mg²⁺. In some embodiments, the amount of Br⁻ is about 30 μM, 40 μM, 45 μM, 50 μM, 55 μM, 60 μM, 65 μM, 75 μM, 80 μM, 85 μM, 90 μM, 95 μM, 100 μM, 105 82 M, 110 μM, 115 μM, 120 μM, 125 μM, 130 μM, 135 μM, 140 μM, 145 μM, 150 μM, 155 μM, 160 μM, 170 μM, 175 μM, 180 μM, 185 μM, 190 μM, 195 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1 mM, or any other amount between about 30 μM and about 1 mM. In some embodiments, the amount of Cl⁻ is about 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, or any other amount between about 80 mM and about 200 mM. In some embodiments, the amount of Mg²⁺ is about 0.75 mM, 0.80 mM, 0.85 mM, 0.90 mM, 0.95 mM, 1.05 mM, 1.05 mM, 1.10 mM, 1.15 mM, 1.20 mM, 1.25 mM, 1.30 mM, 1.35 mM, 1.40 mM, 1.45 mM, 1.50 mM, 1.55 mM, 1.60 mM, 1.65 mM, 1.70 mM, 1.75 mM, 1.80 mM, 1.85 mM, 1.90 mM, 1.95 mM, 2.00 mM, 2.05 mM, 2.10 mM, 2.15 mM, 2.20 mM, 2.25 mM, 2.30 mM, 2.35 mM, 2.40 mM, 2.45 mM, 2.50 mM, 2.55 mM, 2.60 mM, 2.65 mM, 2.70 mM, 2.75 mM, 2.80 mM, 2.85 mM, 2.90 mM, 2.95 mM, 3.0 mM, or any other amount between about 0.75 mM and about 3.0 mM.

Some compositions of the invention described herein comprise concentrated dialysate fluids that upon dilution yields a final dialysate comprising an amount between about 30 μM and about 1 mM Br⁻, an amount between about 80 mM and about 200 mM Cl⁻, and an amount between about 0.75 mM and about 3 mM Mg²⁺. In some embodiments, the compositions can comprise a dry powder that upon reconstitution with a biocompatible fluid yields a final dialysate comprising an amount between about 30 μM and about 1 mM Br⁻, an amount between about 80 mM and about 200 mM Cl⁻, and an amount between about 0.75 mM and about 3 mM Mg²⁺. In some embodiments, compositions of the invention described herein can comprise the contemplated composition that is mixed with a second composition to yield a final dialysate comprising an amount between about 30 μM and about 1 mM Br⁻, an amount between about 80 mM and about 200 mM Cl⁻, and an amount between about 0.75 mM and about 3 mM Mg²⁺, such as but not limited to an acidified embodiment of the compositon that is combined with a basic composition to yield a final dialysate comprising an amount between about 30 μM and about 1 mM Br⁻, an amount between about 80 mM and about 200 mM Cl⁻, and an amount between about 0.75 mM and about 3 mM Mg²⁺. As another non-limiting embodiment, compositions of the invention described herein can comprise a basic (eg high pH) composition embodiment that is combined with an acidic composition to yield a final dialysate comprising an amount between about 30 μM and about 1 mM Br⁻, an amount between about 80 mM and about 200 mM Cl⁻, and an amount between about 0.75 mM and about 3 mM Mg²⁺. Some embodiments comprise only those amounts of Br⁻ required to maintain a subject's serum Br⁻ between about 30 μM and about 1 mM. Some embodiments comprise only those amounts of Mg²⁺ required to maintain a subject's serum Mg²⁻ between about 2.5 mg/dl and about 5.0 mg/dl.

Some composition embodiments of the invention are formulated as a dialysate and used to perform dialysis on a subject. In some embodiments, the dialysate also comprises an amount of bicarbonate, potassium, sodium, calcium, glucose, dextrose, acetate, citric acid, or any combination thereof. Some embodiments, the compositions comprise a physiologically balanced amount of Br⁻, Cl⁻, Mg²⁺, and one or more of an amount of bicarbonate, potassium, sodium, calcium, glucose, dextrose, acetate, citric acid, or any combination thereof. Some embodiments of the invention comprise a dialysate or replacement fluid. An example of a dialysate comprises a solution formulated for use during acute dialysis and maintenance dialysis. An example of a replacement fluid comprises a solution formulated for use during acute dialysis.

Some embodiments of the invention comprise methods of preventing the development of a morbidity or mortality event in a subject. Some embodiments comprise performing dialysis on the subject using compositions as described herein. For example, embodiments comprise performing dialysis on a subject using a dialysate or replacement fluid that contains an amount between about 30 μM and about 1 mM Br⁻, an amount between about 80 mM and about 200 mM Cl⁻, and an amount between about 0.75 mM and about 3 mM Mg²⁺. In some embodiments, the subject can be deficient in Br⁻, Mg²⁺, or a combination thereof. Some embodiments can correct or prevent Mg-deficiency in a subject. Some embodiments can correct or prevent Mg-deficiency as well as Br-deficiency in a subject. In some embodiments, Mg²⁺ levels can be measured in the blood, serum, plasma, urine, or used dialysate of a subject. In some embodiments, Br⁻ levels can be measured in the blood, serum, plasma, urine, or used dialysate of a subject.

Some embodiments of the invention comprise a method of preventing cardiovascular disease or mortality in a subject by administering a composition as described here. In some embodiments, the subject is being treated with dialysis. Some embodiments of the invention comprise a method of preventing cardiovascular disease or mortality in a subject, the method comprising performing dialysis on the subject against a dialysis fluid comprising a sufficient amount of Br⁻ to maintain the subject's serum Br⁻ between about 30 μM and about 1 mM and a sufficient amount of Mg²⁺ to maintain the subject's serum Mg²⁺ between about 2.5 mg/dl and about 3.0 mg/dl.

In some embodiments, compositions as described herein further comprise one of more biologically active agents. A non-limiting example of a biologically active agent comprises a pharmaceutical drug. In some embodiments, compositions as described herein are administered before, at about the same time as, or after the administration of a separate composition, such as a biologically active agent. In some embodiments, a composition as described herein can be combined with a drug and be used in the treatment or prevention of a disease, such as cancer, cardiovascular disease, kidney disease, end stage renal disease, an eye disease, cystic fibrosis, a degenerative disease, a blood disorder, diabetes, a wound, a skin irritation, inflammation, an injury, or a medical condition requiring dialysis treatment. Non-limiting examples of the drug comprise a blood pressure controlling agent, an angiotensin-converting enzyme inhibitor, an angiotensin receptor blocker, a beta blocking agent, an alpha blocking agent, an anti-arrhythmic agent, a blood thinner, an alpha agonist, a sodium channel blocking agent, a calcium channel blocking agent, an anti-platelet agent, an anti-hyperlipidemic agent, a statin, a nonsteroidal anti-inflammatory drug, a loop diuretic, a thiazide diuretic, a potassium-sparing diuretic, a vasodilator, a renin inhibitor, dopamine, a dopamine receptor agonist, a thrombolytic agent, erythropoietin, an erythropoietic stimulating agent, a vitamin, a vitamin analogue, a drug used in the management of ESRD, an anti-infective agents, an antibiotic, an antifungal, a cancer chemotherapeutic agent, a steroid, an injectable drug, a topical drug, an eye drop or ointment, a drying agent for the ear, an anti-inflammatory drug, a prostaglandin analogue, a carbonic anhydrase inhibitor, an adenergic agonist, an anti-allergy drug, an angiogenesis inhibitor drug, an 5-aminosalicylates, an antacid, an anti-diarrheal, a digestive enzyme, a chloride channel activator, a guanylate cyclase-C agonist, a peripheral opioid receptor agonist, a peripheral opioid receptor antagonist, a gallstone solubilizing agent, a gastrointestinal stimulant, a Helicobacter pylori eradication agent, a histamine-2(H2) blocker, an antacid, a laxative, an osmotic laxative, a polyethylene glycol osmotic, a gastrointestinal simulant, a promotility agent, a stool softener, a bulk-forming fiber, a proton pump inhibitor, a flu shot (e.g., a vaccine), an infectious-disease vaccine, a cancer vaccine, a drug used in the management of diabetes or metabolic syndrome, or a combination thereof.

In some method embodiments, the composition is contacted with a medical device. In some method embodiments, the medical device comprises a surgical device, a syringe, a needle, a suture, a contact lens, a medical device used in dialysis, a dialysis machine, a port, a fistula, a catheter, a graft, a machine used in the medical care of a subject, a component of a machine used in the medical care of a subject, a stent, a plug, a patch, an injection filler, a powder, a bandage, a gauze, a negative pressure device, a hydrophobic dressing, a hemostatic agent, an absorbent, a hydrating agent, an osmotic agent, a preservative, a wash, a lotion, a cream, a gel, a paste, an ointment, a rinse, an eye drop, an ear drop, a spray, a nasal spray, a matrix, a scaffold, an acellular scaffold, a collagen-based device, a camera, a pill camera, a tube, a connector, a needle-free connector, a closed system, transfer device, a hemodynamic monitoring system, a bag, a balloon, an intravenous line, a central line, an arterial line, or a combination thereof. In some embodiments, the composition is used to cleanse, wash, moisten, hydrate, perfuse, or lubricate the medical device.

In some embodiments, compositions of the invention are contacted with an organ, a tissue, or a tissue product. Non-limiting examples of organs, tissues, or tissue products comprise harvested tissues, engineered tissues, cultured tissues, human tissues, animal tissues, autologous tissues, bone, ligaments, tendons, vascularized organs, lung, kidney, heart, heart tissue, liver, pancreas, corneas, blood, stem cells, progenitor cells, skin, dura mater, oocytes, semen, and combinations thereof. In some embodiments, the composition is used to cleanse, wash, moisten, hydrate, perfuse, or lubricate the organ, tissue, or tissue product.

In some embodiments, the subject has a medical condition, non-limiting examples of which comprise cancer, eye disease or injury, ear disease, kidney disease or injury, cardiovascular disease, gastrointestinal disorder or disease, or an endocrinopathy, (such as diabetes, metabolic syndrome, or obesity), cystic fibrosis, sepsis, fluid loss, infection, neurodegenerative disease, a degenerative disease, a blood disorder, diabetes, a wound, a skin irritation, inflammation, an injury, or a medical condition requiring dialysis treatment. In some embodiments, the medical condition is caused by a medical operation, disease, infection, trauma, or natural aging process. In some embodiments, the medical condition comprises a chronic wound, dermal ulcer, pressure ulcer, mouth sore, diabetic ulcer, sepsis, fluid low, acid-base imbalance, electrolyte imbalance, blood cancer, solid tumor, recurrent cancer, or metastatic tumor.

Embodiments of the invention provide compositions and methods for promoting the health of the heart, kidneys, and/or vasculature in a subject. Compositions and methods that promote the health of these organs before they deteriorate into disease states may reduce the burden of cardiovascular disease and/or kidney disease. Embodiments of the invention are unified by the common feature of utilizing a nutritionally effective amount of bromide salt (Br) to promote heart, kidney, and/or vasculature health in a subject. In some embodiments, the subject is a human. In other embodiments, the subject is a non-human animal, such as but not limited to a dog, cat, mouse, rabbit, rat, hamster, guinea pig, horse, cow, goat, sheep, pig, chicken, or turkey.

Embodiments of the invention comprise a nutritional composition for promoting heart health, kidney health, vascular health or a combination thereof in a human subject, the composition comprising between about 0.1 mg and about 50 mg Br⁻. In embodiments, the nutritional composition comprises about 0.1 mg, 0.5 0 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 20.5 mg, 21.0 mg, 21.5 mg, 22.0 mg, 22.5 mg, 23.0 mg, 23.5 mg, 24.0 mg, 24.5 mg, 25.0 mg, 25.5 mg, 26.0 mg, 26.5 mg, 27.0 mg, 27.5 mg, 28.0 mg, 28.5 mg, 29.0 mg, 29.5 mg, 30.0 mg, 30.5 mg, 31.0 mg, 31.5 mg, 32.0 mg, 32.5 mg, 33.0 mg, 33.5 mg, 34.0 mg, 34.5 mg, 35.0 mg, 35.5 mg, 36.0 mg, 36.5 mg, 37.0 mg, 37.5 mg, 38.0 mg, 38.5 mg, 39.0 mg, 39.5 mg, 40.0 mg, 40.5 mg, 41.0 mg, 41.5 mg, 42.0 mg, 42.5 mg, 43.0 mg, 43.5 mg, 44.0 mg, 44.5 mg, 45.0 mg, 45.5 mg, 46.0 mg, 46.5 mg, 47.0 mg, 47.5 mg, 48.0 mg, 48.5 mg, 49.0 mg, 49.0 mg, 49.5 mg, 50.0 mg, or any other amount between about 0.1 mg and about 50 mg of Br⁻.

Some embodiments of the invention comprise a composition that is administered to an animal subject from about 0.0001 mg Br⁻ per kg body weight of the subject to about 0.1 mg Br⁻ per kg body weight of the subject. Non-limiting examples of animal subjects include dog, cat, mouse, rabbit, rat, hamster, guinea pig, mouse, rabbit, rat, hamster, guinea pig, horse, cow, goat, sheep, pig, chicken, or turkey. Some embodiments, the compositions described herein is administered to a subject at about 0.0001 mg, 0.0005 mg, 0.0010 mg, 0.0015 mg, 0.0020 mg, 0.0025 mg, 0.0030 mg, 0.0035 mg, 0.0040 mg, 0.0045 mg, 0.0050 mg, 0.0055 mg, 0.0060 mg, 0.0065 mg, 0.0070 mg, 0.0075 mg, 0.0080 mg, 0.0085 mg, 0.0090 mg, 0.0095 mg, 0.0100 mg, 0.0105 mg, 0.0110 mg, 0.0115 mg, 0.0120 mg, 0.0125 mg, 0.0130 mg, 0.0135 mg, 0.0140 mg, 0.0145 mg, 0.0150 mg, 0.0155 mg, 0.0160 mg, 0.0165 mg, 0.0170 mg, 0.0175 mg, 0.0180 mg, 0.0185 mg, 0.0190 mg, 0.0195 mg, 0.0200 mg, 0.0205 mg, 0.0210 mg, 0.0215 mg, 0.0220 mg, 0.0225 mg, 0.0230 mg, 0.0235 mg, 0.0240 mg, 0.0245 mg, 0.0250 mg, 0.0255 mg, 0.0260 mg, 0.0265 mg, 0.0270 mg, 0.0275 mg, 0.0280 mg, 0.0285 mg, 0.0290 mg, 0.0295 mg, 0.0300 mg, 0.0305 mg, 0.0310 mg, 0.0315 mg, 0.0320 mg, 0.0325 mg, 0.0330 mg, 0.0335 mg, 0.0340 mg, 0.0345 mg, 0.0350 mg, 0.0355 mg, 0.0360 mg, 0.0365 mg, 0.0370 mg, 0.0375 mg, 0.0380 mg, 0.0385 mg, 0.0390 mg, 0.0395 mg, 0.0400 mg, 0.0405 mg, 0.0410 mg, 0.0415 mg, 0.0420 mg, 0.425 mg, 0.0430 mg, 0.0435 mg, 0.0440 mg, 0.0445 mg, 0.0450 mg, 0.0455 mg, 0.0460 mg, 0.0465 mg, 0.0470 mg, 0.0475 mg, 0.0480 mg, 0.0485 mg, 0.0490 mg, 0.0495 mg, 0.0500 mg, 0.0505 mg, 0.0510 mg, 0.0515 mg, 0.0520 mg, 0.0525 mg, 0.0530 mg, 0.0535 mg, 0.0540 mg, 0.0545 mg, 0.0550 mg, 0.0555 mg, 0.0560 mg, 0.0565 mg, 0.0570 mg, 0.0575 mg, 0.0580 mg, 0.0585 mg, 0.0590 mg, 0.0595 mg, 0.0600 mg, 0.0605 mg, 0.0610 mg, 0.0615 mg, 0.0620 mg, 0.0625 mg, 0.0630 mg, 0.0635 mg, 0.0640 mg, 0.0645 mg, 0.0650 mg, 0.0655 mg, 0.0660 mg, 0.0665 mg, 0.0670 mg, 0.0675 mg, 0.0680 mg, 0.0685 mg, 0.0690 mg, 0.0695 mg, 0.0700 mg, 0.0705 mg, 0.0710 mg, 0.0715 mg, 0.0720 mg, 0.0725 mg, 0.0730 mg, 0.0735 mg, 0.0740 mg, 0.0745 mg, 0.0750 mg, 0.0755 mg, 0.0760 mg, 0.0765 mg, 0.0770 mg, 0.0775 mg, 0.0780 mg, 0.0785 mg, 0.0790 mg, 0.0795 mg, 0.0800 mg, 0.0805 mg, 0.0810 mg, 0.0815 mg, 0.0820 mg, 0.0825 mg, 0.0830 mg, 0.0835 mg, 0.0840 mg, 0.0845 mg, 0.0850 mg, 0.0855 mg, 0.0860 mg, 0.0865 mg, 0.0870 mg, 0.0875 mg, 0.0880 mg, 0.0885 mg, 0.0890 mg, 0.0895 mg, 0.0900 mg, 0.0905 mg, 0.0910 mg, 0.0915 mg, 0.0920 mg, 0.0925 mg, 0.0930 mg, 0.0935 mg, 0.0940 mg, 0.0945 mg, 0.0950 mg, 0.0955 mg, 0.0960 mg, 0.0965 mg, 0.0970 mg, 0.0975 mg, 0.0980 mg, 0.0985 mg, 0.0990 mg, 0.095 mg, 0.1000 mg, or any other amount between about 0.0001 mg and about 0.1000 Br⁻ per kg body weight of the subject.

In certain embodiments, the compositions of the invention can be administered as a nutritionally effective amount of Br⁻ to a subject. As used herein, a nutritionally effective amount of Br⁻ refers to the amount of Br⁻ or Br⁻ composition needed to maintain the circulating levels of Br⁻ to those concentrations that are required for eliciting the desired biological response following administration. For example, the desired biological response can be preventing cardiovascular disease in a subject and a nutritionally effective amount of Br⁻ can be the amount of Br⁻ needed to maintain the serum Br⁻ levels in the subject between 50 μM and 1 mM. In embodiments, a nutritionally effective amount is between about 0.1 mg of Br⁻ and 50 mg of Br⁻. In embodiments, a nutritionally effective amount of Br— is about 0.1 mg, 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 20.5 mg, 21.0 mg, 21.5 mg, 22.0 mg, 22.5 mg, 23.0 mg, 23.5 mg, 24.0 mg, 24.5 mg, 25.0 mg, 25.5 mg, 26.0 mg, 26.5 mg, 27.0 mg, 27.5 mg, 28.0 mg, 28.5 mg, 29.0 mg, 29.5 mg, 30.0 mg, 30.5 mg, 31.0 mg, 31.5 mg, 32.0 mg, 32.5 mg, 33.0 mg, 33.5 mg, 34.0 mg, 34.5 mg, 35.0 mg, 35.5 mg, 36.0 mg, 36.5 mg, 37.0 mg, 37.5 mg, 38.0 mg, 38.5 mg, 39.0 mg, 39.5 mg, 40.0 mg, 40.5 mg, 41.0 mg, 41.5 mg, 42.0 mg, 42.5 mg, 43.0 mg, 43.5 mg, 44.0 mg, 44.5 mg, 45.0 mg, 45.5 mg, 46.0 mg, 46.5 mg, 47.0 mg, 47.5 mg, 48.0 mg, 48.5 mg, 49.0 mg, 49.0 mg, 49.5 mg, 50.0 mg, or any other amount between about 0.1 mg and about 50.0 mg of Br⁻. For example, a nutritionally effective amount of Br⁻ is between about 1 and about 50 mg of Br⁻ that is administered to a human subject within a single 24 hour period. The Br⁻ may be administered as a single dose or as multiple smaller doses that collectively administer a nutritionally effective amount of Br⁻ to the human or animal subject. In some embodiments, the invention is used to promote the health of the heart, kidneys, and/or vasculature in a subject.

Some nutritional embodiments of the invention also comprise magnesium, vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid), vitamin B₁₂ (cobalamin), vitamin C (ascorbic acid), vitamin E (tocopherol), selenium, zinc, iron, vitamin D, or a combination thereof. In some embodiments, the nutritional composition comprises one or more of the following:
50-600 mg magnesium,
1.2-1.5 mg of vitamin B₁ (thiamine),
1.3-1.7 mg of vitamin B₂ (riboflavin),
16-20 mg of vitamin B₃ (niacin),
10 mg of vitamin B₅ (pantothenic acid),
1.7-100 mg of vitamin B₆ (pyridoxine),
30-300 μg of vitamin B₇ (biotin),
0.4-5 mg of vitamin B₉ (folic acid),
2.4 μg-2 mg of vitamin B₁₂ (cobalamin),
60-100 mg of vitamin C (ascorbic acid),
30 I.U. of vitamin E (tocopherol),
70 μg of selenium,
11-25 mg of zinc,
18-29 mg of iron, and/or
400-2000 I.U. of vitamin D.

In some embodiments, compositions can comprise at least two or more of the following:
About 5 mg Br⁻ (NaBr),
About 100 mg magnesium (MgCl₂),
About 1.2 mg of vitamin B₁ (thiamine),
About 1.7 mg of vitamin B₂ (riboflavin),
About 20 mg of vitamin B₃ (niacin),
About 10 mg of vitamin B₅ (pantothenic acid),
About 50 mg of vitamin B₆ (pyridoxine),
About 300 μg of vitamin B₇ (biotin),
About 0.8 mg of vitamin B₉ (folic acid),
About 6 μg of vitamin B₁₂ (cobalamin), and
About 100 mg of vitamin C (ascorbic acid).

The invention relies on Br⁻ to promote at least one of heart health, vascular health, or kidney health in a subject. Embodiments of the invention promote heat health, vascular health and kidney health in a subject. Embodiments of the invention encompass administering less than 1 g of taurine to a subject. In further embodiments, undetectable amounts of taurine can be administered to a subject. In some embodiments of the invention, the compositions do not contain taurine.

Some nutritional embodiments of the invention comprise a dietary item. Non-limiting examples of dietary items include food products, dietary ingredients, medical foods, functional foods, beverages, dietary supplements, vitamins, minerals, and combinations thereof. Some nutritional embodiments are orally administered to a subject. Some nutritional embodiments are administered to a subject who has cardiovascular disease, hypertension, kidney disease, end stage renal disease, diabetes, metabolic syndrome, obesity, cancer, an eye disease, an ear disease, a wound, an ulcer, an infection, inflammation, a degenerative disease, a skin disease, cystic fibrosis, or a combination thereof.

Embodiments can be used to modulate a biomarker that is associated with heart health, kidney health, or vascular health. In some embodiments, the biomarker is a serum or blood biomarker of heart health, kidney health, or vascular health, where non-limiting examples of such biomarkers include lipids, cholesterol, high-density lipoprotein, low-density lipoprotein, very-low-density lipoprotein, homocysteine, creatinine, c-reactive protein, cystatin C, tryglyceride, lipid oxidation, protein oxidation, kidney injury molecule 1, neutrophil gelatinase-associated lipocalin, systolic or diastolic blood pressure, heart rate, one or more peptides of collagen IV or III, one or more peptides of brain natriuretic peptide (BNP), one or more peptides of atrial natriuretic peptide, one or more peptides of pro-BNP, cyclic guanosine monophosphate, or glomerular filtration rate. In some embodiments, the biomarker is a urine biomarker of heart health, kidney health, or vascular health, where non-limiting examples of such biomarkers include creatinine, protein, albumin, homocysteine, lipid oxidation, protein oxidation, kidney injury molecule 1, neutrophil gelatinase-associated lipocalin, one or more peptides of collagen IV or III, one or more peptides of brain natriuretic peptide (BNP), one or more peptides of atrial natriuretic peptide, one or more peptides of pro-BNP, cyclic guanosine monophosphate, or glomerular filtration rate. In embodiments, the biomarker is selected from one or more of systolic blood pressure that is higher than 120 mm Hg, diastolic blood pressure that is higher than 80 mm Hg, average resting heart rate that higher than 100 beats per minute; glomerular filtration rate less than 60 ml/min/1.73 m2, or urine albumin levels over 30 mg/g. Embodiments of the invention can be used to maintain the levels or concentrations of one or more biomarkers to be within those levels or concentrations that are healthy, normal, baseline, or otherwise medically acceptable. In embodiments, blood levels of natriuretic peptides, such as ANP and BNP can indicate the need for administration of Br-compositions as described herein, where BNP levels below about 50 pg/ml or above 100 pg/ml or where ANP levels above about 77 pg/ml or below about 22 pg/ml can indicate a need for administration of Br-compositions as described herein. In some embodiments, BNP levels below about 40 pg/ml, or below about 30 pg/ml, or even below about 20 pg/ml, or even still below about 10 pg/ml can indicate a need for administration of Br-compositions as described herein. In some embodiments, BNP levels above about 100 pg/ml, or above about 200 pg/ml, or above about 300 pg/ml, or above about 400 pg/ml, or above about 500 pg/ml, or above about 600 pg/ml, or above about 700 pg/ml, or above about 800 pg/ml, or above about 900 pg/ml, or above about 1000 pg/ml, or above about 1100 pg/ml, or above about 1200 pg/ml, or above about 1300 pg/ml, or above about 1400 pg/ml, or above about 1500 pg/ml, or above about 1600 pg/ml, or above about 1700 pg/ml, or above about 1800 pg/ml, or above about 1900 pg/ml, or above about 2000 pg/ml can indicate a need for administration of Br-compositions as described herein. In some embodiments, ANP levels below about 20 pg/ml, or below about 15 pg/ml, or below about 10 pg/ml, or even below about 5 pg/ml, or even below about 1 pg/ml, or even still undetectable amounts of ANP can indicate a need for administration of Br-compositions as described herein. In some embodiments, ANP levels above about 77 pg/ml, above about 80 pg/ml, above about 90 pg/ml, above about 100 pg/ml, above about 110 pg/ml, above about 120 pg/ml, above about 130 pg/ml, above about 140 pg/ml, above about 150 pg/ml, above about 160 pg/ml, above about 170 pg/ml, above about 180 pg/ml, above about 190 pg/ml, above about 200 pg/ml, above about 210 pg/ml, above about 220 pg/ml, above about 230 pg/ml, above about 240 pg/ml, above about 250 pg/ml, above about 260 pg/ml, above about 270 pg/ml, above about 280 above about 290 pg/ml, or even above about 300 can indicate a need for administration of Br-compositions as described herein. Embodiments of the invention can be used to therapeutically treat or restore the levels or concentrations of one or more biomarkers to be within those levels or concentrations that are healthy, normal, baseline, or otherwise medically acceptable.

Some method embodiments of the invention comprise a method of promoting heart health, kidney health, vasculature health, or a combination thereof in a subject, the method comprising identifying a subject in need of a therapeutically or nutritionally effective amount of Br⁻ wherein the subject is identified as in need by displaying at least one of the following: systolic blood pressure that is higher than 120 mm Hg, diastolic blood pressure that is higher than 80 mm Hg, average resting heart rate that higher than 100 beats per minute; glomerular filtration rate less than 60 ml/min/1.73 m2, or urine albumin levels over 30 mg/g; obtaining a dietary item or drug comprising an amount of Br⁻ between about 0.1 mg and about 50 mg; administering an effective amount of the dietary item or drug that is required to raise or maintain the circulating levels of Br⁻ in the subject's blood, serum, or plasma to between about 50 μM and about 1 mM; monitoring the safety of the dietary item or drug by measuring the amount of Br⁻ in the subject's blood, serum, or plasma; and ceasing administration of the dietary item or drug if the blood, serum, or plasma Br⁻ level approaches about 1 mM. In embodiments, the blood, serum, or plasma Br⁻ level in a subject is maintained between 50 μM and 500 μM. In embodiments, the blood, serum, or plasma Br⁻ level is measured on a regular basis while the subject is being administered the food, dietary supplement, or drug, such as but not limited to daily, weekly, every other week, monthly, every other month, one time every three months, or yearly. In embodiments, the blood, serum, or plasma Br⁻ is measured using standard clinical chemistry techniques include column chromatography, inductively coupled plasma mass spectrometry, x-ray florescence, and neutron activation analysis.

Embodiments of the invention can be administered to a subject that is human. In some embodiments, the subject is a patient with heart disease, kidney disease, and/or vascular disease. In some embodiments, the subject is at risk of developing heart disease, kidney disease, and/or vascular disease. In some embodiments, the subject has a family history of heart disease, kidney disease, and/or vascular disease. In some embodiments, the subject is an animal. In some embodiments, the subject is a dog, cat, mouse, rabbit, rat, hamster, guinea pig, horse, cow, goat, sheep, pig, chicken, or turkey.

Embodiments of the invention comprise compositions that are manufactured, processed, formulated, or designed to contain a nutritionally effective amount of bromide salts (Br⁻). For some embodiments, the source of Br⁻ can be a standard food item, a purified food, or a chemical with acceptable use as a dietary or pharmaceutical ingredient. Non-limiting examples of the embodiments include food products, dietary ingredients, medical foods, drugs, functional foods, beverages, dietary supplements, vitamins, minerals, and combinations thereof. In some embodiments, the invention is distributed for consumption by humans. In other embodiments, the invention is distributed for consumption by one or more animals, such as but not limited to a dog, cat, mouse, rabbit, rat, hamster, guinea pig, horse, cow, goat, sheep, pig, chicken, or turkey. In some embodiments, a nutritionally effective amount of Br⁻ is combined with one or more dietary ingredients, foods, fluids, vitamins, supplements, or minerals. In some embodiments, the invention can be prepared as one or more pills, liquids, capsules, chews, or dissolvable strips.

In some embodiments of the invention for human use, a nutritionally effective amount of Br⁻ comprises an amount between about 0.1 and 50 mg of Br⁻ that is administered to a subject. In some embodiments, a nutritionally effective amount of Br⁻ is administered to a subject within a single 24 hour period. In some embodiments, the invention is administered to a subject one time per day. For example, the embodiment can be consumed in the morning, mid-day, evening, or other time of the day with or without food. In other embodiments, the invention is administered multiple times per day to a subject, where the total amount of Br⁻ consumed daily amounts to an amount between about 0.1 and 50 mg Br⁻. For example, the embodiment can be administered as a morning and afternoon snack where an individual snack contains 0.75 mg of Br⁻ for a total of 1.5 mg of Br⁻ consumed daily. Alternatively, the embodiment can be administered with every meal where 1 mg of Br⁻ is administered alongside three regular meals for a total of 3 mg Br⁻ being administered within a single day.

In some embodiments of the invention for human or animal use, a nutritionally effective amount of Br⁻ comprises a daily consumption of an amount between about 0.1 mg-50 mg Br⁻. In some embodiments, the exact amount of administered Br⁻ can be adjusted to specific circumstances surrounding the subject. For example, some subjects with kidney disease may be administered less than 20 mg Br⁻ per day, less than 10 mg Br⁻ per day, or even less than 1 mg Br⁻ per day. As another example, other subjects with kidney disease may have greater amounts of Br⁻, perhaps due to increased urinary excretion of Br⁻, with non-limiting examples of said amounts comprising greater than about 20 mg Br⁻ per day, greater than about 30 mg Br⁻ per day, or between about 40 mg and about 50 mg Br⁻ per day. Alternatively, subjects who regularly consume a high salt diet may be administered more than 1 mg Br⁻ per day, more than 10 mg Br⁻ per day, or even more than 20 mg Br⁻ per day. The actual amount of Br⁻ taken daily can be determined by a doctor, nutritionist, or an informed consumer.

Embodiments of the invention can be repeatedly administered to a subject. For example, the invention can be administered daily, every other day, every third day, twice per week, three times per week, weekly, or monthly to a subject. In some embodiments, the invention is administered to a subject while said subject is undergoing a specific medical regimen, such as maintenance dialysis. In other embodiments, the invention is prophylactically administered to promote the heart health, kidney health, and/or vascular health in a subject. For example, said subject may be at risk of developing one or more forms of cardiovascular disease, kidney disease, or vascular disease. As another example, said subject may be an adult at risk of developing one or more age-related forms of cardiovascular disease, kidney disease, or vascular disease.

In other embodiments, the invention comprises methods for identifying a subject in poor heart health, kidney health, or vascular health. In some embodiments, the subject can be in poor health if (1) serum or blood Br⁻ levels are between about 20 μM and 50 μM; (2) the subject is being treated with dialysis; (3) the subject is being treated with a diuretic; (4) dietary Na⁺ consumption is above about 2300 mg per day; (5) dietary Cl⁻ consumption is above about 3545 mg per day.

In embodiments of the invention, Br⁻ levels can be measured in biological samples, such as biological fluids. Non-limiting examples of biological fluids include saliva, blood, serum, spent or used dialysate, sweat, or urine. Such measurements can be made using techniques known to the art. Non-limiting examples of such standard clinical chemistry techniques include column chromatography, inductively coupled plasma mass spectrometry, x-ray florescence, and neutron activation analysis.

Embodiments of the invention comprise identifying a subject who is in need of a nutritionally effective amount of Br⁻, whereby the subject is in need if Br⁻ levels in blood or serum are at or below about 50 μM, or if urinary excretion rates of Na⁺ and/or Cl⁻ are elevated above the normal range for that patient or above what is otherwise considered a healthy range. In some embodiments, the subject may display a preferred response to practice of the invention if Br⁻ levels in blood or serum are at or below about 50 μM, or if urinary excretion rates of Na⁺ and/or Cl⁻ are elevated above the normal range for that patient or above what is otherwise considered a healthy range. In some embodiments, the invention comprises a method for the diagnostic identification of those subjects who are in need of a nutritionally effective amount of Br⁻. Other embodiments of the invention comprise identifying a subject who is Br-deficient. For example, the invention can be useful in preventing or treating Br-deficiency in people and animals.

In embodiments of the invention, Br⁻ levels can be analytically measured in a dietary item. For example, Br⁻ can be measured in a dietary item during manufacturing in order to determine whether the dietary item contains a nutritionally effective amount of Br⁻. Non-limiting examples of such analytical techniques include column chromatography, inductively coupled plasma mass spectrometry, x-ray florescence, and neutron activation analysis.

In another embodiment, the invention comprises a dietary item containing a nutritionally effective amount of Br⁻. In some embodiments, the invention is used by a subject with cardiovascular disease or kidney disease. In other embodiments, the invention is used by a subject in poor heart health, kidney health, and/or vascular health. In some embodiments, the dietary item contains about 0.1 mg, 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 20.5 mg, 21.0 mg, 21.5 mg, 22.0 mg, 22.5 mg, 23.0 mg, 23.5 mg, 24.0 mg, 24.5 mg, 25.0 mg, 25.5 mg, 26.0 mg, 26.5 mg, 27.0 mg, 27.5 mg, 28.0 mg, 28.5 mg, 29.0 mg, 29.5 mg, 30.0 mg, 30.5 mg, 31.0 mg, 31.5 mg, 32.0 mg, 32.5 mg, 33.0 mg, 33.5 mg, 34.0 mg, 34.5 mg, 35.0 mg, 35.5 mg, 36.0 mg, 36.5 mg, 37.0 mg, 37.5 mg, 38.0 mg, 38.5 mg, 39.0 mg, 39.5 mg, 40.0 mg, 40.5 mg, 41.0 mg, 41.5 mg, 42.0 mg, 42.5 mg, 43.0 mg, 43.5 mg, 44.0 mg, 44.5 mg, 45.0 mg, 45.5 mg, 46.0 mg, 46.5 mg, 47.0 mg, 47.5 mg, 48.0 mg, 48.5 mg, 49.0 mg, 49.0 mg, 49.5 mg, 50.0 mg, or any other amount between about 0.1 mg and about 50.0 mg of Br⁻. The dietary item can be prepared as a purified or synthesized dietary item. Non-limiting examples of dietary items include food products, dietary ingredients, medical foods, functional foods, beverages, dietary supplements, vitamins, minerals, and combinations thereof. The dietary item may be supplemented with Br⁻ by combining a standard food item with a purified food or other source of Br⁻ so that the final form of the dietary item contains a nutritionally effective amount of Br⁻.

In embodiments, the dietary item can be a solid, such as but not limited to a snack or meal, that contains a nutritionally effective amount of Br⁻. In one embodiment, the dietary item is a food item comprising Br⁻. For example, the dietary item can be a food item containing between about 0.1 mg and 50 mg of Br⁻ per serving. Embodiments of the invention can be food items that promote the health of the heart, kidneys, and/or vasculature in a subject. Embodiments of the invention can comprise a nutritionally effective amount of Br³¹ . Other embodiments of the invention can be prepared as either a purified or synthesized item. In other embodiments, the item can be prepared as seen with parenterally-administered or enterally-administered food items. In still other embodiments, the food can be a composition comprising a standard food item and Br⁻ so that the composition comprises the amount of Br⁻ required.

In one embodiment, the dietary item can be a fluid containing a nutritionally effective amount of Br³¹ . Embodiments of the invention can be products that promote the health of the heart, kidneys, and/or vasculature in a subject. In some embodiments, the fluid composition can be formulated as a liquid dietary supplement, beverage, drink, shake, juice, or smoothie. In embodiments, the fluid contains 0.1 mg, 0.5 0 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 20.5 mg, 21.0 mg, 21.5 mg, 22.0 mg, 22.5 mg, 23.0 mg, 23.5 mg, 24.0 mg, 24.5 mg, 25.0 mg, 25.5 mg, 26.0 mg, 26.5 mg, 27.0 mg, 27.5 mg, 28.0 mg, 28.5 mg, 29.0 mg, 29.5 mg, 30.0 mg, 30.5 mg, 31.0 mg, 31.5 mg, 32.0 mg, 32.5 mg, 33.0 mg, 33.5 mg, 34.0 mg, 34.5 mg, 35.0 mg, 35.5 mg, 36.0 mg, 36.5 mg, 37.0 mg, 37.5 mg, 38.0 mg, 38.5 mg, 39.0 mg, 39.5 mg, 40.0 mg, 40.5 mg, 41.0 mg, 41.5 mg, 42.0 mg, 42.5 mg, 43.0 mg, 43.5 mg, 44.0 mg, 44.5 mg, 45.0 mg, 45.5 mg, 46.0 mg, 46.5 mg, 47.0 mg, 47.5 mg, 48.0 mg, 48.5 mg, 49.0 mg, 49.0 mg, 49.5 mg, 50.0 mg, or any other amount between about 0.1 mg and about 50.0 mg of Br³¹ . In some embodiments, the fluid can be flavored, flavorless, carbonated, bottled, or concentrated. In some embodiments, the invention can be made from a powder, tablet, or solid that contains a nutritionally effective amount of Br⁻ and is dissolved in a fluid prior to being consumed.

In one embodiment, the invention comprises a table salt comprising a mixture of Br⁻ and Cl⁻ ions. In embodiments, the table salt comprises a molar amount of Br⁻ ions that is between about 0.0338% and about 4.613% of the molar amount of Cl⁻. In another embodiment, the invention is an iodized table salt. In another embodiment, the table salt is useful as a salt substitute. In another embodiment, the table salt can be used as a food ingredient. In yet another embodiment, the table salt can be useful for food preparation, seasoning, and/or preservation. In still another embodiment, the table salt is marketed for use by individuals with poor heart health, poor kidney health, poor vascular health, cardiovascular disease, kidney disease, or a combination thereof.

In one embodiment, the invention comprises adding Br⁻ to a dietary item. In embodiments, the final molar amount of Br⁻ ions in the dietary item are between about 0.0338% and about 4.613% of the molar amount of Cl⁻ ions on a molar basis. For example, said addition step can be performed by adding a sufficient amount of Br⁻ ions to table salt, so that the final molar amount of Br⁻ is between about 0.0338% and about 4.613% of the molar amount of Cl⁻. As another example, said addition step can be performed by first adding a sufficient amount of Br⁻ ions to a food product, then adding a sufficient amount of table salt or other chloride-containing item so that the final molar amount of Br⁻ in the finished dietary item is between about 0.03381% and about 0.9659% relative to the molar amount of Cl⁻. In some embodiments, the final molar amount of Br⁻ in the finished dietary item is between about 0.03381% and about 4.613% relative to the molar amount of Cl⁻.

In yet another embodiment, the invention comprises a medical food that is supplemented to provide subjects with a nutritionally effective amount of Br³¹ . In one embodiment, the medical food is used to treat or prevent Br⁻ deficiency in a subject. For example, the medical food is supplemented with Br⁻ to provide a subject with an amount between about 0.1 mg and 50 mg of Br⁻ per day. Medical foods can be consumed or enterally administered to subjects under doctor supervision. For example, subjects receiving dialysis experience treatment-induced loss of Br⁻. As such, embodiments of the invention can be administered under doctor supervision as a food or drink product to subjects on dialysis immediately after each dialysis treatment and before their next regular meal or snack.

In yet another embodiment, the invention comprises a functional food that promotes the health of a subject. For example, the food can promote at least one of heart health, kidney health, and/or vascular health in a subject. In some embodiments, the functional food is made by enriching a standard food, dietary ingredient, or beverage with a nutritionally effective amount of Br³¹ .

Compositions of the invention as described herein can be administered to specific groups of subjects that are at risk of developing medical complications involving the heart, kidney, or vasculature Compositions of the invention as described herein can be administered to subjects undergoing dialysis treatments. Other embodiments of the invention can be administered to subjects in a hospital. Still other embodiments of the invention can be administered to individuals who consume a high salt or high sodium diet. Compositions of the invention as described herein can be administered to subjects who receive their meals under the supervision of a nutrition specialist or physician, such as subjects receiving enteral or parental nutrition.

Some embodiments of the invention comprise a method of preventing cardiovascular disease or kidney disease in a subject, the method comprising administering to the subject a nutritiously effective amount of a dietary item containing a molar amount of Br⁻ ions between about 0.0338% and about 4.613% of the molar amount of Cl⁻. In some embodiments, the circulating Br⁻ level in the serum, blood, or plasma of the subject is at or below 20 μM. In some embodiments, administration of the dietary item maintains circulating Br⁻ levels in the subject to above about 50 μM and below about 500 μM.

Some embodiments of the invention can be administered to a subject at risk of developing one or more forms of cardiovascular disease, kidney disease, or vascular disease. In some embodiments, the subject has a history or family history of one or more forms of cardiovascular disease, kidney disease, or vascular disease. In other embodiments, non-limiting forms of disease include chronic kidney disease, end stage renal disease, glomerular disease, tubular disease, kidney injury, acute kidney injury, sepsis-induced kidney injury, drug-induced kidney injury, hypovolemia-induced kidney injury, ischemic kidney injury, fibrosis, vascular disease, hypertension, salt-sensitive hypertension, heart attack, heart failure, cardiac remodeling, cardiac fibrosis, myocardial fibrosis, atherosclerosis, stroke, arterial stiffening, vascular wall thickening, thickening of the peritoneal membrane, dyslipidemia, blood clot, anemia, an acid-base imbalance, hypercholemia, infection, sepsis, thrombosis, coronary artery disease, ischemic heart disease, peripheral artery disease, or any combination thereof.

In yet another embodiment, embodiments can comprise a dietary item for animalsthat comprises a nutritionally effective amount of Br⁻. In an embodiment, the dietary item can contain an amount between about 0.1 mg and about 250 mg Br⁻ per kg of food, administering a total amount of between about 0.1 and about 50 mg Br⁻ to the animal per day, or contains between about 0.0338% and about 0.9659% Br⁻ relative to the amount of Cl⁻ on a molar basis. In another embodiment, the dietary item can contain an amount between about 0.1 and about 250 mg Br⁻ per kg of food, or contains between about 0.0338% and about 4.613% Br⁻ relative to the amount of Cl⁻ on a molar basis. Some embodiments of the invention can be administered to cats that have or are at risk of developing feline cardiomyopathy. Other embodiments of the invention can be administered to dogs who have are at risk of developing canine kidney disease. Still other embodiments of the invention can be used to promote heart health, kidney health, and/or vascular health.

In yet another embodiment, embodiments can be combined with foods, beverages, devices, or drugs that reverse, prevent, or treat nutritional or vitamin deficiencies. Non-limiting examples include deficiencies in magnesium, vitamin C, one or more of the B vitamins, iron, or deficiencies in other vitamins and minerals important for heart, kidney, and vasculature health. In some embodiments of the invention, a nutritionally effective amount of Br⁻ can be co-administered to a subject along with one or more of these vitamins and minerals.

Embodiments of the invention comprise a method of administering to a subject a dietary item that is beneficial for said subject's heart, kidneys, or vasculature, the method comprising (1) measuring the amount of ionic Br⁻ in a dietary item; (2) predicting that the dietary item is beneficial for heart health, kidney health, and/or vascular health if the dietary item contains a nutritionally effective amount of ionic Br⁻, and (3) administering the dietary item to a subject in need thereof Embodiments of this method can comprise measuring the amount of ionic Br⁻ in a representative dietary item. Non-limiting examples of dietary items include food products, dietary ingredients, medical foods, functional foods, beverages, dietary supplements, vitamins, minerals, and combinations thereof.

Nutritional composition embodiments can be used to help a subject maintain one or more biomarkers within a healthy range. In some embodiments, the biomarker and its healthy level or range is selected from serum, blood, or plasma Br-concentration between about 30 μM and about 1 mM; systolic blood pressure between about 90 mm Hg and about 120 mm Hg; diastolic blood pressure between about 60 mm Hg and about 80 mm Hg; GFR above about 60; resting heart rate between about 60 and about 100 beats per minute; and urinary albumin levels below about 30 mg/g.

Without being bound by theory, embodiments of this method can identify a subject in need of a dietary item that is beneficial for heart health, kidney health, and or vascular health by (1) using a diagnostic test to measure the serum, blood, salivary, or urinary biomarkers levels of lipids, cholesterol, high-density lipoprotein, low-density lipoprotein, very-low-density lipoprotein, homocysteine, creatinine, c-reactive protein, cystatin C, tryglyceride, protein, albumin, lipid oxidation, protein oxidation, kidney injury molecule 1, or neutrophil gelatinase-associated lipocalin; (2) for each biomarker examined, comparing the test result to the healthy range of said biomarker levels for the subject; (3) for each biomarker examined, calculating the absolute difference between the test result and the upper and lower boundaries of the healthy range; (4) for each biomarker examined, calculating the difference between the upper boundary and the lower boundary of the healthy range; (5) for each biomarker examined, calculating the ratio of the absolute difference found in Step 3 to the difference found in Step 4; and (6) for each biomarker examined, determining that the subject is in said poor health when the ratio calculated in Step 5 is less than or about 0.20. The two equations below illustrate the calculations described in Steps 2-6, where “Result” is the test result determined in Step 1, “Up” is the upper boundary of the healthy range of the biomarker of interest, and “Low” is the lower boundary of the healthy range of the biomarker of interest:

$$\frac{⌈ Test - Up ⌉}{( Up - Low )} \leq 0.20$$

$$or$$

$$\frac{⌈ Test - Low ⌉}{( Up - Low )} \leq 0.20$$

In some embodiments, the invention comprises a dietary item beneficial or healthy for heart, kidney, or vasculature when there is a nutritionally effective amount of Br⁻ ions in the item.

Embodiments of the invention can be used by the general public. Embodiments can be used by individuals who are considered healthy, and who would like to remain healthy. Other embodiments can be administered to specific demographic segments or patient populations, such as but not limited to hospital patients; individuals with end-stage renal disease, cardiovascular disease, or kidney disease; adult, elderly, or geriatric populations; or individuals receiving chemotherapeutic or hypertensive therapeutic drugs.

Embodiments of the invention can be used as drugs. Some embodiments may treat, diagnosis, or prevent heart disease, kidney disease, or vascular disease in a subject, and may thus be considered drugs. In some embodiments, a nutritionally effective amount of Br⁻ is administered to a subject via a drug.

Some embodiments of the invention comprise a dietary supplement for patients with kidney disease or cardiovascular disease. In embodiments, the dietary supplement comprises between about 0.1 mg and about 50 mg of bromide (Br⁻). In one embodiment, the sodium bromide is used as the source of bromide. In another embodiment, potassium bromide is used as the source of bromide. In still another embodiment, magnesium bromide is used as the source of bromide.

In another embodiment, the invention comprises a method of promoting the health of the heart, kidneys, and/or vasculature in a subject by administering an effective amount of bromide (Br⁻) to the subject. In some embodiments of invention, the health of a subject is promoted by modifying the structure or function of the heart, kidney, and/or vasculature. In some embodiments, an amount between about 0.1 mg and about 50 mg of bromide (Br⁻) is administered to the subject. In other embodiments, an amount between about 0.1 mg and about 1 mg of bromide (Br) is administered to the subject. In other embodiments, an amount between about 1 mg and about 5 mg of bromide (Br^{—}) is administered to the subject. In still other embodiments, an amount between about 8 mg and about 12 mg of bromide (Br⁻) is administered to the subject. In still other embodiments, an amount between about 13 mg and about 20 mg of bromide (Br⁻) is administered to the subject. In yet other embodiments, an amount between about 20 mg and about 25 mg of bromide (Br⁻) is administered to the subject. In other embodiments, an amount between about 25 mg and about 50 mg of bromide (Br⁻) is administered to the subject. In some embodiments, the subject is an animal. In other embodiments, the subject is any one of a human, dog, cat, mouse, rabbit, rat, hamster, guinea pig, horse, cow, goat, sheep, pig, chicken, or turkey.

In some embodiments, the invention comprises a table salt comprising a mixture of Br⁻ and Cl⁻ ions where the molar amount of Br⁻ ions is between about 0.0338% and about 4.613% of the molar amount of Cl⁻. In some embodiments, the salt is a food ingredient. In some embodiments, the salt is iodized. In some embodiments, the salt is used for food preparation, seasoning, and/or preservation.

Some embodiments of the invention comprise a method of manufacturing a dietary item. In embodiments, the dietary item benefits heart, kidney, or vascular health. In embodiments, the method comprises adding a bromide salt (Br⁻) or a Br-rich ingredient to a food product. In some embodiments, an amount between about 0.1 mg and about 25 mg of Br⁻ is added to a serving of the dietary item. In some embodiments, the dietary item comprises a food product, dietary ingredient, medical food, functional food, beverage, dietary supplement, vitamin, mineral, and combination thereof.

In some embodiments, the invention comprises a nutritional composition comprising Br⁻, wherein the composition promotes heart health, kidney health, vascular health, or a combination thereof in a subject. In some embodiments, the composition comprises between about 0.1 mg and about 50 mg Br³¹ . In some embodiments, the composition further comprises magnesium, vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid), vitamin B₁₂ (cobalamin), vitamin C (ascorbic acid), vitamin E (tocopherol), selenium, zinc, iron, vitamin D, or a combination thereof. In some embodiments, the composition comprises less than about 50 mg taurine. In some embodiments, the subject comprises an animal. In some embodiments, the animal comprises a human, dog, cat, mouse, rabbit, rat, hamster, guinea pig, horse, cow, goat, sheep, pig, chicken, or turkey. In some embodiments, the composition is orally administered to the subject. In some embodiments, the subject has cardiovascular disease, hypertension, kidney disease, end stage renal disease, cancer, or a combination thereof. In some embodiments, the composition is a dietary supplement or drug.

In some embodiments, the invention comprises a table salt comprising Br⁻ and Cl⁻ ions, the amount of Br⁻ are an amount between about 0.0338% and about 4.613% of the amount of Cl⁻ on a molar basis. For example, some embodiments comprise an amount of Br⁻ that is about 0.0338%, 0.04%, 0.05%, 0.06%, 0.07%, 0.08%, 0.09%, 0.10%, 0.12%, 0.14%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, 1.00%, 1.02%, 1.04%, 1.06%, 1.08%, 1.10%, 1.12%, 1.14%, 1.16%, 1.18%, 1.20%, 1.22%, 1.24%, 1.26%, 1.28%, 1.30%, 1.32%, 1.34%, 1.36%, 1.38%, 1.40%, 1.42%, 1.44%, 1.46%, 1.48%, 1.50%, 1.52%, 1.54%, 1.56%, 1.58%, 1.60%, 1.62%, 1.64%, 1.66%, 1.68%, 1.70%, 1.72%, 1.74%, 1.76%, 1.78%, 1.80%, 1.82%, 1.84%, 1.86%, 1.88%, 1.90%, 1.92%, 1.94%, 1.96%, 1.98%, 2.00%, 2.02%, 2.04%, 2.06%, 2.08%, 2.10%, 2.12%, 2.14%, 2.16%, 2.18%, 2.20%, 2.22%, 2.24%, 2.26%, 2.28%, 2.30%, 2.32%, 2.34%, 2.36%, 2.38%, 2.40%, 2.42%, 2.44%, 2.46%, 2.48%, 2.50%, 2.52%, 2.54%, 2.56%, 2.58%, 2.60%, 2.62%, 2.64%, 2.66%, 2.68%, 2.70%, 2.72%, 2.74%, 2.76%, 2.78%, 2.80%, 2.82%, 2.84%, 2.86%, 2.88%, 2.90%, 2.92%, 2.94%, 2.96%, 2.98%, 3.00%, 3.02%, 3.04%, 3.06%, 3.08%, 3.10%, 3.12%, 3.14%, 3.16%, 3.18%, 3.20%, 3.22%, 3.24%, 3.26%, 3.28%, 3.30%, 3.32%, 3.34%, 3.36%, 3.38%, 3.40%, 3.42%, 3.44%, 3.46%, 3.48%, 3.50%, 3.52%, 3.54%, 3.56%, 3.58%, 3.60%, 3.62%, 3.64%, 3.66%, 3.68%, 3.70%, 3.72%, 3.74%, 3.76%, 3.78%, 3.80%, 3.82%, 3.84%, 3.86%, 3.88%, 3.90%, 3.92%, 3.94%, 3.96%, 3.98%, 4.00%, 4.02%, 4.04%, 4.06%, 4.08%, 4.10%, 4.12%, 4.14%, 4.16%, 4.18%, 4.20%, 4.22%, 4.24%, 4.26%, 4.28%, 4.30%, 4.32%, 4.34%, 4.36%, 4.38%, 4.40%, 4.42%, 4.44%, 4.46%, 4.48%, 4.50%, 4.52%, 4.54%, 4.56%, 4.58%, 4.60%, about 4.613%, or any other amount that is between about 0.0338% and about 4.613% of the amount of Cl⁻ on a molar basis. In some embodiments, the table salt is iodized. In other embodiments, the table salt is non-iodized, kosher, or pickling salt. In some embodiments, the table salt promotes heart health, kidney health, vascular health, or a combination thereof in a subject. In some embodiments, the salt is iodized. In some embodiments, the salt is used for food preparation, seasoning, and/or preservation.

Some embodiments of the invention comprise a method of manufacturing a dietary item to promote heart health, kidney health, vascular health, or a combination thereof, the method comprising adding a nutritionally effective amount of bromide (Br⁻) to the item. In some embodiments, the nutritionally effective amount of Br⁻ comprises between about 0.1 mg and about 50 mg of Br³¹ . In some embodiments, the dietary item comprises a food product, dietary ingredient, medical food, drug, functional food, beverage, dietary supplement, vitamin, mineral, or combination thereof.

Embodiments of the invention comprise method of promoting heart health, kidney health, vasculature health, or a combination thereof in a subject, the method comprising: (1) identifying a subject in need thereof, wherein the subject is identified as in need when the serum Br⁻ level is at or below 20 μM; (2) determining the amount of ionic bromide (Br⁻) needed to raise the circulating levels of Br⁻ in the subject to physiologically normal levels; (3) preparing a dietary item or drug comprising only the amount of Br⁻ necessary to raise the circulating levels of Br⁻ in the subject to physiologically normal levels; and (4) administering to the subject the dietary item or drug, wherein the dietary item or drug promotes heart health, kidney health, vasculature health, or a combination thereof. In some embodiments, the dietary item comprises between about 0.1 mg and about 50 mg of ionic bromide (Br⁻). In some embodiments, the circulating levels of Br⁻ in the subject comprise serum Br⁻ concentration of about 30 μM or less.

Some embodiments of the invention comprise a method of preventing cardiovascular disease or kidney disease in a subject, the method comprising measuring the Br⁻ concentration in the serum of a subject, determining the subject to be at risk of developing cardiovascular disease or kidney disease if the Br⁻ serum concentration is below 20 μM, and administering a nutritiously effective amount of Br⁻ to the subject to prevent cardiovascular disease or kidney disease, wherein the nutritiously effective amount of Br⁻ is administered by a dietary item or drug.

In some embodiments, the invention comprises a method for the treatment of disease in a subject, said method comprising administering to a subject in need of such treatment a nutritiously effective amount of Br⁻ in an amount no greater than that required to result in a body fluid concentration no greater than 500 μM, wherein the nutritiously effective amount of Br⁻ is administered by a dietary item or drug. In some embodiments, the disease comprises a disease of the circulatory system, a cardiovascular disease, a kidney disease, or a combination thereof.

In some embodiments, the invention comprises a composition comprises an amount of Br⁻ no greater than that required to result in a body fluid concentration of Br⁻ no greater than 1 mM in a subject, wherein the composition is formulated as a dietary item or drug to be ingested by the subject. In some embodiments, the composition promotes heart health, kidney health, vasculature health, or a combination thereof.

Some embodiments comprise administering doses of Br⁻ at differing concentrations over a period of time in order to achieve the desired circulating Br⁻ level in the blood, serum, or plasma of a subject. For example, a subject can be administered a composition comprising a high dose of Br⁻, such as but not limited to about 15 mg to about 25 mg Br⁻, followed by subsequent administration of lower doses of Br⁻, such as but not limited to about 1 mg to about 5 mg Br⁻ a period time thereafter. This approach can benefit a subject who has serum Br⁻ levels below 20 where the initial large dose serves to restore serum Br⁻ levels to between about 50 μM and about 500 μM while the subsequently administered lower doses of Br⁻ serve to maintain the serum Br⁻ levels in this range. Another non-limiting example includes administering compositions comprising increasing doses of Br⁻ to a subject until a desired maintenance dosing is achieved, such as but not limited to increasing dosing from about 1 mg Br⁻ to about 3 mg Br⁻ to about 5 mg Br⁻ and so on until a maintenance dose of about 10 mg Br⁻ is achieved. Still another non-limiting example includes administering a daily maintenance dose of about 5 mg Br⁻ with periodic injections of larger doses such as but not limited to about 10 mg Br⁻, about 15 mg Br⁻, about 20 mg Br⁻, or about 25 mg Br³¹ .

Importantly, in order to prevent toxicity, embodiments as described herein do not provide for administering sufficient amounts of Br⁻ to raise the circulating Br⁻ concentration above 1 mM in a subject, particularly if two or more separate compositions, each comprising Br—, are administered to the same subject. As a non-limiting example, if a subject is administered a combination of two or more drug formulations each containing Br—, for example a first drug for controlling diabetes combined with a second drug for controlling hypertension, it would be out of scope of the invention for the subject's blood concentration of Br⁻ to rise above 1 mM. Thus, embodiments as described herein comprise only administering sufficient Br⁻ to raise and maintain circulating Br⁻ concentration above at least 20 μM Br⁻ but not more than 1 mM. Further embodiments administer sufficient Br⁻ to achieve circulating Br⁻ concentrations above 50 μM but below 500 μM Br³¹ .

### BRIEF DESCRIPTION OF THE FIGURES

FIG. 1 shows that Br⁻ significantly increases survival in salt sensitive Dahl rats. (FIG. 1A) Dahl rats on AIN76A+8% NaCl display significantly lower serum Br⁻ levels compared to Dahl rats on AIN76A diet (mean: 14.14 μM vs. 25.54 μM, respectively; *, p<0.001, T-test), indicating that an important threshold for inducing symptoms of Br-deficiency occurs at or near 20 μM. High salt in known to induce symptoms of cardiovascular disease and kidney disease in Dahl rats, in this study, mortality events appeared after rats were on a high salt diet for approximately one month. *:p=0.0001, T-test. (FIG. 1B) Rats were administered a diet of AIN76A+8% NaCl with or without 300 mg NaBr per kg feed (“High Salt+Br” and “High Salt” study arms, respectively). Control rats received AIN76A without supplemental chloride or bromide. 9 rats per study arm. Significantly greater survival probability need in High Salt+Br compared to High Salt alone (log rank test), demonstrating ability of Br⁻ to improve preclinical mortality rates caused by high salt diet. **:p<0.001, Log-Rank.

FIG. 2 shows evidence of safe administration of embodiments of the invention in repeat dosing studies in rats. (FIG. 2A) Serum Br⁻ concentrations from rats fed a daily diet over 10 weeks of “AIN-76A”, “AIN-76A+8% NaCl”, “AIN-76A+Br”, or “AIN-76A+8% NaCl+Br”. Salt sensitive Dahl rats as well as Spontaneously Hypertensive Rats (SHR) were used in these studies. The two diets containing 8% NaCl (eg. “AIN-76A+8% NaCl” and “AIN-76A+8% NaCl+Br”) were fed to Dahl rats while the remaining two diets (eg. “AIN-76A” and “AIN-76A+Br”) were fed to SHR, as indicated in panel A. Serum Br⁻ measurements were determined using mass spectrometry. Importantly, both Br⁻ treatments (eg. “AIN-76A+Br” nor “AIN-76A+85 NaCl+Br”) resulted in non-toxic serum Br⁻ concentrations, indicating that embodiments of the invention can safety harness the therapeutic potential of Br³¹ . (FIG. 2B) Actual Br-levels (final) were determined in AIN-76A, AIN-76A+8% NaCl, AIN-76A+Br, or AIN-76A+8% NaCl+Br diets using epiboron instrumental neutron activation analysis. (FIG. 2C) Embodiments of the invention administer safe amounts of Br⁻ to subjects, targeting those circulating Br⁻ concentrations that exhibit no observed adverse effects, which distinguishes these embodiments from other Br-containing drugs that raise circulating Br⁻ concentrations to toxic levels. Not to scale.

FIG. 3 demonstrates the occurrence of low Br⁻ concentrations in Br-Deficient medical salines. Two commercially-available Br-deficient saline solutions (0.9% NaCl) were analyzed for Br-content using epiboron neutron activation analysis. The samples, termed Saline 1 and Saline 2, displayed 15.27 μM Br— and 12.39 μM Br⁻, respectively. Both of these detected concentrations are below the lowest concentration of Br⁻ in embodiments of the invention. Moreover, both Br-deficient salines are intended for topical administration to wounds, which if used as intended, would expose the treated wound to an imbalanced amount of Br⁻ and Cl⁻ ions.

FIG. 4 shows target ratios of Br⁻ to Cl⁻. These mass ratios are calculated from the desired amount of Br⁻ and Cl⁻ ions in the final composition.

FIG. 5 shows daily Br⁻ administration in 10 week preclinical study using SHR disease model. (FIG. 5 A-B) Heart and kidney weights from 10 week repeat dosing study in SHR model. Br⁻ administered daily via diet, with AIN-76a as base diet. WKY served as genetic control. Both kidneys per animal weighed together. Statistically significant reduction in heart weight noted in SHR receiving Br⁻ compared to SHR on AIN-76a alone. *:p<0.05, T-test. (FIG. 5C-D) Heart and kidney weights from panels A-B normalized to body weight. Trends observed in absolute weights are preserved in normalized organ weights. (FIG. 5E) Rats were administered a diet of AIN76A with or without 10 mg NaBr per kg feed (“SHR” and “SHR+Br” study arms, respectively). Survival probability was calculated between SHR rats receiving dietary Br— and SHR rats receiving AIN76A control diet over a 10 week study period. As a second control, Wistar Kyoto (WKY) rats were fed AIN76A control diet over the same period. 8 rats per study arm. One SHR control rat died during a surgical procedure. However, no statistical difference in survival was detected among the groups. (FIG. 5F) After one day on study diet, Br⁻ administered rats displayed significantly reduced urinary microprotein/creatinine ratio.

FIG. 6 shows daily Br⁻ administration reduces cardiac interstitial fibrosis staining. (A) Male Spontaneously Hypertensive Rats (SHR) and (B) salt sensitive Dahl rats on a high salt diet (8% NaCl) receiving daily orally-administered Br⁻ for 10 weeks exhibited less cardiac fibrosis compared to age- and sex-matched SHR controls. AIN-76A was base diet for all animals. Br-supplemented diets formulated as 10 mg NaBr per kg AIN-76a (SHR+Br) and 300 mg NaBr per kg AIN-76a+8% NaCl (Dahl High Salt+Br). Fibrosis determined from 5 μm tissue sections stained with Masson's Trichrome. Fibrotic area per high powered field (40×) quantified using an ImageJ macro (Kennedy et al, Hypertension, 2006,47:488-495) that calculates the percent area where blue staining (collagen) is at least 120% compared to the amount of red staining. Approximately 25-30 images were collected and processed per animal. In total, n=206 images (SHR), 226 images (SHR+Br), 85 images (Dahl High Salt), and 108 images (Dahl High Salt+Br). *p<0.0001, t test.

FIG. 7 show that bromide rapidly reduced blood pressure in SHR. (A) In 10 week-old male SHR, single administration of Br⁻ reduced systolic and diastolic blood pressures compared to saline control. (B) Br⁻ induced a possible reduction of heart rate, but the change did not cross a key threshold for statistical significance (p=0.052, T test). (C-D) Measured values for systolic (C) and diastolic (D) blood pressure, denoting statistical significance where achieved. 154.5 μg NaBr per kg rat body weight injected via IP. n=8 in Br Treatment group, =5 in saline control. BP & HR measured via tail cuff. *:p<0.05, T test.

FIG. 8 show that bromide increases BNP concentration. In 10 week-old male SHR, single administration of Br⁻ significantly modulated the serum concentration of BNP peptide, as measured by ELISA assay. 154.5 μg NaBr per kg rat body weight injected via IP. n=8 in Br Treatment group, =5 in saline control. Baseline serum collection occurred two days prior to treatment. *: p<0.05, T test.

### DETAILED DESCRIPTIONS OF THE INVENTION

### Abbreviations and Definitions

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The terms for “bromine” and “bromide” are often used interchangeably, even in some scientific or medical publications, and can be a source of confusion with respect to the invention. The term “bromine” is used herein in reference to the element; the term “bromide” refers to the ionic specie (Br⁻). “Bromide”, when used herein by itself, does not refer to a molecule that contains a bromine atom that is covalently bound to the parent molecule. An example of such covalently-bound molecules is methyl bromide. Alternatively, the term “brominated” molecule may be used to refer to a molecule that contains a covalently-bound bromine atom.

As used herein, a therapeutically effective amount of Br⁻ can be, for example, the amount of Br⁻ or Br-comprising composition, needed to raise the circulating levels of Br⁻ to those concentrations that are required for eliciting the desired biological response following administration. For example, in a subject with cardiovascular disease and with a serum Br⁻ concentration of about 15 μM, the desired biological response can be treating cardiovascular disease and a therapeutically effective amount of Br⁻ can be the amount of Br⁻ needed to raise the serum Br⁻ to an amount above about 20 μM but below about 1 mM, for example to an amount between about 50 μM to about 1 mM.

As used herein, a nutritionally effective amount of Br⁻ can be, for example, the amount of Br⁻ or Br-comprising composition needed to maintain the circulating levels of Br⁻ to those concentrations that are required for eliciting the desired biological response following administration. For example, the desired biological response can be preventing cardiovascular disease in a subject and a nutritionally effective amount of Br⁻ can be the amount of Br⁻ needed to maintain the serum Br⁻ levels in the subject between 50 μM and 1 mM.

Serum Br⁻ measurements provide a convenient bodily fluid for determining the amount of Br⁻ present in a subject. Br⁻ is also found in many other places within the body, including but not limited to whole blood, urine, plasma, saliva, sweat, hair, nails, and some cells including buccal cells. The Br⁻ concentration in many of these can be diagnostically measured, such as determining urinary or plasma Br⁻ concentrations. The term “serum Br⁻” used herein refers the amount of Br⁻ that was either (1) identified from a test performed on serum or (2) identified in a non-serum sample and converted into the equivalent serum Br⁻ value using a conversion factor.

All saline solutions as described herein, including both Br-adequate embodiments of the invention, comprise an amount of salt, such as NaCl, dissolved in a biocompatible fluid, such as an aqueous fluid. Embodiments of the invention can be formulated with any biocompatible fluid. Non-limiting examples of biocompatible fluids comprise an aqueous solution or an oil-based solution. In some embodiments, compositions of the invention can be formulated with an aqueous fluid. In some embodiments, compositions of the invention can be formulated with water. In some embodiments, compositions of the invention can be formulated with water that is of pharmaceutical-grade or United Stated Pharmacopeia (U.S.P.)-grade purity.

The term “dietary item” shall include any product that undergoes at least one processing or culinary step prior to distribution and is consumed by a subject. Non-limiting examples of processing and culinary steps include mixing, cooking, baking, heating, chopping, chilling, freezing, packaging, canning, bagging, and storing. Non-limiting examples of dietary items include food products, dietary ingredients, medical foods, functional foods, beverages, dietary supplements, vitamins, minerals, and combinations thereof. Unprocessed, raw, or fresh foods, such as fresh fruits and vegetables, are not included herein within this term.

Chemically defined, the term “salt” can refer to an ionic molecule, where two or more ions of opposing charges are bound together through electrostatic interactions. Commonly, the term is used to refer to sodium chloride (NaCl), due to the widespread usage of NaCl in chemistry as well as in preparing, seasoning, and/or preserving foods. Herein, unless denoted otherwise, the term is used to refer specifically to NaCl. Herein, the term “table salt” refers to sodium chloride as a specific food ingredient, which is used for preparing, seasoning, and/or preserving foods. Sea salt falls outside the scope of this definition of “table salt.”

Herein, the term “food ingredient” can refer to any edible substance that is combined is with other edible substances, where the final combination is consumed as a food. The term “medical food” herein is defined by statute in the United States of America, Orphan Drug Act, section 5(b) (21 U.S.C. 360ee (b) (3)), which defines “medical food” as “a food which is formulated to be consumed or administered enterally under the supervision of a physician and which is intended for the specific dietary management of a disease or condition for which distinctive nutritional requirements, based on recognized scientific principles, are established by medical evaluation.”

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

When used in reference to a particular disease, the term “with” refers to patients that currently have the disease, are at risk of developing the disease, or have recently recovered from the disease. For example, the phrase “patients with cardiovascular disease” can be used herein to reference patients with ongoing cardiovascular disease or patients with a risk factor of cardiovascular disease, such as but not limited to consuming a high salt diet. This phrase can also refer to a patient who has recently recovered from a form of cardiovascular disease, such as but not limited to recovering from a heart attack. As another example, the phrase “subjects with acute kidney injury” can be used here in to reference subjects with an ongoing kidney injury or subjects with a risk factor of acute kidney injury, such as but not limited to subjects with sepsis. This phrase can also refer to a subject who has recently recovered from an acute kidney injury.

### Medical Need for the Invention

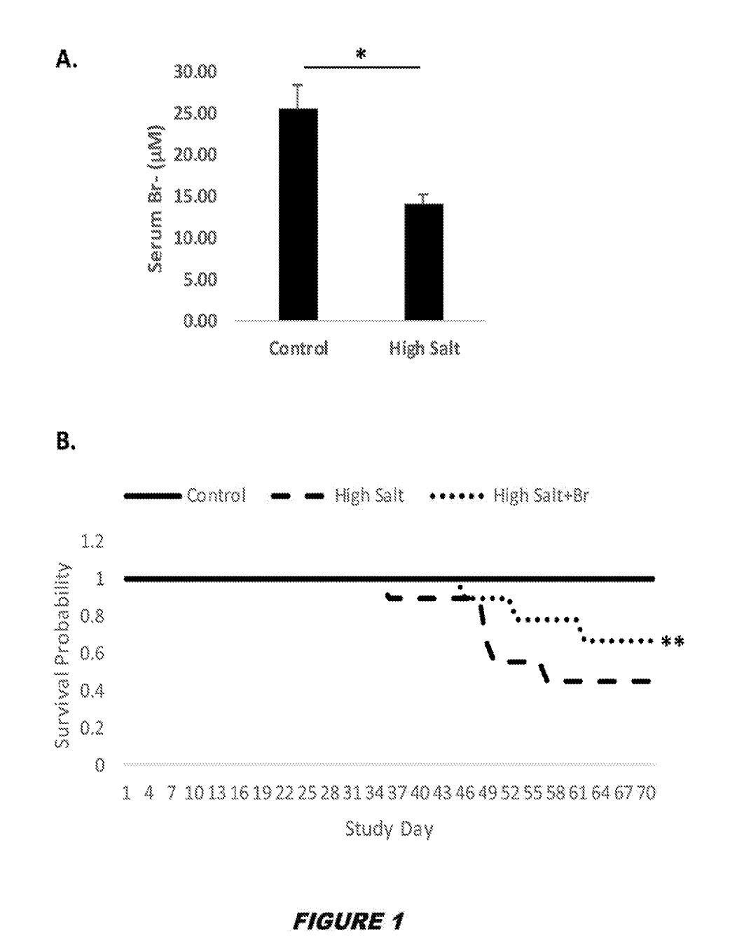

Commercially available saline solutions contain higher Cl⁻ concentrations (e.g., about 154 mM Cl⁻) than found in humans or animals (ca. about 100 mM Cl⁻ in human blood), and they also contain lower Br⁻ concentrations (e.g., about 12 μM to about 15 μM Br⁻) than found in humans or animals (ca. about 50 μM to about 100 μM Br⁻ in human plasma). As a consequence, administration of these physiologically unbalanced, Br-deficient saline solutions to a subject can simultaneously raise the in vivo Cl⁻ concentration and lower the in vivo Br⁻ concentration, causing unwanted side effects in the subject, such as the development of acidosis or hyperchloremia. As such, there is a need for improved compositions to address these unwanted side effects. Embodiments as described herein address this problem, and can eliminate or reduce these side effects, or the risk thereof, in a subject by providing compositions comprising physiologically-balanced concentrations of Br⁻ and Cl⁻. Administration of embodiments as described herein to a subject can maintain a proper in vivo concentration of Br⁻ and Cl⁻ in a subject, which is important for effectively treating a subject suffering from a disease. For example, co-administering physiologically balanced amount of Cl⁻ and Br⁻ to Dahl rats resulted in a 52% survival improvement versus rats administered a physiologically unbalanced amount of Cl⁻ alone (FIG. 1).

During development of the invention, the inventors discovered that Br⁻ ions can critically influence the course of some diseases. Thus, the inventors classify some diseases as being “Br⁻ ion dependent”. Mechanistically, Br⁻ ions can use multiple molecular pathways to influence these diseases, where non-limiting examples including acting on collagen as well as influencing natriuretic peptide signaling. In other non-limiting exmples, Br⁻ ions can influence the efficacy of treatment strategies for one of these diseases. The mechanisms used by Br⁻ and compositions and methods for practicing the invention in subjects with these diseases are described in detail herein below. In some embodiments of the invention, methods comprise treating or preventing a Br⁻ ion dependent disease in a subject. In embodiments, the method comprises administering a therapeutically effective amount of a composition comprising an amount of Br⁻ and Cl⁻ ions wherein molar amount of Br⁻ ions is between about 0.0338% and about 4.613% of the molar amount of Cl⁻. In some embodiments, the Br⁻ ion dependent disease comprises cancer, cardiovascular disease, kidney disease, end stage renal disease, an eye disease, cystic fibrosis, a degenerative disease, a blood disorder, diabetes, a wound, a skin irritation, inflammation, an injury, a medical condition requiring dialysis treatment, or a combination thereof.

### Saline Solutions of the Invention

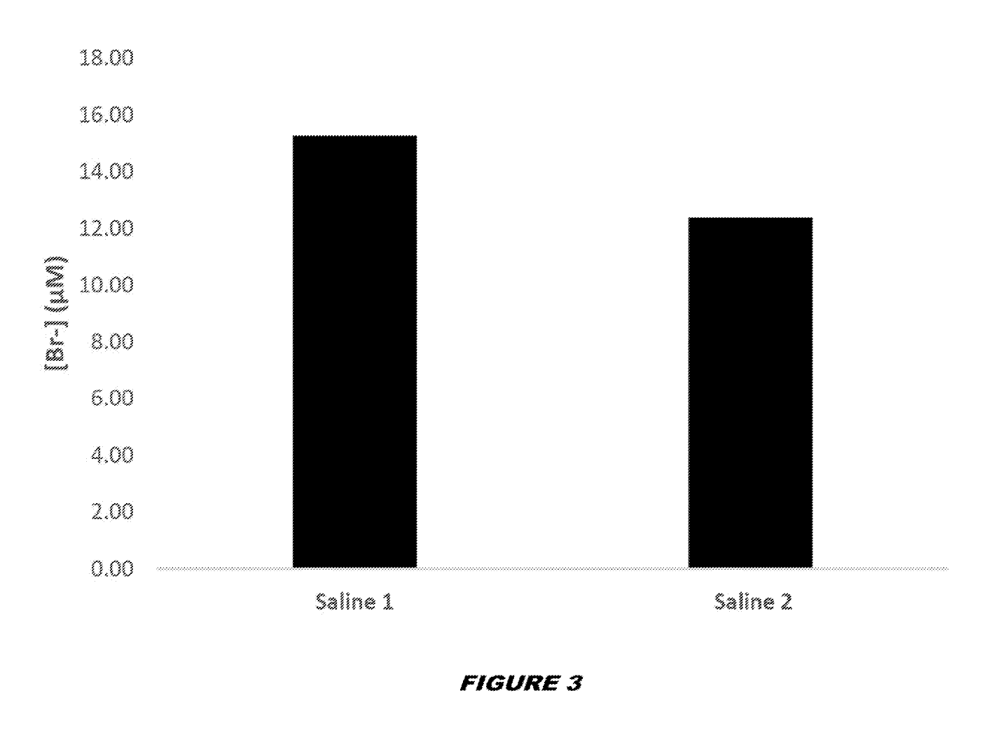

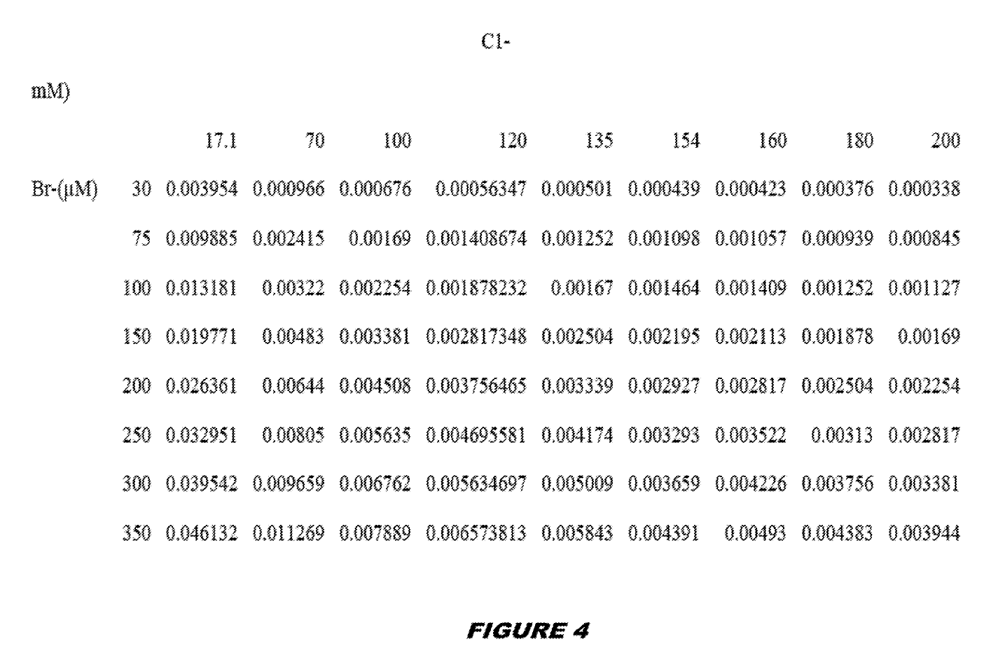

Br-deficient saline solutions are used throughout the field of medicine. Representative examples of the broad range of use for these Br-deficient saline solutions comprise saline injections, contact lens washing solutions, moisturizers for first aid bandages, and liquid vehicles for administering pharmaceutical drugs to subjects. Some Br-deficient saline solutions are regulated as drugs, such as injection saline compositions. Other Br-deficient saline solutions are regulated as medical devices, such as contact lens washing solutions. The majority of these Br-deficient saline solutions contain 0.9% NaCl. Some contain more NaCl, such as 5% or 7%, or even less NaCl such as 0.45% NaCl. In some embodiments, compositions of the invention comprise about 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 2.0%, 3.0%, 3.5%, 4.0%, 4.5%, 5.0%, 6.0%, 7.0%, 7.5%, 8.0%, 9.0%, 10.0%, 11%, 12%, 13%, 14%, 15%, 16%, 17%, 18%, 19%, 20%, or any other amount greater than about 0.1% sodium chloride. Some are supplemented with other components, including bicarbonate, potassium, lactate, and calcium. Notably, bromide ions (Br⁻) are not formulated ingredients of Br-deficient saline solutions, rather being considered contaminants of the saline solutions. For example, USP-grade Sodium Chloride is required to contain less than 100 ppm of Br⁻ (United States Pharmacopeial Convention, “Sodium Chloride” monograph, 2002), which equates to a theoretical concentration of about 15 μM Br⁻ or less in a 154 mM NaCl saline. Indeed, as described herein, Br⁻ concentration in two separate commercially-available Br-deficient saline solutions was measured, demonstrating concentrations of 15.27 μM Br⁻ and 12.39 μM Br⁻, respectively (FIG. 3). These low concentrations of Br⁻ are lower than the concentrations of Br⁻ found in healthy adults, which is between about 50 μM and about 100 μM Br⁻ in the subject's blood. Consequently, upon administration to a subject, these Br-deficient salines can distort the in vivo Br⁻ and Cl⁻ concentrations in said subject. This contrasts with embodiments of the invention, which are physiologically balanced and comprise at least about 30 μM Br⁻ in order to, upon administration to a subject, promote a healthy balance of Br⁻Cl⁻ in a subject.

Embodiments of the invention as described herein are physiologically balanced and can restore or maintain physiologically relevant levels of Br⁻, Cl⁻, or a combination thereof in vivo in a subject, such as a subject in need of saline treatment. For example, embodiments as described herein can restore or maintain physiologically relevant levels of Br⁻, Cl⁻, or a combination thereof in the blood of a subject in need of a saline treatment. Healthy adults contain about 100 mM Cl⁻ and between about 50 μM and about 100 μM Br⁻ in their blood. Physiologically, kidneys are responsible for maintaining the circulating concentrations of both halides. For example, if excess Cl⁻ or Br⁻ is consumed by a subject, the subject's kidneys will likely respond by increasing the amount of Cl⁻ or Br⁻ that is excreted in the subject's urine. Alternatively, if too little Cl⁻ or Br⁻ is consumed, the subject's kidneys will actively reabsorb greater amounts of Cl⁻ or Br⁻ and return these ions back into circulation. Thus, Cl⁻ and Br⁻ are held in balance within healthy adults. In contrast, Br-deficient 0.9% saline does not contain a physiologically balanced amount of halides, and administration of Br-deficient 0.9% saline can expose a subject to unnecessary side effects such as hyperchloremia and acidosis. The invention is designed to expose a subject to less risk than other Br-deficient saline solutions, such as 0.9% NaCl, by containing physiologically balanced amounts of Br⁻ and Cl⁻. As a result, embodiments of the invention are more similar to the biologic fluid composition of a subject, compared to Br-deficient 0.9% saline, and are thereby better suited for use within a medical setting.

Embodiments of the invention comprise physiologically balanced amounts of Br⁻ and Cl⁻ ions. For example, the Br⁻ and Cl⁻ can be within a single embodiment. For example, a physiological balanced amount of Br⁻ and Cl⁻ can be where an amount of Br⁻ is between about 30 μM and about 1 mM and an amount of Cl⁻ that is between about 17.1 mM and about 200 mM. Physiologically balanced amounts of Br⁻ and Cl⁻ can contain an amount of Br⁻ is between about 50 μM and about 500 μM and an amount of Cl⁻ that is between about 80 mM and about 120 mM. These concentrations of Br⁻ and Cl⁻ can be found in various places within an organism, such as but not limited to blood, plasma, and serum.

Circulating concentrations of Br⁻ above about 12 mM in a subject can cause the development of a toxic side effect of Br⁻ in said subject (Van Leeuwen and Sangster, 1987). Embodiments as described here are specifically formulated as to not result in toxicity in a subject. As described herein, circulating levels below about 20 μM can cause Br-deficiency (FIGS. 1,2) in a subject. The amount of Br⁻ within a composition of the invention described herein is sufficient to prevent or treat Br-deficiency in a subject, but is also less than that amount of Br⁻ required to elevate the circulating levels of Br⁻ to above about 1 mM in a subject. In some embodiments, compositions can contain just enough Br⁻ to raise or maintain the circulating amount of Br⁻ in a subject at about 30 μM. Some embodiments contain just enough Br⁻ to raise or maintain the circulating amount of Br⁻ in a subject at about 50 μM. In embodiments, compositions of the invention described herein contain just enough Br⁻ to raise or maintain the circulating amount of Br⁻ in a subject at about 100 μM. Some embodiments contain just enough Br⁻ to raise or maintain the circulating amount of Br⁻ in a subject at about 150 μM. In embodiments, compositions of the invention described herein contain just enough Br⁻ to raise or maintain the circulating amount of Br⁻ in a subject at about 200 μM. Some embodiments contain just enough Br⁻ to raise or maintain the circulating amount of Br⁻ in a subject at about 250 μM. Some embodiments contain just enough Br⁻ to raise or maintain the circulating amount of Br⁻ in a subject at about 300 μM. In embodiments, compositions of the invention described herein contain just enough Br⁻ to raise or maintain the circulating amount of Br⁻ in a subject at about 400 μM. Some embodiments contain just enough Br⁻ to raise or maintain the circulating amount of Br⁻ in a subject at about 500 μM. In embodiments, compositions of the invention described herein contain just enough Br⁻ to raise or maintain the circulating amount of Br⁻ in a subject at about 600 μM. Some embodiments contain just enough Br⁻ to raise or maintain the circulating amount of Br⁻ in a subject at about 700 μM. In other embodiments, compositions contain just enough Br⁻ to raise or maintain the circulating amount of Br⁻ in a subject at about 800 μM. Some embodiments contain just enough Br⁻ to raise or maintain the circulating amount of Br⁻ in a subject at about 900 μM. Some embodiments contain just enough Br⁻ to raise or maintain the circulating amount of Br⁻ in a subject to an amount between about 30 μM and about 1 mM. Some embodiments contain just enough Cl⁻ to raise or maintain the circulating amount of Cl⁻ in a subject at about 100 mM.

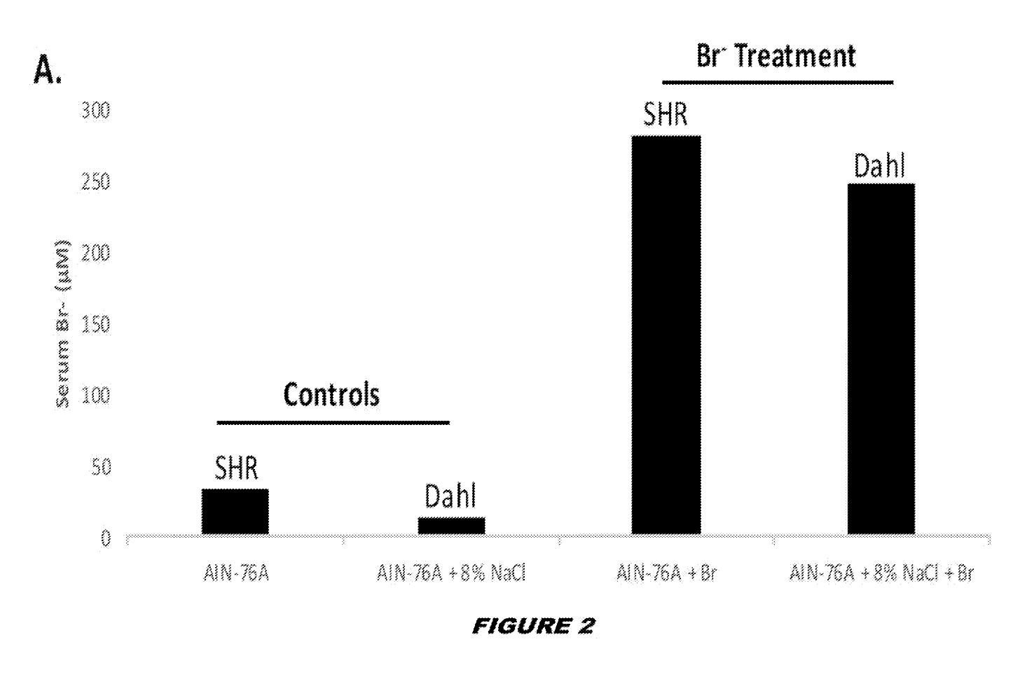

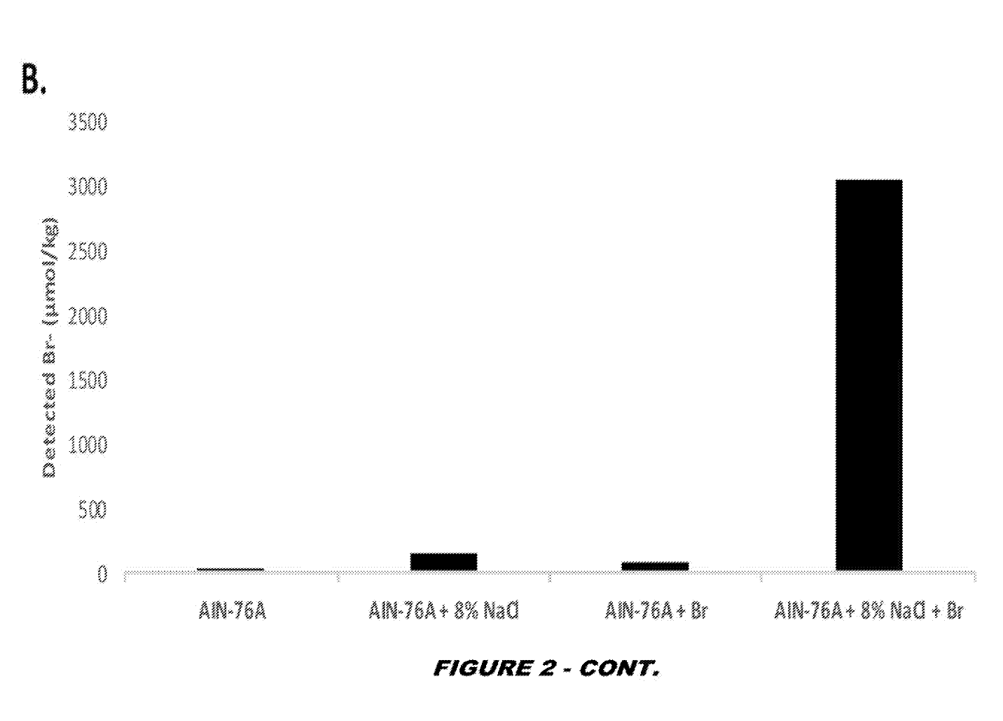

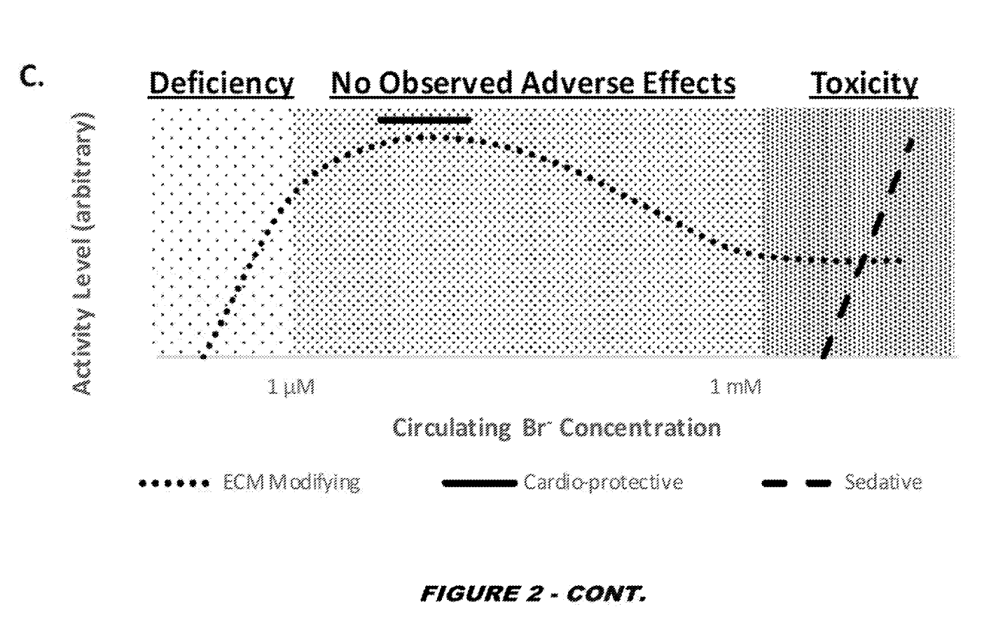

Various circumstances can disturb the balance of Br⁻ and Cl⁻ in a subject. For example, dialysis treatments, utilizing Br-deficient salines or Br-deficient dialysates, can remove Br⁻ from a subject, resulting in Br⁻ deficiency. For example, levels of about 12 μM serum Br⁻, which is less than the physiologically normal amount of between about 50 μM and about 100 μM of Br⁻, have been reported in some subjects post-dialysis (Miura et al., 2002). Without being bound by theory, at this level of Br⁻ and assuming the Cl⁻ level is approximately 100 mM in blood, a subject would possess an imbalanced amount of Br⁻ and Cl⁻. As another example, certain drugs can remove Br⁻ from a subject. Non-limiting examples of such drugs comprise diuretics and cancer chemotherapies. As yet another example, certain dietary habits of a subject can deplete the subject of Br⁻. Non-limiting examples of such dietary habit comprise high salt diets, high sodium diets, and lack of fresh vegetables or seafood or nuts. In all these examples, Br⁻ can be depleted to a level wherein the subject displays an imbalance of Br⁻ and Cl⁻. Embodiments of the invention can be used to correct or restore an imbalanced amount of Br⁻ and Cl⁻ in a subject to physiologically balanced levels, and at the same time not result in toxicity. For example, daily administration of a physiologically balanced amount of Br⁻ and Cl⁻ to rats over the course of 10 weeks resulted in serum Br⁻ levels that are well below those levels required to induce bromide-associated toxicity (FIG. 2), evidencing the safety of embodiments of the invention.

Br-deficient saline solutions are physiologically unbalanced, and poorly reflect the electrolyte composition of human serum or blood. Br-deficient 0.9% saline contains approximately 154 mM of Cl⁻ ions, which is a concentration about 50% higher than the 100 mM Cl⁻ that is typically found in serum. As a result, the administration of Br-deficient 0.9% saline to subjects can induce side effects due to the elevated concentration of Cl⁻ in the saline relative to the subject's serum. For example, subjects with sepsis or acute kidney injury are frequently administered intravenous Br-deficient saline. During such treatment, these subjects must be monitored for development of hyperchloremia and acidosis as a result of Br-deficient saline administration. As another example, administration of too much Br-deficient 0.9% NaCl to a subject with diabetes can cause the onset of metabolic acidosis in said subject.

The potential for side effects following Br-deficient saline administration represents an inherent defect of Br-deficient saline formulations. By not providing a physiologically-balanced composition, the Br-deficient saline can alter the physiologic salt balance within the subject. Considering that the subjects who receive these Br-deficient saline solutions are already trying to recover or heal from another injury or disease, which the Br-deficient saline is intended to treat, it is indeed counter-productive for a subject to encounter a saline-induced side effect as a result of treatment with a physiologically unbalanced Br-deficient solution. The experiencing of such side effects can compound or worsen the condition of the subject who is already battling the primary injury or disease, and can lengthen the road to recovery. Embodiments of the invention can overcome the unwanted side effects associated with administration of Br-deficient saline solution, by offering a physiologically-balanced Br-containing composition for administration to a subject.

Tonicity is an important property of medical solutions, and saline solutions can indeed provide an osmotically balanced fluid for treating a subject. For example, Br-deficient 0.9% NaCl is isotonic. However, this formulation contains a higher concentration of salt than found in the human body, which can be detrimental for subjects. In other words, Br-deficient 0.9% NaCl achieves its isotonicity through a non-physiologic concentration of NaCl. As mentioned above, Br-deficient 0.9% saline has an approximately 50% higher concentration of Cl⁻ than is found in blood. Upon administering Br-deficient 0.9% saline to a subject, this can effectively disturb the local or systemic halide concentrations which can trigger side effects in a subject. For example, administration of Br-deficient 0.9% saline to subjects with sepsis or kidney disease can cause the subject to develop hyperchloremia and/or acidosis. Embodiments of the invention can be isotonic, hypertonic, or hypotonic while comprising a physiologically balanced amount of Br⁻ and Cl⁻.

By formulating a physiologically balanced amount of Br⁻ and Cl⁻ into the saline, embodiments as described herein can minimize the risk of treatment complications encountered by a subject who requires a saline treatment. As shown in the data presented herein, Br⁻ was used to reduce mortality events associated with administering high amounts of NaCl to Dahl rats (FIG. 1). While rats on a high salt (8% NaCl) diet experienced approximately 44% survival probability, rats fed a high salt diet with supplemental Br⁻ demonstrated a 67% survival probably. Thus, the administration of Br⁻ and Cl⁻ together improved survival probability by approximately 52%.

As a potential mechanism for understanding the importance of Br⁻, excess amounts of Cl⁻ can induce the accelerated depletion of Br⁻ from a subject. Thus, upon administration to a subject, Cl-based saline solutions that lack Br⁻, such as Br-deficient 0.9% NaCl saline, can render a subject Br-deficient. This can worsen the halide balance in a subject, first by administered supra-physiologic amounts of Cl⁻ to the subject and secondly by depleting Br⁻ from said subject. As describe herein, embodiments of the invention can overcome this halide imbalance by administering physiologically balanced amounts of Br⁻ and Cl⁻ to a subject.

Owing to the vastly greater concentration of Cl⁻ over Br⁻ in human serum, more clinical and research attention has been devoted to Cl⁻ while Br⁻ has been ignored and misunderstood. As a consequence, Br⁻ has not be formulated into current saline products. Through data presented herein, it is now shown that maintaining proper Br⁻ concentration in vivo is important for the health of a subject. The invention provides physicians and other consumers with an improved saline product that more accurately mimics the halide composition of bodily fluids in healthy adults. Without being bound by theory, the invention can improve outcomes in a subject as a result of improved physiological balance when compared to all other physiologically unbalanced saline solutions that are commercially available.

Br-deficient medical saline solutions were developed in the 1800's by physicians such as William Brooke O'Shaughnessy, Thomas Latta, Sydney Ringer, and Hartog Jacob Hamburger. When compared to the practice of medicine without any type of saline, indeed, even basic saline solutions have provided a substantial benefit to medicine. Yet deficiencies in the science of saline solutions have persisted, such as the physiologically imbalanced concentrations of Br⁻ and Cl⁻. Such deficiencies can result in saline-induced side effects in subjects, such as hyperchloremia and acidosis. Therefore, while the current formulations of Cl-based saline solutions is indeed a notable medical advancement, data presented herein (FIGS. 1,2) further advances the science of saline solutions by teaching the importance of administering physiologically balance halide solutions to a subject. Furthermore, embodiments of the invention are chemically distinguished by comprising physiologically-relevant amounts of Br⁻ and Cl⁻ for administration to a subject, which upon administration to a subject, can prevent the development of unwanted saline-induced side effects.

The lack of a perceived physiologic need for Br⁻ is reflected in the physiological unbalance of the Br-deficient saline products currently being used. Moreover, there has persisted a misinformed notion that Br⁻ is toxic to subjects. Collectively, Br⁻ has been ignored as a formulated ingredient of Br-deficient saline solutions. In contrast, embodiments as described herein formulated to administer safe amounts of both Br⁻ and Cl⁻ to a subject in need thereof. Such formulations as found in embodiments of the invention, disclosed herein, can be used as a physiologically balanced halide solution that is administered or applied to a subject or a medical device.

Embodiments of the invention can use many different means to detect or measure Br⁻ and/or Cl⁻ in a fluid or tissue, such as that found in a subject. In embodiments, levels of Br⁻ can be measured in bodily tissues or fluids. Non-limiting examples of such tissues and fluids comprise whole blood, serum, plasma, saliva, urine, sweat, hair, cellular samples such as buccal cells, or nails. Non-limiting examples of technologies for detecting or measuring Br⁻ and/or Cl⁻ in a subject include column chromatography, inductively coupled plasma mass spectrometry, elemental x-ray analysis, x-ray florescence, and neutron activation analysis. Such tools can be utilized in embodiments of the invention. Each technique offers various advantages and disadvantages, such as cost, detection limits, and the amount of sample required. For example, mass spectrometry and column chromatography can be used in methods as described herein for quantifying the levels of bromide from subject samples, including blood and urine samples. Some embodiments of the invention utilize inductively coupled mass spectrometry for measuring Br³¹ . The element bromine is found as two isotopes with nearly equal proportion in nature, Br⁷⁹ and Br⁸¹, which assists in identifying Br⁻ by mass spectrometry. Mass spectrometry also offers a lower detection limit than column chromatography, allowing a clinician to more accurately measure Br⁻ in a subject who may be deficient in Br³¹ .

Chromatographic clinical tests for measuring Br⁻ in subject samples were developed in response to the widespread yet mistaken belief that Br⁻ is inherently unsafe in humans. Consequently, many of these tests, and their accompanying methods, are designed to detect Br⁻ toxicity rather than Br-deficiency. Nonetheless, the existence of these methods better enables the practice of the invention by providing readily-available diagnostic methods for measuring Br⁻ levels in subject samples. As stated above, mass spectrometry provides suitable sensitivity for detecting low levels of Br⁻ in a sample from a subject.

In addition to halide imbalance induced by administering a Br-deficient saline to a subject, halide imbalance can also result from administration of excessive amounts of Br⁻ to a subject leading to toxicity. Historically, large doses of bromide, such as approximately 0.8-1.6 g Br⁻ administered every 3-4 hours, were previously used as an early pharmaceutical sedative, anti-libido, and anti-epileptic treatment. Early references, such as the 1920 Handbook of Pharmacy and Therapeutics by Eli Lilly and Co., describe such products comprising high concentrations of bromine. These products relied on the ability of high concentrations of Br⁻ to suppress neural activity. These high physiological concentrations of Br⁻ can result in unwanted side effects, including chronic toxicity.

Over time, the historical sedative usage of Br⁻ was discontinued due the combination of (1) the risk of developing bromism and/or bromoderma and (2) the development of barbiturates and other modern sedatives. Van Leeuwen and Sanger (1987) published a comprehensive review of Br⁻ toxicity, where the medical usage of Br⁻ had been largely discontinued by publication of this review. Unfortunately, the historical usage of Br⁻ salts has mistakenly branded Br⁻ as a crude sedative treatment.

The anti-epilepsy effect of Br⁻ occurs when circulating Br⁻ levels are above approximately 6 mM. In dogs, for example, the reported therapeutic range of serum Br⁻ levels that are targeted for eliciting a sedative effect is between 12.5 mM and 37.5 mM for subjects beginning treatment with Br⁻ alone, above 25 mM for dogs that do not response to an initial course of Br⁻ treatment, and between 12.5 mM-31 mM when Br⁻ is used in addition to a phenobarbital (Trepanier et al., 1998; Podell et al., 1993). As another example, in humans the targeted therapeutic range to elicit a sedative effect is reported to be between 6 mM-12 mM plasma Br⁻ (Van Leeuwen & Sangster, 1987). These elevated concentrations of Br⁻ represent another form of imbalanced halide levels.

On the other hand, embodiments as described herein comprise saline solutions with physiologically balanced amounts of Cl⁻ and Br⁻. For example, embodiments as described herein seek to restore or maintain Br⁻ levels in a subject between about 30 μM and about 100 mM. Thus, rather than pharmacologically elevating either Cl⁻ or Br⁻ to supra-physiological levels, such as approximately 6 mM plasma Br⁻ achieved through previous use of Br⁻ as a sedative drug, embodiments of the invention can be used to achieve and maintain a proper physiologic halide balance in a subject. For example, the highest serum Br⁻ concentration achieved through practice of the invention (FIG. 2) was about 40-fold lower than the excessive Br⁻ concentrations achieved during the previous administration of Br⁻ as a sedative treatment. Some embodiments comprise only those amounts of Br⁻ required to maintain a subject's serum Br⁻ between about 30 μM and about 1 mM.

Physiologically balanced amounts of Cl⁻ and Br⁻, as described in embodiments herein, synergistically achieve better outcomes than administering to a subject an amount of Br⁻ alone or an amount of Cl⁻ alone. In other words, embodiments of the invention provide outcomes that can only be realized through the co-administration of physiologically balanced amounts of Br⁻ and Cl⁻ together. If administered by itself to a subject, too much Cl⁻ or too much NaCl can result in health risks such as hyperchloremia or metabolic acidosis, for example. If administered by itself to a subject, high doses of Br⁻ can result in unwanted side effects, such as bromide-induced toxicity as described herein. However, if amounts of Br⁻ as described herein are co-administered to a subject together with amounts of Cl⁻ as described herein, the Br⁻ can reduce or eliminate the health risks due to administration of too much Cl⁻ or too much NaCl (see FIG. 1), whereas the Cl⁻ prevents the accumulation of Br⁻ within the subject by enhancing the urinary excretion of Br⁻. Taken together, administering to a subject physiologically balanced amounts of Br⁻ and Cl⁻ together as described herein acts synergistically to maintain physiologically balanced amounts of Br⁻ and Cl⁻ in the circulation of a subject, resulting in increased human health and reduced unwanted side effects. The importance of administrating physiologically balanced amounts of Br⁻ and Cl⁻ is highlighted by the survival benefit seen in rats administered both halides as compared to rats treated with Cl⁻ alone (FIG. 1).

The synergistic activity of embodiments of the invention can only be realized by administration of physiologically balanced amounts Br⁻ and Cl⁻ together. Physiologically balanced amounts of Br⁻ and Cl⁻ are described herein. Without being bound by theory, no other combination of halides can achieve this same effect, since no other biologically relevant halide, such as iodide or fluoride, can perform the biologic activity of Br⁻.

The following two non-limiting examples highlight the risks incurred through administration of just Br⁻ or just Cl⁻ to a subject. For example, if an amount of Br⁻ is administered without Cl⁻ to a subject, said subject would likely require monitoring to ensure that circulating levels of Br⁻ do not rise to undesired levels, such as above 1 mM in serum, at which level the subject would be at risk of bromide-induced toxicity. Conversely, and as another example, if an amount of Cl⁻ is administered without Br⁻ to a subject, such as administering 0.9% NaCl to a subject, the electrolyte levels in said subject would likely require monitoring to guard against the development of a side effect of dysregulated electrolytes. The circumstances, and specifically the treatment-associated risks, described in these two non-limiting examples can be avoided through practice of the invention. In summary, by combining physiologically balanced amounts of Br⁻ and Cl⁻, embodiments of the invention use Br⁻ to control the potential side effects of excessive Cl⁻ administration while Cl⁻ is used to regulate the final circulating Br⁻ levels that result from administration of an embodiment to a subject.

### Nutritionally Effective Amounts of Br⁻

Biologically, the causal events of disease can occur long before the symptoms of disease are clinically manifested. Deterioration of cellular or tissue health may in some cases be detected through altered levels of specific biomarkers. For example, creatinine levels can be used to monitor the state of kidney health in a subject, where the amount of creatinine is used to calculate the estimated glomerular filtration rate (eGFR) as an indicator of kidney function. Yet fundamentally, diagnostic indicators such as eGFR simply point to the outcome of an established disease etiology. In other words, by the time a symptom of disease is clinically apparent, biologic damage has already occurred.

While therapeutic drugs can be used to treat specific diseases, nutrition and dietary supplements can be used to maintain healthy levels of specific disease markers. For example, where a drug might be used to lower serum triglyceride levels from elevated levels to healthy levels and thereby reduce the subject's risk of heart disease, a healthy diet might be used to maintain healthy serum triglyceride levels as a means of promoting heart health.

It is known that poor dietary habits can have significant negative impacts on the health of the heart, kidneys, and vasculature. For example, a high sodium diet can elevate one's risk of developing hypertension. Consequently, patients are frequently advised by physicians to adopt healthy dietary habits, yet prevalence rates are persistently elevated.

The element bromine is a trace element in the bloodstream of humans and animals, in the form of bromide (Br⁻). Certain subjects who are at risk of developing heart, kidney, and vascular diseases as disclosed herein, are chronically deficient in Br⁻. Non-limiting examples of subjects that can be chronically deficient in Br⁻ comprise subjects who consume a high salt or high sodium diet, subjects receiving dialysis therapy, subjects who take diuretic agents, and subjects who are receiving chemotherapy treatments for cancer. In some cases, the depletion of Br⁻ can occur as the result of a prior medical treatment. For example, diuretic and chemotherapeutic drugs can be used to treat hypertension and cancer in a subject, respectively, yet both can also reduce the amount of Br⁻ in circulation. Based on data presented herein, the inventors point out that this loss of Br⁻ can damage the health of the hearts and kidneys in these subjects.

Embodiments of the invention can improve the heart health, kidney health, and/or vascular health in subjects, such as patients on maintenance dialysis and others, by administering a nutritionally effective amount of Br⁻ to the subject. In the case of dialysis patients, dietary supplements are important for maintaining proper nutritional status in the patient. Some supplements are even formulated and marketed specifically for dialysis patients. However, these specialty supplements for dialysis patients are not formulated to contain nutritionally effective amounts of Br⁻. Unfortunately, these patients are at high risk of developing diseases, such as cardiovascular disease and blood disorders. The invention provides compositions and methods for administering a nutritionally effective amount of Br⁻ to a subject, such as those undergoing dialysis.

The health of heart, kidneys, and vasculature can often be monitored through routine measurements of certain serum, blood, or urinary biomarkers. Non-limiting examples of these biomarkers include lipids, cholesterol, high-density lipoprotein, low-density lipoprotein, very-low-density lipoprotein, homocysteine, creatinine, c-reactive protein, cystatin C, tryglyceride, protein, albumin, lipid oxidation, protein oxidation, kidney injury molecule 1, neutrophil gelatinase-associated lipocalin, protein, albumin, systolic blood pressure, diastolic blood pressure, heart rate, peptides of collagen IV or III, brain natriuretic peptide (BNP), atrial natriuretic peptide, pro-BNP, cyclic guanosine monophosphate, and glomerular filtration rate. When using these biomarkers to monitor health, the subject undergoes a specific test to measure the biomarker level in serum, blood, or urine, and the test results are compared to a reference range of results in order to determine whether the results indicate that the subject is healthy. For example, the kidney health of subjects are often assessed through regular testing of creatinine and albumin levels. The results from these tests are used to calculate the estimated glomerular filtration rate (eGFR) and albumin-creatinine ratio (ACR) in order to assess whether the subject's kidneys are functioning properly. In healthy adults, an eGFR value above 60 ml/min/1.73 m² and an ACR value below 3 mg/mmol indicates a healthy kidney (NICE Guideline, 2014). Obviously, physicians and subjects alike are highly motivated to see biomarkers remain within healthy levels over long periods of time, indicating that the subject's heart, kidney, and vasculature are remaining in good health. Embodiments of the invention can be used to assist in maintaining biomarkers of heart, kidney, and/or vascular health within a healthy range.

Dietary supplements are, in part, distinguished from drugs in that supplements can be used to promote health while drugs are used to prevent, treat, or diagnose disease. For example, where a supplement may assist a subject in maintaining a healthy eGFR, a drug may be administered as a pharmaceutical strategy for raising the eGFR in a subject whose eGFR is too low. Some embodiments of the invention may be classified as drugs for treating or preventing heart disease, kidney disease, and/or vascular disease which act by administering a nutritionally effective amount of Br⁻ to a subject.

Br⁻ is popularly misunderstood to be unhealthy or even toxic. Such conventional beliefs have impeded the discovery of any role for Br⁻ in heart, kidney, and/or vasculature. Without being bound by theory, the invention provides compositions and methods that use nutritionally effective amounts of Br⁻ to promote the health of a subject, such as heart health, kidney health, and vasculature health.

In contrast to the popular belief that Br⁻ is toxic, through data presented herein, the inventors show that a deficiency of Br⁻ can cause disease in animals. Based on data presented herein (FIG. 1), the inventors here disclose that Br-deficiency can occur when serum Br⁻ levels are at or below 20 μM. Serum Br⁻ levels above 12 mM place the patient at risk of developing a toxic side effect of Br³¹ . Thus, in general, disease symptoms caused by too much or too little Br⁻ should not appear when serum Br⁻ levels are between about 20 μM and about 12 mM, in the absence of other confounding factors such as elevated levels of thiocyanate or high dietary salt intake. Healthy adults contain on average of 67 μM plasma Br⁻ (Van Leeuwen & Sangster, 1987). For the purpose of practice of the invention, the inventors hereby disclose that the target range of serum Br⁻ in a healthy adult should be between about 50 μM and about 1 mM. In order to prevent Br-deficiency, the invention may be prescribed or administered to a patient whose serum Br⁻ levels are at or below 50 μM.

Embodiments of the invention can use many different means to detect or measure Br⁻ in a subject. In embodiments, Br⁻ can be detected and levels of Br⁻ can be measured in bodily tissues or fluids. Non-limiting examples of such tissues and fluids include whole blood, serum, plasma, saliva, urine, sweat, hair, or nails. Non-limiting examples of means to detect or measure Br⁻ in a subject include column chromatography, inductively coupled plasma mass spectrometry, x-ray florescence, and neutron activation analysis. Such tools can be utilized in embodiments of the invention. Each technique offers various advantages and disadvantages, such as cost, detection limits, and the amount of sample required. For example, mass spectrometry and column chromatography can be used in methods as described herein for quantifying the levels of bromide from patient samples, including blood and urine samples. Some embodiments of the invention utilize inductively coupled mass spectrometry for measuring urinary Br⁻ as well as column chromatography to measure Br⁻ in blood. Mass spectrometry offers a lower detection limit than column chromatography, allowing a clinician to more accurately measure Br⁻ in a patient who may be deficient in Br⁻.

Chromatographic clinical tests for measuring Br⁻ in patient samples were developed in response to the widespread yet mistaken belief that Br⁻ is inherently unsafe in humans. Consequently, many of these tests, and their accompanying methods, are designed to detect Br toxicity rather than Br-deficiency. Nonetheless, the existence of these methods better enables the practice of the invention by providing readily-available diagnostic methods for measuring Br⁻ levels in patient samples. As stated above, mass spectrometry provides suitable sensitivity for detecting low levels of Br⁻ in patient samples.

These analytical methods are also suitable for measuring the amount of Br⁻ in a dietary item. Non-limiting examples of dietary items include food products, dietary ingredients, medical foods, functional foods, beverages, dietary supplements, vitamins, minerals, and combinations thereof. Non-limiting examples of analytical methods useful for measuring the amount of Br⁻ in a dietary item include column chromatography, inductively coupled plasma mass spectrometry, x-ray florescence, and neutron activation analysis. For example, the amount of Br⁻ in a sample of a medical food can be measured before the medical food is administered to a subject in need thereof. Some embodiments of the invention comprise supplementing a dietary item with an amount of Br⁻, such as an amount between 1 and 2 mg of Br⁻. In embodiments, the amount of Br⁻ in the dietary item comprises only enough Br⁻ to raise circulating levels of Br⁻ to about 20 μM.

Embodiments of the invention comprise ionic forms of Br⁻, such as sodium bromide (NaBr), potassium bromide (KBr), or magnesium bromide (MgBr₂). This contrasts with other various other examples of the element bromine in commercial products such as methyl bromide (CH₃Br), a soil fumigant, and brominated vegetable oils, used in drinks such as Mountain Dew®, which all use non-ionic forms of the element.

An amount of Br⁻ present in food is a contamination (Van Leeuwen and Sangster, 1987), as Br⁻ is not intentionally added to foods. On the other hand, a distinguishing feature of embodiments of the invention is the specific incorporation of an amount between about 0.1 mg and 25 mg of Br⁻ into a serving of a dietary item. In embodiments, the amount of Br⁻ in the dietary item comprises only enough Br⁻ to raise circulating levels of Br⁻ to about 20 μM. This Br⁻ may be added to traditional foods in order to enrich or supplement the food with a nutritionally effective amount of Br⁻.

In the United States, dietary supplements, vitamins, minerals, and drugs are required to be manufactured in accordance with current good manufacturing principles (cGMP). Thus, for some embodiments, the invention can comprise a dietary supplement that is manufactured under cGMP conditions; contains a nutritionally effective amount of Br⁻, and promotes the health of a subject, such as heart health, kidney health, and/or vascular health.

The inventions described herein use the activity of Br⁻ to promote heart health, kidney health, and/or vascular health in a subject. Accordingly, embodiments of the invention rely on circulating Br⁻ levels and Br-comprising compositions to evaluate and promote the health of these organs in a subject. The inclusion of other components, such as taurine, is not necessary for embodiments of the invention to perform their activity. In embodiments of the invention, less than 1 g of taurine can be administered to a subject. In some embodiments, undetectable amounts of taurine can be administered to a subject. In other embodiments of the invention, the compositions described herein do not contain taurine.

There are numerous methods for preparing and packaging food products, beverages, and drinks. Non-limiting examples include fresh, frozen, prepared, refrigerated, canned, boxed, bottled, and flavored. These methods are all compatible with preparing and packaging embodiments of the invention. A distinguishing feature of the invention is adding a nutritionally effective amount of Br⁻ to the products that are generated through these methods.

It has been shown, in Drosophila as well as goats, that Br-deficiency can cause lethality in developing animals (McCall et al., 2014; Haenlein and Anke, 2011). Interestingly, adult flies and goats displayed little to no phenotype, indicating that bromide is required during tissue development but may be dispensable or have little activity upon reaching adulthood (e.g. when tissue development slows or stops). Heart, kidney, and/or vascular phenotypes were not observed, and a putative threshold of Br-deficiency was not established in these studies. Finally, there has never been evidence that Br⁻ can be used to promote heart health, kidney health, and/or vascular health in a Br-deficient animal. Thus, a distinguishing feature of the invention is the administration of a nutritionally effective amount of Br⁻ to promote heart, kidney, and/or vascular health in adults.

### Historical Use of Br⁻ in Medicine

Large doses of bromide, such as approximately 0.8-1.6 g Br⁻ administered every 3-4 hours, were previously used as an early pharmaceutical sedative, anti-libido, and anti-epileptic treatment. Historical references, such as the 1920 Handbook of Pharmacy and Therapeutics by Eli Lilly and Co., describe such products comprising high concentrations of bromine. Unlike embodiments of the invention, these products relied on the ability of high concentrations of Br⁻ to suppress neural activity. As described herein, high physiological concentrations of Br⁻ can result in unwanted side effects, including chronic toxicity. Importantly, these sedative and toxic Br⁻ concentrations are more than 40-fold higher than the highest demonstrated in vivo Br⁻ level achieved through practicing embodiments of the invention. Furthermore, the invention is neither designed to treat neurologic symptoms nor does it act through neurologic-based mechanisms. The invention was developed as a nutritional strategy for improving heart health, kidney health, and/or vascular health in a subject. Importantly, this nutritional effect occurs below those therapeutic levels where Br⁻ can modulate neurologic disease in a subject.

The ability of Br⁻ to effectively modulate neural activity is only known to occur when the systemic concentrations of Br⁻ are greatly elevated above their normal concentrations. Furthermore, the high target concentrations for achieving a sedative effect are close to those concentrations where Br⁻ causes unwanted side effects in patients. Indeed, when used for these early sedative purposes, therapeutic concentrations were often 6 mM and higher, while side effects can occur over 12 mM (van Leeuwen and Sangster, 1987). Chronic toxicity occurs above this higher threshold, manifesting as a set of neurologic and/or dermatologic symptoms termed bromism and bromoderma, respectively. With respect to the invention, these elevated levels of Br⁻ are far beyond (1) the normal Br⁻ concentrations seen in healthy adults; (2) the Br⁻ concentrations found in the compositions and methods disclosed here; and (3) the final Br⁻ concentrations to be circulating in subjects after receiving a nutritionally effective amount of Br⁻, such as during practice of the invention. For example, embodiments of the invention, when practiced on a patient receiving maintenance dialysis, may be designed to raise the serum Br⁻ concentration to between 50 μM-1 mM, which is well below the threshold for modulating neural activity.

Historically, administering high doses of bromide was indeed an effective sedative treatment. Over time, however, this medical usage was discontinued due the combination of (1) the risk of developing bromism and/or bromoderma and (2) the development of barbiturates and other modern sedatives. Van Leeuwen and Sanger (1987) published a comprehensive review of Br⁻ toxicity, where the medical usage of Br⁻ had been largely discontinued by publication of this review. Unfortunately, the historical usage of Br⁻ salts has mistakenly branded Br⁻ as a crude sedative treatment.

While Br⁻ has historically served as an effective sedative, albeit with elevated risk of toxicity, such historical usage qualifies as palliative, non-curative care, which is quite different than utilizing nutritional strategies for maintaining health. Indeed, embodiments of the invention are designed to administer nutritionally effective amounts of Br⁻ to subjects for the purpose of improving heart health, kidney health, and/or vascular health.

Currently, KBr is only used therapeutically as a sedative for refractory pediatric cases of seizures and in canine epilepsy (Baird-Heinz, 2012). Additionally, bromide ions are used as counter ions in compositions for anesthesiology or pain indications, where the mechanism of action involves modulating a neurotransmitter receptor. Importantly, none of these drugs provide nutrition to a subject. Certain homeopathic drugs also contain bromide for psychological and/or neurologic disorders.

The anti-epilepsy effect of Br⁻ occurs when circulating Br⁻ levels are above approximately 6 mM. In dogs, for example, the reported therapeutic range of serum Br⁻ levels that are targeted for eliciting a sedative effect is between 12.5 mM and 37.5 mM for subjects beginning treatment with Br⁻ alone, above 25 mM for dogs that do not respond to an initial course of Br⁻ treatment, and between 12.5 mM-31 mM when Br⁻ is used in addition to a phenobarbital (Trepanier et al., 1998; Podell et al., 1993). As another example, in humans the targeted therapeutic range to elicit a sedative effect is reported to be between 6 mM-12 mM plasma Br⁻ (Van Leeuwen & Sangster, 1987).

On the other hand, embodiments as described herein comprise administration of nutritionally effective amounts of Br⁻ and Br-comprising compositions to promote and maintain the health of the heart, kidneys, and/or vasculature. Moreover, embodiments of the invention are designed to assist in maintaining serum Br⁻ levels between about 50 μM and 1 mM in a subject, when the invention is practiced as taught herein. This stands in sharp contrast to the high administered doses of Br⁻ and the elevated therapeutic range (e.g. >6 mM) targeted in the use of Br⁻ as a sedative drug to treat neurological and psychological diseases. For example, the highest serum Br⁻ concentration achieved through practice of the invention (FIG. 2) was 40-fold lower than the excessive Br⁻ concentrations achieved during the previous sedative and toxic administration of Br⁻. In embodiments, the disease is not a neurological disease. In embodiments, the disease is not a psychological disease. In some embodiments, nutritionally effective amounts of Br⁻ comprise low doses of Br³¹ . In embodiments, only an amount of Br⁻ or Br-comprising composition sufficient to maintain the circulating levels of Br⁻ above 50 μM is administered to the subject. In other embodiments, the invention comprises compositions comprising an amount of Br⁻ sufficient to maintain the serum levels of Br⁻ to an amount between about 50 μM and 1 mM.

Embodiments of the invention are designed to administer a nutritionally effective amount of Br⁻ that is sufficient to accomplish the desired activity, while at the same time prevent the onset of unwanted side effects and toxicity associated with the overdosing of Br⁻ to the subject. In humans, Br⁻ toxicity can develop when serum Br⁻ levels approach 12 mM or higher while healthy adults typically display about 67 μM plasma Br⁻ levels (Van Leeuwen and Sangster, 1987). Critically, there has not been any understanding of the threshold, with respect to the levels of Br⁻ in a body fluid, at which the symptoms of Br-deficiency may appear. Moreover, the inventor has shown herein (FIG. 2) that the symptoms of Br-deficiency can develop when serum Br⁻ levels are below 20 μM. Thus, a serum Br⁻ concentration of 20 μM appears to approximate an important threshold for inducing Br-deficiency, where subjects who experience serum Br⁻ levels at or below 20 μM may be at risk of developing a symptom of Br-deficiency. These low levels of Br⁻ may be achieved in subjects with certain diseases or diets, such as but limited to individuals receiving dialysis treatments or individuals who regularly consume a high salt diet. For such subjects who are Br-deficient or at risk of becoming Br-deficient, embodiments of the invention are useful for the administration of nutritionally effective amounts of Br⁻ to either correct the deficiency or reduce the subject's risk of developing Br-deficiency.

In embodiments, only an amount of Br⁻ or Br-comprising composition sufficient to maintain the serum Br⁻ level to an amount between about 50 μM and about 1 mM is administered to the subject. For example, some embodiments of the invention can administer only the amount of Br⁻ or Br-comprising compositions needed to maintain a subject's serum Br⁻ level about 50 μM. In another example, an embodiment can administer only the amount of Br⁻ or Br-comprising compositions needed to maintain a subject's serum Br⁻ level about 100 μM. In still another example, an embodiment can administer only the amount of Br⁻ or Br-comprising compositions needed to maintain a subject's serum Br⁻ level about 250 μM. In yet another example, an embodiment can administer only the amount of Br⁻ or Br-comprising compositions needed to maintain a subject's serum Br⁻ level about 500 μM. In yet another example, an embodiment can administer only the amount of Br⁻ or Br-comprising compositions needed to maintain a subject's serum Br⁻ level about 750 μM. Embodiments can administer only the amount of Br⁻ or Br-comprising compositions needed to maintain a subject's serum Br⁻ level above about 20 μM, for example above 50 μM, yet below about 1 mM. Thus, embodiments of the invention can provide a safe yet effective amount of Br⁻ to a subject.

Embodiments of the invention are useful for the administration of Br⁻ and Cl⁻ ions in combination to a subject. Such embodiments represent an advancement in the pharmacologic administration of Br⁻ to subject in that superior dosing control can be provided over the final circulating concentrations of Br⁻ in a subject. Previous and historical administration of Br⁻ were plagued by a Br⁻ half-life in humans that in some circumstances was 7 days or longer due to renal reabsorption of Br³¹ . This extended half-life created complications in achieving and maintaining a desired serum or plasma level of Br⁻ in a subject, with high risk that circulating levels of Br⁻ would accumulate to toxic levels. Embodiments of the invention provide a pharmacologic strategy to better control the administration of Br⁻ by enhancing the rate renal excretion of Br³¹ . Specifically, co-administered Cl⁻ ions enhance renal excretion of Br⁻ which allows the subject to be administered an effective amount of Br⁻ with minimal risk of Br⁻ accumulation.

Embodiments of the invention comprise a pharmaceutical composition that comprises an amount of Br⁻ and Cl⁻ ions wherein the molar ratio of Br⁻ to Cl⁻ is between about 0.0338% and about 4.613%. For example, some embodiments of the invention comprise a molar ratio that is about 0.0338%, 0.04%, 0.05%, 0.06%, 0.07%, 0.08%, 0.09%, 0.10%, 0.12%, 0.14%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, 1.00%, 1.02%, 1.04%, 1.06%, 1.08%, 1.10%, 1.12%, 1.14%, 1.16%, 1.18%, 1.20%, 1.22%, 1.24%, 1.26%, 1.28%, 1.30%, 1.32%, 1.34%, 1.36%, 1.38%, 1.40%, 1.42%, 1.44%, 1.46%, 1.48%, 1.50%, 1.52%, 1.54%, 1.56%, 1.58%, 1.60%, 1.62%, 1.64%, 1.66%, 1.68%, 1.70%, 1.72%, 1.74%, 1.76%, 1.78%, 1.80%, 1.82%, 1.84%, 1.86%, 1.88%, 1.90%, 1.92%, 1.94%, 1.96%, 1.98%, 2.00%, 2.02%, 2.04%, 2.06%, 2.08%, 2.10%, 2.12%, 2.14%, 2.16%, 2.18%, 2.20%, 2.22%, 2.24%, 2.26%, 2.28%, 2.30%, 2.32%, 2.34%, 2.36%, 2.38%, 2.40%, 2.42%, 2.44%, 2.46%, 2.48%, 2.50%, 2.52%, 2.54%, 2.56%, 2.58%, 2.60%, 2.62%, 2.64%, 2.66%, 2.68%, 2.70%, 2.72%, 2.74%, 2.76%, 2.78%, 2.80%, 2.82%, 2.84%, 2.86%, 2.88%, 2.90%, 2.92%, 2.94%, 2.96%, 2.98%, 3.00%, 3.02%, 3.04%, 3.06%, 3.08%, 3.10%, 3.12%, 3.14%, 3.16%, 3.18%, 3.20%, 3.22%, 3.24%, 3.26%, 3.28%, 3.30%, 3.32%, 3.34%, 3.36%, 3.38%, 3.40%, 3.42%, 3.44%, 3.46%, 3.48%, 3.50%, 3.52%, 3.54%, 3.56%, 3.58%, 3.60%, 3.62%, 3.64%, 3.66%, 3.68%, 3.70%, 3.72%, 3.74%, 3.76%, 3.78%, 3.80%, 3.82%, 3.84%, 3.86%, 3.88%, 3.90%, 3.92%, 3.94%, 3.96%, 3.98%, 4.00%, 4.02%, 4.04%, 4.06%, 4.08%, 4.10%, 4.12%, 4.14%, 4.16%, 4.18%, 4.20%, 4.22%, 4.24%, 4.26%, 4.28%, 4.30%, 4.32%, 4.34%, 4.36%, 4.38%, 4.40%, 4.42%, 4.44%, 4.46%, 4.48%, 4.50%, 4.52%, 4.54%, 4.56%, 4.58%, 4.60%, about 4.613%, or any other amount that is between about 0.0338% and about 4.613% of the amount of Cl⁻ on a molar basis. In some embodiments, the Br⁻ and Cl⁻ ions are formulated as any pharmaceutically acceptable salts. In some embodiments, the Br⁻ and Cl⁻ ions are formulated as pharmaceutically acceptable salts of Na⁺, K⁺, Mg²⁺, Ca²⁺, or any combination thereof. In some embodiments, the composition is formulated as a pill, patch, strip, or liquid. In some embodiments, the composition is administered to a subject via oral, transdermal, injection, rectal, inhalation, nasal, or a combination thereof, routes of administration.

A particular advantage of embodiments of the invention is its use of the molar ratio of Br⁻ to Cl⁻. While many, if not most, nutritional and therapeutic compositions utilize units of mass or volume, such as grams or milliliters, embodiments of the invention utilize the molar ratio of Br⁻ to Cl⁻. The benefits of molar ratio are realized upon considering that Cl⁻ are added, frequently by weight, to numerous pharmaceutical, saline, nutritional, and food compositions. Thus, when embodiments of the invention are used in place of standard Cl⁻ reagents or ingredients, the molar basis of embodiments of the invention provides a convenient means for ensuring that a constant proportion of Br⁻ is added to the composition. This allows the final pharmaceutical, saline, nutritional, or food composition to contain a nutritional or therapeutic amount of Br⁻ by simply adding the desired amount of Cl⁻. For example, a meal can be fortified with a nutritional amount of Br⁻ when one simply salts his or her food to taste using a table salt embodiment of the invention. As another example, a pharmaceutical composition can administer a physiologically-balanced amount of Br⁻ and Cl⁻ to a subject by administering an embodiment of the invention that contains a therapeutically effective amount of Cl⁻.

Embodiments of the invention comprise a method of treating or preventing disease in a subject, the method comprising administering a therapeutically effective amount of a pharmaceutical composition comprising an amount of Br⁻ and Cl⁻ ions wherein molar amount of Br⁻ an amount between about 0.0338% and about 4.613% of the molar amount of Cl⁻. In some embodiments, the disease comprises cardiovascular disease, hypertension, kidney disease, end stage renal disease, diabetes, metabolic syndrome, obesity, cancer, an eye disease, an ear disease, a wound, an ulcer, an infection, inflammation, a degenerative disease, a skin disease, cystic fibrosis, or a combination thereof.

Some subjects may display serum Br⁻ levels below about 50 μM. Embodiments of the invention can be used to administer nutritionally effective amounts of Br⁻ to said subjects, resulting in serum Br⁻ levels that are between 50 μM and 1 mM after administration.

### Extracellular Matrix (ECM)

Extracellular matrices (ECM) are found throughout tissues and provide essential mechanical and signaling support for tissues. An example of an ECM is the basement membrane (BM). These matrices can exhibit a perturbed structural appearance during disease. For example, structural changes within the vascular walls can be seen in patients with end-stage renal disease who are at risk of developing cardiovascular disease (Bevc et al., 2006). As another example, thickening of the heart ventricular wall can be seen in some patients with heart disease. Embodiments of the invention can be useful in maintaining proper ECM structure in order to promote heart health, vascular health, and/or kidney health in subjects.

### Connections Between Heart, Kidneys, and Vasculature

There is much evidence that heart, kidney, and vascular physiology are interrelated. Patients with kidney disease have an elevated risk of developing various forms of cardiovascular disease. Conversely, for example, hypertension is a leading cause of kidney failure. Fundamentally, the heart and kidneys are both physically connected by the vasculature and both functionally interact with the bloodstream, where the heart pumps blood while the kidneys filter blood. Thus, the inventors believe that this interrelated nature allows the invention to promote heart health, kidney health, as well as vascular health.

Elevated dietary salt (e.g. NaCl, sodium chloride) intake is associated with poor cardiovascular and kidney health. As a result, the general public is often advised to consume a low-sodium or low-salt diet. As a new concept in nutritional science, the invention is built on the discovery that a nutritionally effective amount of Br⁻ is able to counteract the negative health effect of excess NaCl. Accordingly, the invention provides an unexpected advance to this long-held medical wisdom about the health dangers of too much salt. Specifically, embodiments of the invention use Br⁻ ions to reduce the medical risks that are known to be associated with a diet that is rich in NaCl or sodium. Importantly, during practice of the invention, this effect of Br⁻ can be accomplished while the subject consumes a high-salt diet. For example, in one embodiment of the invention, a nutritionally effective amount of Br⁻ is administered to a patient with salt-sensitive hypertension who regularly consumes a high sodium diet. In this particular example, practice of the invention could prophylactically mitigate some of the health risks that stem from the patient's unhealthy diet without actually requiring additional changes to the diet beyond practice of the invention itself. While it would of course be desirable for said patient to adopt a low salt diet, it can be challenging for some individuals to successfully modify their diet. With this in mind, the invention is capable of promoting heart health, kidney health, and/or vascular health even when patients do not adopt healthy dietary modifications.

In rats, the excretion rate of Br⁻ has been shown to be dependent on sodium Na⁺ excretion (Pavelka et al., 2005). Moreover, it is known that Na⁺ excretion rates are enhanced when excess Na⁺ is consumed. Thus, rats that consume elevated amounts of Na⁺ will begin excreting greater amounts of Na⁺ and Br⁻, resulting in a lowering of circulating levels of Na⁺ and Br⁻. In humans, excess consumption of NaCl will cause circulating Br⁻ levels to fall. Thus, consumption of a high-salt diet can deplete a subject of adequate circulating Br⁻. Such a fall in circulating Br⁻ has been widely, yet incorrectly, understood to be beneficial to the health of a subject, in accordance with the popular misunderstanding that Br⁻ is “toxic”. In contrast, the invention utilizes nutritionally effective amounts of Br⁻ to promote heart health, kidney health, and/or vascular health in a subject.

There is an observed clinical association between elevated sodium consumption and risk of developing hypertension, vascular disease, other forms of cardiovascular disease, chronic kidney disease, and other forms kidney disease. Embodiments of the invention can be used to treat the diseases described herein, and improve heart health, kidney health, and/or vascular health in subjects with these diseases.

Similar to salt-induced Br⁻ depletion, Br⁻ depletion may occur in some subjects who take thiazide or “water pills”. These drugs act by enhancing sodium clearance rates, and can simultaneously enhance Br⁻ clearance rates. The invention can be useful for administering nutritionally effective amounts of Br⁻ to subjects who receive these drugs, in order to promote heart health, kidney health, and/or vascular health.

For patients treated with maintenance dialysis, dialysis fluids contain lower amounts of Br⁻ than is found in a subject's bloodstream, causing bloodstream Br⁻ to transfer to the dialysis fluid (from high to low concentrations) during the dialysis treatment and effectively removing dietary-derived Br⁻ from the subject. Thus, dialysis patients are exposed to chronically low Br⁻ levels. Such patients are at high risk of developing cardiovascular disease, anemia, stroke, peripherial artery disease, and other co-morbidities. Embodiments of the invention can be used to administer nutritionally effective amounts of Br⁻ to patients on dialysis.

These disease mechanisms apply to human as well as animal health. As such, embodiments of the invention can be used to administer nutritiously effective amounts of bromide (Br⁻) to humans, dogs, cats, mouse, rabbit, rat, hamster, guinea pig, horses, cows, goats, sheep, pigs, chickens, or turkeys. Similar to humans, animals can experience poor heart health, kidney health, and/or vascular health. For example, cats are prone to develop heart disease, specifically feline hypertrophic cardiomyopathy, while dogs are prone to kidney disease, specifically canine chronic kidney disease. As another example, some cats are prone to develop kidney disease while some dogs are prone to heart disease. As still other examples, pulmonary hypertension can cause heart failure in cows and chickens, Mulberry heart disease can affect pigs, while turkeys can experience sudden death due to cardiovascular causes, all of which can cause severe economic harm during the raising and production of animals for. Embodiments of the invention can be useful for promoting heart health, kidney health, and/or vascular health in animals including but not limited to dogs, cats, mouse, rabbit, rat, hamster, guinea pig, pigs, cows, sheep, goats, horses, chickens, and turkeys.

Collagen IV biosynthesis requires ascorbic acid (vitamin C), and as such, individuals with combined deficiency of vitamin C and Br⁻, or at least chronically low levels of both, can exhibit widespread defects in their tissues. For such an individual, embodiments comprise compositions comprising Br⁻ and an appropriate amount of vitamin C. Such compositions can effectively administer both Br⁻ and vitamin C to a subject in need thereof, such as one with chronically low circulating levels of both Br⁻ and vitamin C.

Br⁻ depletion can occur in combination with magnesium (Mg²⁺) deficiency. Dialysis patients can be susceptible to this combination deficiency, owing to the nutritional imbalances created by dialysis treatments. Mg-deficiency is associated with increased risk of mortality in dialysis patients (Sakaguchi et al., 2013). Patients with serum Mg²⁺ levels between 2.5 and 3.0 mg/dl have shown the lowest risk of developing cardiovascular disease (Sakaguchi et al., 2013). In some patients with hyperphosphatemia, low Mg²⁺ levels can promote risk of calcification, including vascular calcification (Sakaguchi et al., 2014). The occurrence of low Br⁻ and Mg-deficiency in the same patient, such as a dialysis patient, can further elevate the patient's risk of mortality, cardiovascular disease, or vascular calcification. Embodiments comprise compositions comprising Br⁻ and an appropriate amount of Mg²⁻. Such compositions can effectively administer both Br⁻ and Mg²⁺ to a subject in need thereof, such as one with chronically low circulating levels of both Br⁻ and Mg²⁺.

Anemia is a symptom of iron deficiency and can also occur in subjects who are in need of Br⁻ administration through practice of the invention. For example, dialysis can lower the Br⁻ levels in a patient as well as increase risk of developing anemia. Embodiments comprise compositions comprising Br⁻ and an appropriate amount of iron. Such compositions can effectively administer both Br⁻ and iron to a subject in need thereof, such as one with chronically low circulating levels of both Br⁻ and iron.

B vitamins are import for kidney and heart health as well as numerous cellular and metabolic functions. This vitamin class is comprised of many different chemical species, including thiamine, riboflavin, niacin, pantothenic acid, pyridoxine, pyridoxal, pyridoxamine, biotin, folic acid, and cobalamin. Embodiments comprise compositions comprising Br⁻ and an appropriate amount of B vitamins, B vitamin derivatives, or a combination thereof. Such compositions can effectively administer both Br⁻ and B vitamins and/or B vitamin derivatives to a subject in need thereof, such as one with chronically low circulating levels of both Br⁻ and B vitamins.

### Biomarkers of Cardiovascular Disease and Kidney Disease

Embodiments of the invention can be used to identify a subject who would benefit from administration of an amount of Br⁻ or Br-comprising composition sufficient to maintain the serum Br⁻ level to an amount between about 20 μM and about 1 mM, for example between about 50 μM and about 1 mM. In some embodiments, said subject displays one or more of the following: systolic blood pressure that displays a clinically meaningful difference compared to baseline levels, healthy levels, normal levels, or medically acceptable systolic blood pressure reference values; diastolic blood pressure that displays clinically meaningful difference compared to baseline levels, healthy levels, normal levels, or medically acceptable diastolic blood pressure reference values; heart rate that displays a clinically meaningful difference compared to baseline levels, healthy levels, normal levels, or medically acceptable heart rate reference values; cardiac fibrosis; salt sensitive hypertension; glomerular filtration rate less than 60 ml/min/1.73 m2; urine albumin levels that display a clinically meaningful elevation over baseline levels, healthy levels, normal levels, or medically acceptable reference values; serum levels of BNP, ANP, or pro-BNP that display a clinically meaningful difference compared to baseline levels, healthy levels, normal levels, or medically acceptable reference values; or urinary or plasma cyclic guanosine monophosphate (cGMP) levels that display a clinically meaningful difference compared to baseline levels, healthy levels, normal levels, or medically acceptable reference values.

Hypertension is a well-known cause of many forms of cardiovascular and kidney diseases, including but not limited to heart attack, heart failure, atherosclerosis, chronic kidney disease, and end stage renal disease. For patients presenting with elevated blood pressure, a key clinical objective is achieving sustained reduction of blood pressure through lifestyle modifications, dietary modifications, and/or the prescription of hypertensive medications. Non-limiting examples of lifestyle and dietary modifications include adopting a daily exercise routine; avoiding caffeine; avoiding alcohol; and increasing dietary intake of fiber, nuts, and fresh fruits and vegetables. Non-limiting examples of hypertensive medications include thiazide diuretics, potassium sparing diuretics, loop diuretics, angiotensin converting enzyme inhibitors, angiotensin receptor blockers, β-blockers, vasodilators, calcium channel blockers, a-blockers, renin inhibitors, aldosterone antagonists, alpha2-agonists, and adrenergic agents. Embodiments of the invention can be used as food items or medical foods that may assist in controlling a subject's blood pressure. Other embodiments of the invention can be used as hypertensive medications that therapeutically maintain reduced blood pressure levels in a subject. Embodiments of the invention can be used in isolation or in combination with lifestyle modifications, dietary modifications, and/or other hypertensive medications.

The American Heart Association (AHA) recommends that normal blood pressure values comprise a systolic blood pressure below 120 mm Hg and a diastolic blood pressure below 80 mm Hg. Low blood pressure can be defined as systolic blood pressure lower than 90 mm Hg and/or diastolic lower than 60 mm Hg. The AHA defines prehypertension as either a systolic blood pressure between 120-139 mm Hg or a diastolic blood pressure between 80 and 89 mm Hg. Further, the AHA defines Stage 1 Hypertension as either a systolic blood pressure between 140-159 mm Hg or a diastolic blood pressure between 90 and 99 mm Hg. The AHA defines Stage 2 Hypertension as either a systolic blood pressure of at least 160 mm Hg or a diastolic blood pressure of at least 100 mm Hg. Finally, the AHA defines a hypertensive emergency as either a systolic blood pressure of above 180 mm Hg or a diastolic blood pressure of above 110 mm Hg (http://www.heart.org/HEARTORG/Conditions/HighBloodPressure/KnowYourNumbers/Un derstanding-Blood-Pressure-Readings_UCM_301764_Article.jsp). These numbers are reference values to be used by a medical professional, such a nurse, doctor, physician, or physician assistant, in the clinical evaluation of a subject. For example, a doctor, physician, physician assistant, or other qualified medical professional may determine that a patient has stage 1 hypertension if the patient displays a systolic blood pressure of 145 mm Hg and a diastolic blood pressure of 95 mm Hg. As another example, a subject may be at risk of developing hypertension if the subject's blood pressure rises from 110 over 75 (systolic over diastolic) mm Hg to 140 over 90 mm Hg over a 3 month period and the subject's blood pressure is continuously elevated for the next 3 months. Embodiments of the invention can be used to lower blood pressure in a subject.

Importantly, embodiments of the invention only use small amounts of Br⁻ to achieve significant reductions in blood pressure. Specifically, embodiments of the invention only administer an amount of Br⁻ or Br-comprising composition sufficient to maintain the serum Br⁻ level to an amount between about 50 μM and about 1 mM in the subject. This distinguishes embodiments of the invention from the historical pharmacologic administration of Br⁻ that suggested large doses of Br⁻ are capable of lowering arterial blood pressure (Addison, Can. Med. Assoc. J, 18(3): 281-285, 1928). The historical reference used excessive amounts Br⁻ to achieve the reported reduction in blood pressure, thereby placing the patient at risk of developing a Br-related toxicity. As a result, the therapeutic potential of Br⁻ as a blood pressure treatment has been ignored. Embodiments of the invention enable Br⁻ to be safely administered as a blood pressure lowering treatment to a subject, overcoming the toxicity risks that limited prior pharmacologic use of Br⁻.

Embodiments of the invention may reduce blood pressure in a subject by triggering slowing of the subject's heart rate. Elevated peripheral heart rate is associated with hypertension and the development of cardiovascular disease (Reule & Drawz, Curr. Hypertens. Rep. 2012). The AHA recommends an average resting heart rate of between 60-100 beats per minute for individuals age 10 years old and older (http://www.heart.org/HEARTORG/HealthyLiving/PhysicalActivity/FitnessBasics/Target-Heart-Rates_UCM_434341_Article.jsp). However, while heart rate is routinely monitored in various emergency medicine, clinical, and hospital settings, heart rate reducing medications are not associated with reducing adverse events in hypertensive patients. In contrast, embodiments of the invention can reduce adverse events in a hypertensive subject as demonstrated by the reduced morbidity experienced by salt sensitive Dahl rats receiving Br⁻ administration (FIG. 1).

Kidney function is commonly assessed using the glomerular filtration rate (GFR). Moreover, the staging of kidney disease is determined by the GFR of a patient where, in broad terms, a GFR between 120 to 60 is normal, a GFR between 60 and 15 represents kidney disease, while a GFR below 15 represents kidney failure. More specifically, stage 1 chronic kidney disease (CKD) can be defined as a GFR of 90 or above, stage 2 CKD can be defined as a GFR between 89-60, stage 3a CKD can be defined as a GFR between 59-44, stage 3b CKD can be defined as a GFR between 44-30, stage 4 CKD can be defined as a GFR between 29-15, and stage 5 CKD (“kidney failure”) can be defined as a GFR below 15 (https://www.kidney.org/atoz/content/gfr). Subjects with kidney disease, as determined by a GFR of 60 or below, have an increased risk of cardiovascular disease. Conversely, subjects with cardiovascular disease, such as but not limited to hypertension, are at increased risk of developing kidney disease that is evidenced by a decline in their GFR. Embodiments of the invention can be administered to a subject with a GFR of 60 or below. Some embodiments of the invention can be administered to a subject with a GFR of 60 or above and who are at risk of developing kidney disease such as individuals with diabetes, existing cardiovascular disease, hypertension, a family history of kidney disease, or who display concurrent albuminuria.

The presence of albumin in urine can indicate a decline in kidney function. Within the tubules of healthy kidneys, most of the albumin that is filtered through the glomerulus is actively removed from filtrate. As a result, the presence of albumin in urine can indicate pathologic disruption of either glomerular or tubular activity. Urinary albumin levels are often expressed as the ratio of albumin to creatinine in urine, where a normal or healthy ratio is 30 mg albumin per gram of creatinine (30 mg/g). A ratio between 30-299 mg/g can represent moderately increased albuminuria. A ratio above 300 mg/g can represent severely increased albuminuria.

Natriuretic peptide (NP) signaling is involved with cardiovascular and renal diseases. Elevated serum levels of brain natriuretic peptide (BNP) are a diagnostic marker of acute decompensated heart failure. In patients with acute dyspnea, BNP levels over 400 pg/ml indicate heart failure, levels below 100 indicate heart failure is unlikely, and levels between 100-400 require the clinical judgement of the attending physician or doctor in order to be correctly diagnosed as heart failure. Additional diagnostic value can be found in the N-terminal of proBNP (NT-proBNP), which is precursor protein of BNP. In patients younger than 50 years of age with acute dyspnea, NT-proBNP levels over 450 pg/ml are indicative of heart failure. In patients between 50 and 75 years with acute dyspnea, NT-proBNP levels over 900 are indicative of heart failure. In patients older than 75 years with acute dyspnea, NT-proBNP levels over 1800 are indicative of heart failure. Moreover, elevated levels of BNP and atrial natriuretic peptide (ANP) can be seen in patients with chronic kidney disease. Healthy individuals can display blood ANP levels between 22-77 pg/ml. In embodiments, blood levels of natriuretic peptides, such as ANP and BNP can indicate the need for administration of Br-compositions as described herein, where BNP levels below about 50 pg/ml or above 100 pg/ml or where ANP levels above about 77 pg/ml or below about 22 pg/ml can indicate need for administration of an embodiment of the invention. In some embodiments, BNP levels below about 40 pg/ml, or below about 30 pg/ml, or even below about 20 pg/ml, or even still below about 10 pg/ml can indicate need for administration of an embodiment of the invention. In some embodiments, BNP levels above about 100 pg/ml, or above about 200 pg/ml, or above about 300 pg/ml, or above about 400 pg/ml, or above about 500 pg/ml, or above about 600 pg/ml, or above about 700 pg/ml, or above about 800 pg/ml, or above about 900 pg/ml, or above about 1000 pg/ml, or above about 1100 pg/ml, or above about 1200 pg/ml, or above about 1300 pg/ml, or above about 1400 pg/ml, or above about 1500 pg/ml, or above about 1600 pg/ml, or above about 1700 pg/ml, or above about 1800 pg/ml, or above about 1900 pg/ml, or above about 2000 pg/ml can indicate need for administration of an embodiment of the invention. In some embodiments, ANP levels below about 20 pg/ml, or below about 15 pg/ml, or below about 10 pg/ml, or even below about 5 pg/ml, or even below about 1 pg/ml, or even still undetectable amounts of ANP can indicate need for administration of an embodiment of the invention. In some embodiments, ANP levels above about 77 pg/ml, above about 80 pg/ml, above about 90 pg/ml, above about 100 pg/ml, above about 110 pg/ml, above about 120 pg/ml, above about 130 pg/ml, above about 140 pg/ml, above about 150 pg/ml, above about 160 pg/ml, above about 170 pg/ml, above about 180 pg/ml, above about 190 pg/ml, above about 200 pg/ml, above about 210 pg/ml, above about 220 pg/ml, above about 230 pg/ml, above about 240 pg/ml, above about 250 pg/ml, above about 260 pg/ml, above about 270 pg/ml, above about 280 above about 290 pg/ml, or even above about 300 can indicate need for administration of an embodiment of the invention.

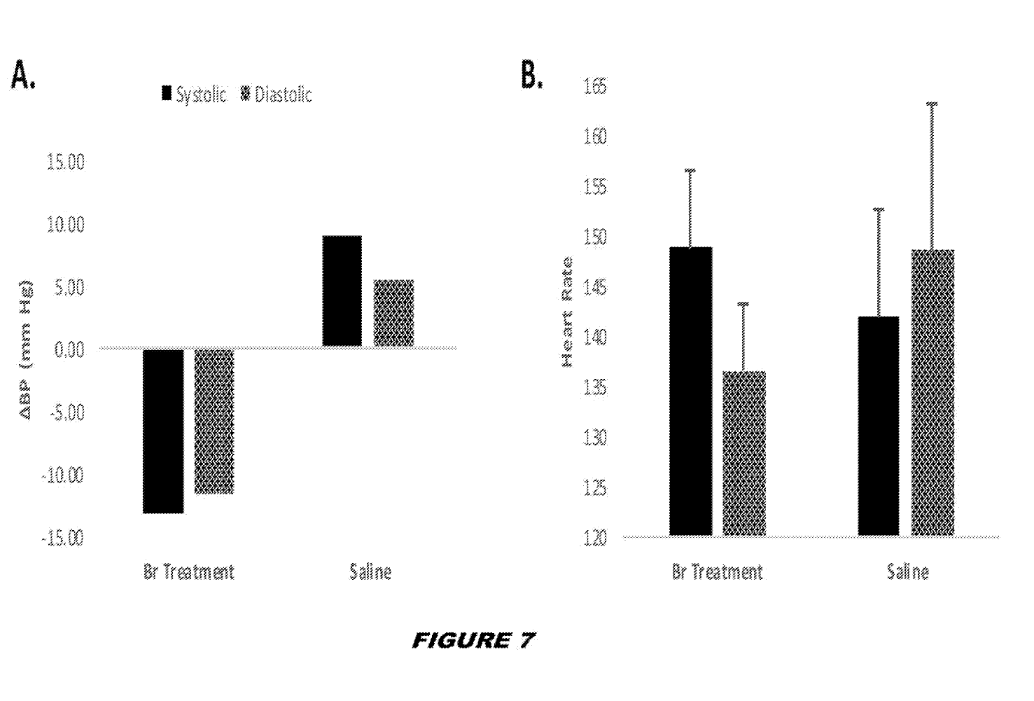

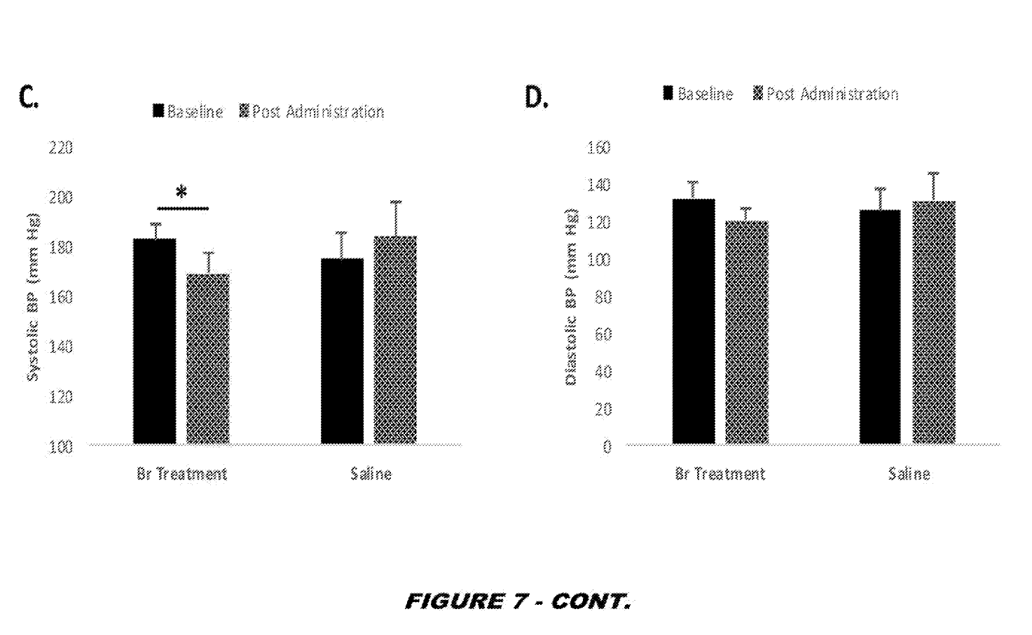

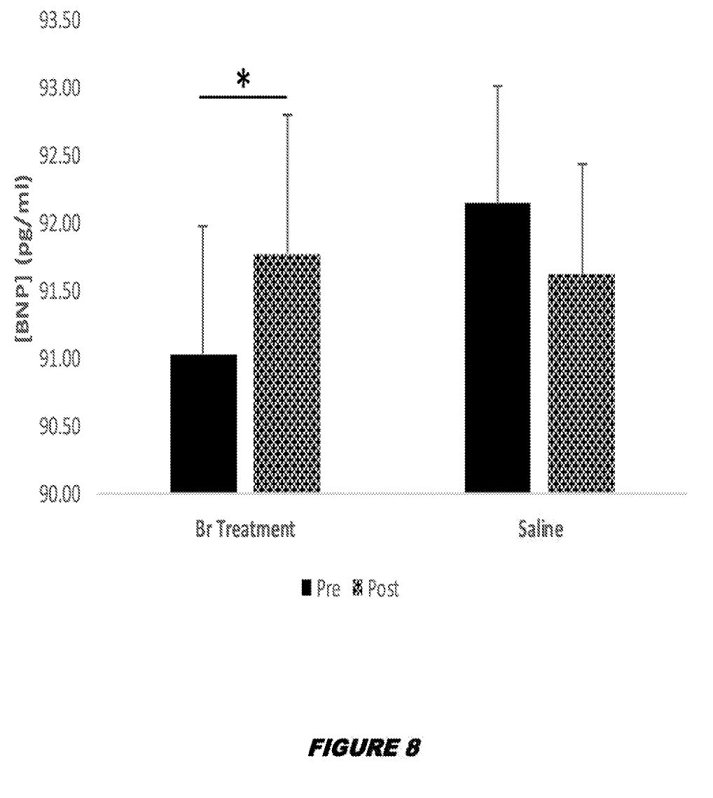

Without being bound by theory, NP's can be therapeutic, for example, certain recombinant forms of ANP and BNP have been developed as drugs such as carperitide (recombinant ANP) and nesiritide (recombinant BNP). However, the clinical performance of these NP-derived pharmaceuticals has not matched their hypothesized potential. For example, carperitide administration is associated with increased in-hospital mortality in acute heart failure patients in Japan (Matsue et al., J. Card. Fail., 21(11):859-864, 2015). As another example, nesiritide administration has been associated with worsening renal function and increased mortality in patients with acute decompensated heart failure (Sackner-Bernstein et al., Circulation, 111(12):1487-1491, 2005; Sackner-Berstein et al., JAMA, 293(15):1900-1905, 2005). Without being bound by theory, the field of heart failure has been long plagued by therapeutics that improve one or more biomarkers of disease, or surrogate end points, while not significantly improving long-term clinical outcomes such as survival or hospitalization rates. As discussed herein, Br⁻ is an important component of NP signaling (FIGS. 7-8), and Br-serves along with ANP as well as BNP as improved biomarkers of disease as well as clinical outcomes. Specifically, embodiments of the invention can modulate NP expression, reduce blood pressure, reduce cardiac fibrosis, and increase survival by way of regulating Br-levels (FIGS. 1,6-8).

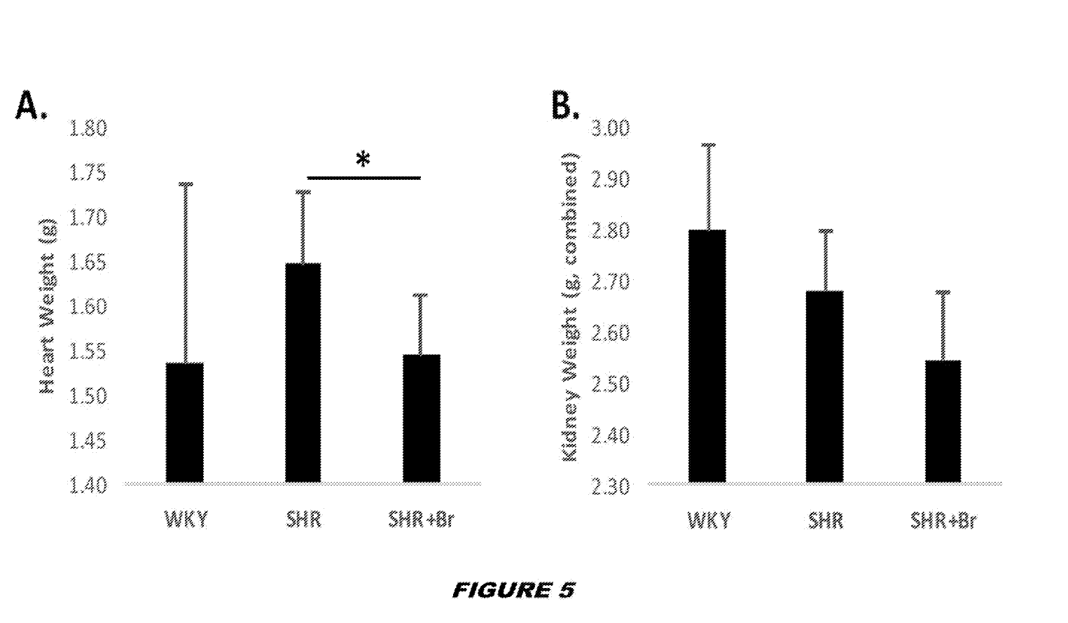

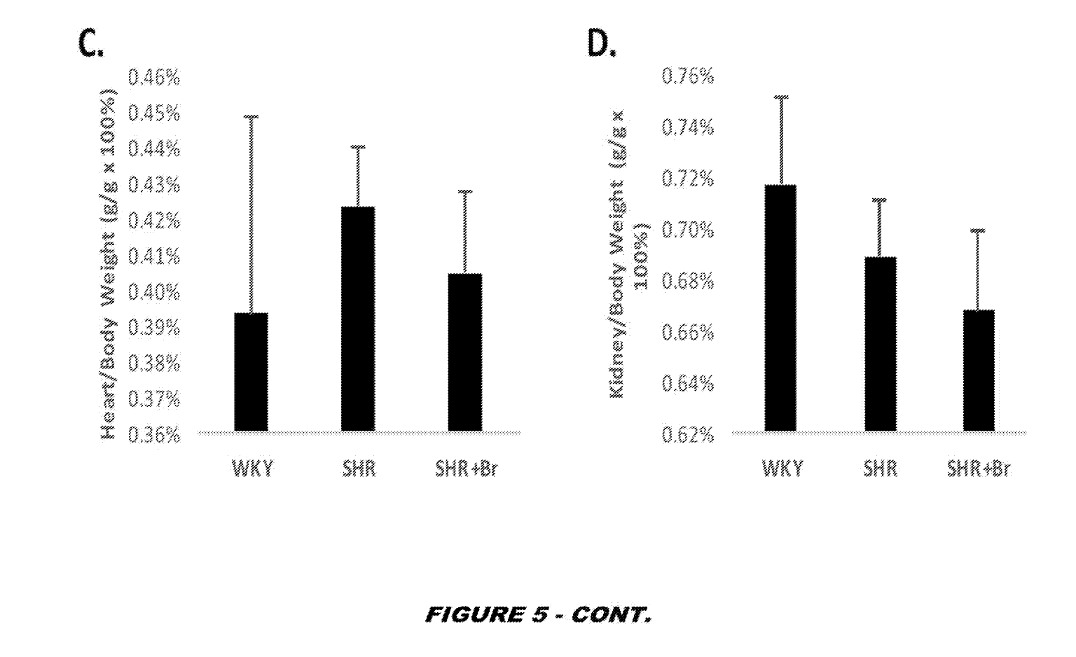

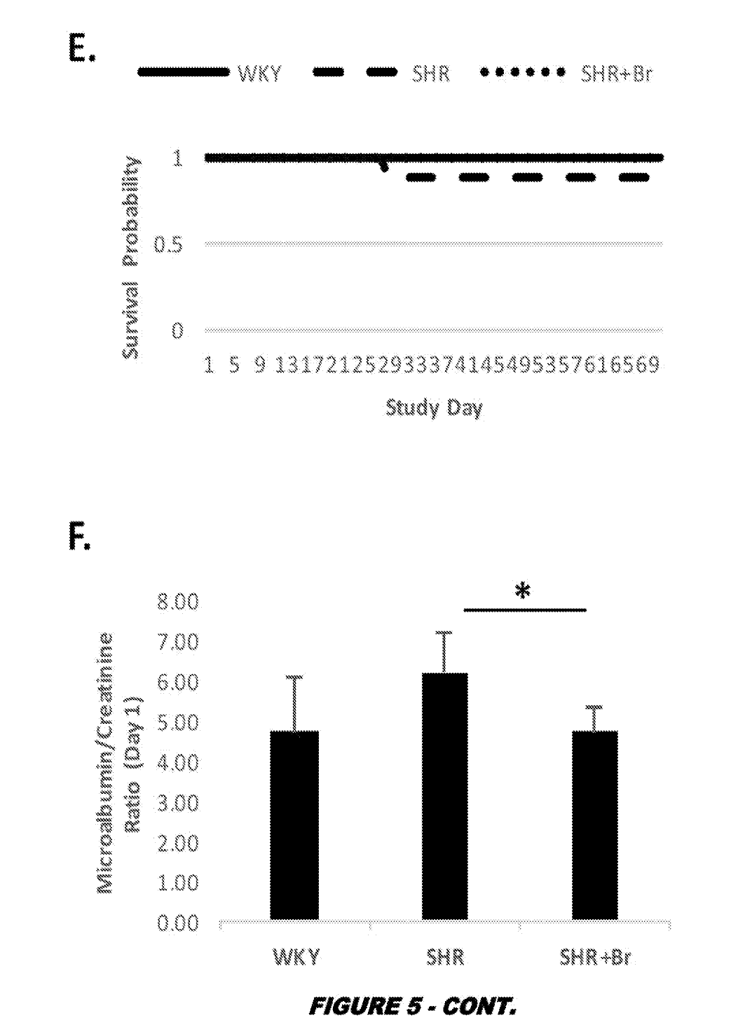

ANP and BNP both utilize cyclic guanosine monophosphate (cGMP) as a second messenger molecule. Healthy individuals can display plasma cGMP levels ranging from 0.1-3.0 pg/ml. Thus, when compared to healthy individuals, increased concentrations of cGMP in plasma or urine can indicate ongoing activity of natriuretic peptide signaling. Moreover, patients with heart failure are reported to display decreased ratios of cGNP to BNP in plasma and urine (Lourenco et al., Eur. J Heart Fail., 2009), suggesting that there is a critical blockage of signal transduction from BNP to active cGMP. BNP signaling is known to trigger natriuresis and reduce blood pressure, in agreement with the inventors data (FIGS. 5,6). Without wishing to be bound by theory, when practiced as taught herein, embodiments of the invention can modulate the natriuretic peptide signaling system so as to achieve therapeutic reductions in blood pressure. Thus, embodiments as described herein comprise natriuretic peptides, such as ANP and BNP, as bio-indicators for the effectiveness of Br-containing compositions as described herein. Further, natriuretic indicators, such as ANP and BNP, can serve as prognostic and/or diagnostic indicators of a subject in need of a Br-containing composition. For example, ANP and/or BNP levels in a subject can be measured, such as an obese subject or a subject with diabetes, and the subject can be identified as one in need of a Br-containing composition if such levels of ANP and/or BNP are suppressed when compared to physiologically normal levels.

Cardiac fibrosis can develop in a subject as a consequence of cardiovascular disease, such as but not limited to hypertension and myocardial infarction. During fibrosis, cardiac tissue accumulates an excessive amount of collagen fibers. This is sometimes referred to as developing “scar tissue” inside the heart, effectively stiffening the tissue and increasing a subject's risk of developing heart failure. Types I, III, and VI collagen are frequently associated with cardiac fibrosis, and in particular collagens VI and III are suggested by some to be involved with the development of cardiac fibrosis. Collagen IV is also found in cardiac tissue and is reportedly disrupted in some heart failure patients, although collagen IV is present as a minor collagen component in heart tissue. The “gold standard” for diagnosing fibrosis is a biopsy. Other techniques for evaluating cardiac fibrosis include but are not limited to echocardiography; cardiac magnetic resonance; and measuring serum biomarkers such as but not limited to N-terminal collagen I propeptide, serum N-terminal collagen III propeptide, and C-terminal collagen I telopeptide where increases in these biomarkers is correlated with remodeling or fibrosis. Embodiments of the invention can prevent or treat cardiac fibrosis in a subject.

Clinically meaningful changes in a biomarker such as but not limited to blood pressure, heart rate, eGFR, or albuminuria can be readily determined by a doctor or physician who monitors a subject over a period of time, such as but not limited to 1 month, 3 months, 6 months, 9 months, 1 year, or longer. In some instances, a change may be associated with another event in the subject's life, such as but not limited to the development of hypertension during pregnancy or the decline in eGFR in a subject with a history of hypertension. In some instances, a change may be transitory such as but not limited to a temporary increase in urinary albumin levels followed by a return to normal urine albumin levels. In other instances, changes may occur in two or more biomarkers such as but not limited to a subject to develops hypertension and an elevated resting heart rate. Medical societies and authorities such as but not limited to the American Heart Association, the Center for Disease Control, the Blood Pressure Association, the National Kidney Foundation, and the National Institute for Health, can issue recommendations or guidelines for determining ideal, healthy, or normal biomarker levels. The clinical judgement of a doctor or physician can determine whether a particular change in one or more biomarkers is clinically meaningful.

Upon administration to a subject, various biomarkers can be used to monitor the safety, activity, and efficacy of embodiments of the invention. Safety of embodiments can be directly assessed through monitoring the amount of Br— in a subject, such as but not limited to monitoring the amount of Br— in blood, plasma, serum, urine, hair, nails, saliva, sweat, spent or used dialysate, or cells; determining the average concentration of Br— in circulation, such as from blood, serum, or plasma; and determining that the use of the embodiment is safe if the average concentration of Br— is below 1 mM. Activity of the embodiments can be monitored by examining disease biomarkers associated with NP signaling, such as but not limited to blood pressure, heart rate, BNP levels, ANP levels, or cGMP levels. Activity of the embodiment would be evidenced by reductions in blood pressure or heart rate or elevations in BNP, ANP, or cGMP in blood or urine. At the level of an individual subject, efficacy of embodiments of the invention can be evaluated by monitoring the degree of fibrosis in a subject who is treated with an embodiment. In a population, such as in a clinical trial, efficacy of embodiments of the invention can be evaluated by monitoring mortality rates or hospitalization rates.

Without being bound by theory, biomarkers can be used to assess the health of a subject. As a non-limiting example, systolic blood pressure is considered to be normal or healthy when it is between about 90 mm Hg and about 120 mm Hg. Without being bound by theory, while a subject with a systolic blood pressure of 130 mm Hg may be diagnosed as prehypertensive, another subject with a systolic blood pressure of 119 mm Hg may be considered “boarder line” prehypertensive or at the upper range of normal. Nutritional embodiments can be used to help a subject maintain one or more biomarkers within a healthy range. In some embodiments, the biomarker and its healthy level or range is selected from serum, blood, or plasma Br-concentration between about 30 μM and about 1 mM; systolic blood pressure between about 90 mm Hg and about 120 mm Hg; diastolic blood pressure between about 60 mm Hg and about 80 mm Hg; GFR above about 60; resting heart rate between about 60 and about 100 beats per minute; and urinary albumin levels below about 30 mg/g.

Without being bound by theory, embodiments of this method can identify a subject in need of a dietary item that is beneficial for heart health, kidney health, and or vascular health by (1) using a diagnostic test to measure the serum, blood, salivary, or urinary biomarkers levels of lipids, cholesterol, high-density lipoprotein, low-density lipoprotein, very-low-density lipoprotein, homocysteine, creatinine, c-reactive protein, cystatin C, tryglyceride, protein, albumin, lipid oxidation, protein oxidation, kidney injury molecule 1, or neutrophil gelatinase-associated lipocalin; (2) for each biomarker examined, comparing the test result to the healthy range of said biomarker levels for the subject; (3) for each biomarker examined, calculating the absolute difference between the test result and the upper and lower boundaries of the healthy range; (4) for each biomarker examined, calculating the difference between the upper boundary and the lower boundary of the healthy range; (5) for each biomarker examined, calculating the ratio of the absolute difference found in Step 3 to the difference found in Step 4; and (6) for each biomarker examined, determining that the subject is in said poor health when the ratio calculated in Step 5 is less than or about 0.20. The two equations below illustrate the calculations described in Steps 2-6, where “Result” is the test result determined in Step 1, “Up” is the upper boundary of the healthy range of the biomarker of interest, and “Low” is the lower boundary of the healthy range of the biomarker of interest:

$$\frac{⌈ Test - Up ⌉}{( Up - Low )} \leq 0.20$$

$$or$$

$$\frac{⌈ Test - Low ⌉}{( Up - Low )} \leq 0.20$$

### Use of the Invention in Diabetes, Metabolic Syndrome, and Obesity

Risk of cardiovascular disease is increased if a subject is obese or has diabetes or metabolic syndrome. Embodiments of the invention can be administered to subjects who are obese or who have diabetes or metabolic syndrome. Some embodiments can prevent or treat cardiovascular disease in an obese subject or in a subject with diabetes or metabolic syndrome.

Pathologically, NP expression and signaling is suppressed in obesity, diabetes, and metabolic syndrome. In addition to controlling natriuresis and blood pressure, NP signaling activates hormonal pathways that influence metabolism including converting white adipose tissue to brown adipose tissue, triggering lipolysis in adipose tissue, increasing glucose-stimulated insulin secretion, and promoting lipid oxidation in skeletal muscle (Schlueter et al., Pharmacol. Ther., 144(1):12-27, 2014). Indeed, circulating levels of NPs are reduced in patients with obesity, diabetes, and metabolic syndrome (Schlueter et al., Pharmacol. Ther., 144(1):12-27, 2014). Interestingly, this reduction of NP levels may explain the association of hypertension with obesity, diabetes, and metabolic syndrome.

Embodiments of the invention can therapeutically administered to a subject with diabetes or metabolic syndrome. Embodiments of the invention can be therapeutically administered to an obese subject. Embodiments of the invention can be used to increase circulating ANP, BNP, or cGMP; promote ANP or BNP signaling; reduce blood pressure; prevent or treat diabetes, metabolic syndrome, or obesity in a subject. Embodiments of the invention can prevent or treat cardiovascular disease in a subject who is obese or who has diabetes or metabolic syndrome.

Embodiments of the invention can be administered alone to a subject, in combination with another pharmaceutical drug, as part of treatment regimen, or a component of a kit. In some embodiments, the other pharmaceutical drug is a drug used to treat diabetes, metabolic syndrome, or obesity, such as but not limited to insulin, amylinomimetic agents, alpha-glucosidase inhibitors, biguanides, dopamine agonists, glucagon-like peptides, meglitinides, sodium glucose transporter 2 inhibitors, sulfonylureas, thiazolidinediones, and dipeptidyl peptidase-4 inhibitors. In some embodiments, the drug comprises regular insulin such as but not limited to Humulin or Novolin, insulin aspart such as but not limited to Novolog or FlexPen; insulin glulisine such as but not limited to Apidra; insulin lispro such as but not limited to Humalog; insulin isophane such as but not limited to Humulin N or Novolin N; insulin degludec such as but not limited to Tresiba; insulin detemir such as but not limited to Levemir; insulin glargine such as but not limited to Lantus; insulin glargine such as but not limited to Toujeo; a combination insulin drug such as but not limited to insulin aspart protamine-insulin aspart, insulin lispro protamine-insulin lispro, human isophane insulin-human insulin regular, insulin dedludec-insulin aspart, NovoLog Mix 70/30, Humalog Mix 75/25, Humalog Mix 50/50, Humalin 70/30, Novolin 70/30, or Ryzodeg; pramlintide such as but not limited to SymlinPen; acarbose such as but not limited to Precose; miglitol such as but not limited to Glyset; metformin such as but not limited to Glucophage, Metformin Hydrochloride ER, Glumetza, Riomet, or Fortamet; a metformin-containing drug such as but not limited to metformin-alogliptin, Kazano, metformin-canagliflozin, Invokamet, metformin-dapagliflozin, Xigduo XR, metformin-empagliflozin, Synjardy, metformin-glipizide, metformin-glyburide, Glucovance, metformin-linagliptin, Jentadueto, metformin-pioglitazone, Actoplus, Actoplus Met, Actoplus Met XR, metformin-repaglinide, PrandiMet, metformin-rosiglitazone, Avandamet, metformin-saxagliptin, Kombiglyze XR, metformin-sitagliptin, Janumet, or Janumet XR; bromocriptine such as but not limited to Parlodel; alogliptin such as but not limited to Nesina; alogliptin-pioglitazone such as but not limited to Oseni; linagliptin such as but not limited to Tradjenta, linagliptin-empagliflozin such as but not limited to Glyzami; saxagliptin such as but not limited to Onglyza; sitagliptin such as but not limited to Januvia; sitagliptin and simvastatin such as but not limited to Juvisync; albiglutide such as but not limited to Tanzeum; dulaglutide such as but not limited to Trulicity; exenatide such as but not limited to Byetta; exenatide extended-release such as but not limited to Bydureon; liraglutide such as but not limited to Victoza; nateglinide such as but not limited to Starlix; repaglinide such as but not limited to Prandin; dapagliflozin such as but not limited to Farxiga; canaglifoxin such as but not limited to Invokana; empaglifozin such as but not limited to Jardiance; empagliflozin-linagliptin such as but not limited to Glyxambi; glimepiride such as but not limited to Amaryl; glimepiride-pioglitazone such as but not limited to Duetact; glimepiride-rosiglitazone such as but not limited to Avandaryl; gliclazide, glipizide such as but not limited to Glucotrol; glyburide such as but not limited to DiaBeta, Glynase, or Micronase; chlorpropamide such as but not limited to Diabinese; tolazamide such as but not limited to Tolinase; tolbutamide such as but not limited to Orinase or TolTab; rosiglitazone such as but not limited to Avandia; or pioglitazone such as but not limited to Actos. In some embodiments, the treatment regimen includes administration of one or more pharmaceutical drugs, each administered separately to a subject; behavioral modification such as dietary changes and increased daily exercise; or surgery such as bariatric surgery.

Embodiments of the invention can be a component of a kit. In one embodiment, the kit includes (a) a container that contains the Br⁻ or Br-comprising composition, and optionally (b) informational material. The informational material can be descriptive, instructional, marketing or other material that relates to the methods described herein and/or the use of the agents for therapeutic benefit. In an embodiment, the kit includes also includes a second agent, such as a pharmaceutical drug as described herein. For example, the kit includes a first container that contains the Br⁻ composition, and a second container that includes the pharmaceutical drug. As another example, the kit includes a container that contains the Br′ composition in combination with the pharmaceutical drug.

### Use of the Invention in Cancer

Patients with cancer receiving chemotherapeutic or radiation treatment experience an elevated risk of developing cardiovascular disease due to the cancer treatment. Non-limiting examples of the forms of these diseases that can be triggered by cancer treatments comprise Q-T prolongation, hypertension, heart failure, cardiomyopathy, thromboembolism, edema, ischemia, atrial fibrillation, cardiac shock, bradycardia, and kidney injury. Non-limiting examples of specific chemotherapeutics that can cause these forms of disease comprise arsenic trioxide, bevacizumab, bortezomib, cisplatin, anthracyclines, doxorubicin, fluorouracil 5-FU, imatinib, interleukin alpha-2b, lapatinib, lenalidomide, melphalan, mitomycin, mitoantrone, pazopanib, sorafenib, sunitinib, thalidomide, trastuzumab, carmustine, and methotrexate.

Natriuretic peptides can inhibit metastasis, or spreading, of cancerous cells. Increased concentrations of BNP and ANP are associated with decreased incidence of metastasis (Masago et al., Oncol. Lett., 2(2):253-247, 2011; Vesely, Anticancer Res., 34(4):1459-1466, 2014; Serafino and Pierimarchi, Curr. Med. Chem., 21(21):2401-2409, 2014). Moreover, disruptions in collagen IV have also been associated with metastasis (Burnier et al., Oncogene, 30:3766-3783, 2011). Embodiments of the invention can be administered to a subject with cancer in order to prevent metastasis.

Embodiments of the invention can administer therapeutically effective amounts of Br⁻ to a subject receiving cancer treatment. Such practice of the invention can prevent or treat cardiovascular disease in a subject with cancer or who has received a cancer treatment. The invention can assist in preventing the development of a form of cardiovascular disease, kidney disease, or a combination thereof in a subject with cancer. Alternatively, the invention can assist in the recovery of the subject who has been treated with one or more cancer treatment regimens. The subject can be receiving radiation treatments, surgery, chemotherapy, or a combination thereof. In some embodiments, the invention can be administered to a subject before, after, or during an individual cancer treatment. The cancer treatment can be chemotherapy, surgery, radiation, or a combination thereof. In some embodiments, the invention can be administered to a subject before the subject is placed onto a cancer treatment regimen. For example, after a subject is diagnosed with cancer yet before the subject begins a set of cancer treatments or surgery, the subject can be administered one or more embodiments of the invention in preparation for the upcoming cancer treatments or surgery. In some embodiments, the invention can be administered to a subject after the subject has completed a cancer treatment regimen. For example, a subject who has completed a specified chemotherapy, radiation, or surgical treatment regimen may still be administered embodiments of the invention in order to assist the recovery of the subject. In another example, a subject can be administered embodiments of the invention days, weeks, months, or years after completing a cancer treatment regimen in order to prevent the development of cardiovascular disease, kidney disease, or a combination thereof. Embodiments of the invention can be similarly administered to subjects who do not complete a cancer treatment regimen, or who change from one cancer treatment regimen to another.

In some embodiments, the invention can comprise an effective amount of Br⁻ and a chemotherapeutic agent, for co-administration a subject. Non-limiting examples of chemotherapy and radiotherapy comprises methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, x-rays, gamma rays, and charged particles.

### Applications of the Invention

Embodiments of the invention are amenable for any application where current Br-deficient medical saline solutions are used. Non-limiting examples of these comprise administration of embodiments to a subject via injection, topical, or oral route; applying embodiments of the invention to a medical device; and combining embodiments with pharmaceutical drugs.

### Administration of Pharmaceutical Drugs via the Invention

Br-deficient saline is frequently used to dilute and administer pharmaceutical drugs to subjects. However, by perturbing the halide balance in a subject, as described herein, Br-deficient 0.9% saline can interfere with the therapeutic activity of the drug. Embodiments of the invention provide an improved, physiologically-relevant solution for diluting and administering drugs to a subject, wherein the solution maintains or restores the halide balance in a subject, thereby reducing the risk of inducing a Br-deficient saline-associated side effect.

Some drugs are known to deplete Br⁻ from subjects, comprising but not limited to diuretic agents and chemotherapy drugs. Non-limiting examples of such drugs are described herein. Subjects receiving Br-depleting drugs can experience low Br⁻ levels, which by definition would be an altered halide balance. In clinical situations like these, administration of Br-deficient 0.9% saline can cause unwanted side effects, due to the pre-conditioned halide imbalance in the subject. Embodiments as described herein can be useful for maintaining or restoring physiologically relevant amounts of Br⁻ and Cl⁻ in these subjects as they undergo treatments for disease.

In some embodiments, compositions as described herein can be combined with a pharmaceutical drug, drug ingredient, or medical device. In some embodiments, compositions as described herein can be used to administer the drug to a subject. Many types of drugs can be administered to subjects using embodiments as described herein. Non-limiting examples of drug types comprise small molecules, proteins, antibodies, vaccines, and biologics. Embodiments of the invention can be combined with natural products for co-administration to a subject. Non-limiting examples of natural products comprise nutraceuticals, dietary supplements, herbal medicines, minerals, and foods.

Embodiments of the invention can be used to treat many different types of diseases and medical conditions in a subject. In some embodiments, compositions of the invention are combined with a drug or device to treat or care for a disease or medical condition in a subject. Non-limiting examples of diseases and medical conditions that can be treated by embodiments as described herein comprise cancer, eye disease or injury, ear disease, kidney disease or injury, cardiovascular disease, gastrointestinal disorder or disease, or an endocrinopathy, such as diabetes, metabolic syndrome, or obesity, cystic fibrosis, sepsis, fluid loss, infection, neurodegenerative disease a degenerative disease, a blood disorder, diabetes, a wound, a skin irritation, inflammation, an injury, a medical condition requiring dialysis treatment, or a combination thereor. Non-limiting symptoms of these diseases, which may be treated by embodiments of the invention, comprise vascular disease; chronic kidney disease; end stage renal disease; glomerular disease; tubular disease; kidney injury; acute kidney injury; sepsis-induced kidney injury; drug-induced kidney injury; hypovolemia-induced kidney injury; ischemic kidney injury; fibrosis; vascular disease; hypertension; salt-sensitive hypertension; heart attack; heart failure; cardiac remodeling; cardiac fibrosis; myocardial fibrosis; atherosclerosis; stroke; arterial stiffening; vascular wall thickening; thickening of the peritoneal membrane; dyslipidemia; blood clot; anemia; an acid-base imbalance; hypercholemia; infection; sepsis; thrombosis; coronary artery disease; ischemic heart disease; peripheral artery disease; heartburn; indigestion; nausea; vomiting; peptic ulcers; abdominal pain; belching; bloating; flatulence; gallstones; constipation; diarrhea; hemorrhoids; rectal problems; vision loss; central vision loss; peripheral vision loss; blindness; blurry vision; cloudy vision; distorted vision; pink eye; eye infection; bulging eyes; bumps on the eye or eyelid; burning sensation in eyes; crusty eyelids; altered depth perception; discharge from eyes; double vision; dry eyes or dry eyelids; the sensation of a foreign body in the eye; gritty sensation in the eye; corneal lesion; light flashes; spots on eye lid; yellow eyes; fever; chills; decreased urination; tachycardia; tachypnea; hyperventilation; presence of bacteria or fungi in the bloodstream; unexplained weight loss; extreme fatigue; pain; skin changes such as change in the color or size or shape of skin mole; chronic sores; one or more lumps in a tissue such as a lump in a breast or lymph node; bleeding or discharge; chronic cough; hearing loss; tinnitus; congestion; infection; type 1 diabetes; type 2 diabetes; type 3 diabetes; gestational diabetes; juvenile diabetes; latent autoimmune diabetes of adulthood; maturity onset diabetes of the young; insulin resistance; hyperglycemia; steroid-induced diabetes; brittle diabetes; diabetes insipidus; diabetes mellitus; hemoglobin A1C levels between above 5.7% and 6.4%; hemoglobin A1c levels above 6.4%; syndrome x; an individual displaying at least three of the following metabolic risk factors: waist size greater than 40 inches if male subject or greater than 35 inches if female subject, blood tryglyceride levels of at least about 150 mg/dl or higher or currently using a cholesterol medication, blood high-density lipoprotein levels lower than about 40 mg/dl if male subject or lower than about 50 mg/dl if female subject or currently using a cholesterol medication, blood pressure above about 135/85 mm Hg (systolic over diastolic) or using a high blood pressure medication, and fasting blood glucose levels of about 100 mg/dl or higher; or a combination thereof.

Some embodiments of the invention can be administered to a subject after a surgical operation. Some embodiments of the invention can be administered to a subject who is suffering from a side effect of a previously administered pharmaceutical drug. Some embodiments of the invention can be administered as a second-line, third-line, or later prescribed therapy. For example, an embodiment of the invention can be administered to a subject who has developed hyperchloremia due to a previous intravenous administration of Br-deficient 0.9% NaCl saline. In some embodiments, the drug or device is used to promote tissue regeneration. In some embodiments, the drug is chemically defined as a protein, recombinant protein, cellular therapeutic, antibody, biologic, molecule, liposome, lipid, deoxyribonucleic acid, ribonucleic acid, or small molecule.

Many types of drugs can be administered via injection to subjects in combination with embodiments of the invention as described herein. Non-limiting examples of such drugs that can be combined with compositions of the invention described herein comprise a blood pressure controlling agent, an angiotensin-converting enzyme inhibitor, an angiotensin receptor blocker, a beta blocking agent, an alpha blocking agent, an anti-arrhythmic agent, a blood thinner, an alpha agonist, a sodium channel blocking agent, a calcium channel blocking agent, an anti-platelet agent, an anti-hyperlipidemic agent, a statin, a nonsteroidal anti-inflammatory drug, a loop diuretic, a thiazide diuretic, a potassium-sparing diuretic, a vasodilator, a renin inhibitor, dopamine, a dopamine receptor agonist, a thrombolytic agent, erythropoietin, an erythropoietic stimulating agent, a vitamin, a vitamin analogue, a drug used in the management of ESRD, an anti-infective agents, an antibiotic, an antifungal, a cancer chemotherapeutic agent, a steroid, an injectable drug, a topical drug, an eye drop or ointment, a drying agent for the ear, an anti-inflammatory drug, a prostaglandin analogue, a carbonic anhydrase inhibitor, an adenergic agonist, an anti-allergy drug, an angiogenesis inhibitor drug, an 5-aminosalicylates, an antacid, an anti-diarrheal, a digestive enzyme, a chloride channel activator, a guanylate cyclase-C agonist, a peripheral opioid receptor agonist, a peripheral opioid receptor antagonist, a gallstone solubilizing agent, a gastrointestinal stimulant, a Helicobacter pylori eradication agent, a histamine-2(H2) blocker, an antiacid, a laxative, an osmotic laxative, a polyethylene glycol osmotic, a gastrointestinal simulant, a promotility agent, a stool softener, a bulk-forming fiber, a proton pump inhibitor, a flu shot, an infectious-disease vaccine, a cancer vaccine, a insulin agent, an amylinomimetic agent, an alpha-glucosidase inhibitor, a biguanide, a dopamine agonist, a glucagon-like peptide, a meglitinide agent, a sodium glucose transporter 2 inhibitor, a sulfonylurea agent, a thiazolidinedione agent, and a dipeptidyl peptidase-4 inhibitor, or a combination thereof. Non-limiting examples of drugs that may be combined with embodiments of the invention comprise captopril, enalapril, fosinopil, lisinopril, perindopril, quinapril, trandolapril, benazepril, ramipril, azilsartan, candesartan, telmisartan, fimasartan eprosartan, irbesartan, losartan, olmesartan, valsartan, doxazosin, phentolamine, indoramin, phenoxybenzamine, tolazoline, bucindolol, carvedilol, labetalol, tamsulosin, terazosin, prazosin, alfuzosin, timolol, betaxolol, propranolol, atenolol, nadolol, nebivolol, oxprenolol, pindolol, propranolol, metoprolol, sodium nitroprusside, hydralazine, adenosine, sildenafil, vardenafil, tadalafil, prostacyclin, nitric oxide, amiodarone, mexiletine, disopryamide, propafenone, diltiazem, dihydropyridines, amlodipine, cilnidipine, felodipine, isradipine, nimodipine, lercanidipine, levamlodipine, nicardipine, nitrendipine, nifedipine, verapamil, spironolactone, bumetanide, ethacrynic acid, epitizide, metolazone, amiloride, triamterene, torsemide, furosemide, indapamide, triamterene, hydrochlorothiazide, chlorothiazide, bendroflumethiazide, chlorthalidone, celecoxib, meloxicam, ibuprofen, naproxen, diclofenac, aspirin, dipyridamole, clopidogrel, cilostazol, ticlopidine, lovastatin, niacin, simvastatin, ezetimibe, warfarin, carperitide (recombinant ANP), nesiritide (recombinant BNP), tinzaparin, enoxaparin, heparin, atorvastatin, fluvastatin, pravastatin, rosuvastatin, aliskiren, alteplase, anistreplase, reteplase, tenecteplase, streptokinase, tissue plasminogen activator, urokinase, recombinant human erythropoietin, epoetin alpha, epoetin beta, darbepoetin alpha, methoxy polyethylene glycol-epoetin beta, Epo, Procrit®, Epogen®, Aranesp, Mircera, vitamin D, rocaltrol, calcitriol, zemplar®, hectorol, doxercalciferol, carnitor, levocarntine, lepiridun, reteplase, alteplase, peginesatide, iron, sodium ferric gluconate, vitamin B12, Darbepoetin, midazolam hydrochloride, diazepam, calcium gluconate, calcitonin, deferoxamine, doxercalciferol, ibandronate, pamidronate, paricalcitol, methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, penicillin, amoxicillin, cephalexin, erythromycin, clarithromycin, azithromycin, ciprofloxacin, levofloxacin, ofloxacin, sulfamethoxazole, trimethoprim, fosfomycin, nitrofurantoin, ceftriaxone, clavulanate, clindamycin, doxycycline, tetracycline, clotrimazole, econazole nitrate, miconazole, terbinafine, fluconazole, ketoconazole, and amphotericin. Non-limiting examples of chemotherapy and radiotherapy comprises methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, x-rays, gamma rays, charged particles, and combinations thereof. In some embodiments, the drug comprises regular insulin such as but not limited to Humulin or Novolin, insulin aspart such as but not limited to Novolog or FlexPen; insulin glulisine such as but not limited to Apidra; insulin lispro such as but not limited to Humalog; insulin isophane such as but not limited to Humulin N or Novolin N; insulin degludec such as but not limited to Tresiba; insulin detemir such as but not limited to Levemir; insulin glargine such as but not limited to Lantus; insulin glargine such as but not limited to Toujeo; a combination insulin drug such as but not limited to insulin aspart protamine-insulin aspart, insulin lispro protamine-insulin lispro, human isophane insulin-human insulin regular, insulin dedludec-insulin aspart, NovoLog Mix 70/30, Humalog Mix 75/25, Humalog Mix 50/50, Humalin 70/30, Novolin 70/30, or Ryzodeg; pramlintide such as but not limited to SymlinPen; acarbose such as but not limited to Precose; miglitol such as but not limited to Glyset; metformin such as but not limited to Glucophage, Metformin Hydrochloride ER, Glumetza, Riomet, or Fortamet; a metformin-containing drug such as but not limited to metformin-alogliptin, Kazano, metformin-canagliflozin, Invokamet, metformin-dapagliflozin, Xigduo XR, metformin-empagliflozin, Synjardy, metformin-glipizide, metformin-glyburide, Glucovance, metformin-linagliptin, Jentadueto, metformin-pioglitazone, Actoplus, Actoplus Met, Actoplus Met XR, metformin-repaglinide, PrandiMet, metformin-rosiglitazone, Avandamet, metformin-saxagliptin, Kombiglyze XR, metformin-sitagliptin, Janumet, or Janumet XR; bromocriptine such as but not limited to Parlodel; alogliptin such as but not limited to Nesina; alogliptin-pioglitazone such as but not limited to Oseni; linagliptin such as but not limited to Tradjenta, linagliptin-empagliflozin such as but not limited to Glyzami; saxagliptin such as but not limited to Onglyza; sitagliptin such as but not limited to Januvia; sitagliptin and simvastatin such as but not limited to Juvisync; albiglutide such as but not limited to Tanzeum; dulaglutide such as but not limited to Trulicity; exenatide such as but not limited to Byetta; exenatide extended-release such as but not limited to Bydureon; liraglutide such as but not limited to Victoza; nateglinide such as but not limited to Starlix; repaglinide such as but not limited to Prandin; dapagliflozin such as but not limited to Farxiga; canaglifoxin such as but not limited to Invokana; empaglifozin such as but not limited to Jardiance; empagliflozin-linagliptin such as but not limited to Glyxambi; glimepiride such as but not limited to Amaryl; glimepiride-pioglitazone such as but not limited to Duetact; glimepiride-rosiglitazone such as but not limited to Avandaryl; gliclazide, glipizide such as but not limited to Glucotrol; glyburide such as but not limited to DiaBeta, Glynase, or Micronase; chlorpropamide such as but not limited to Diabinese; tolazamide such as but not limited to Tolinase; tolbutamide such as but not limited to Orinase or TolTab; rosiglitazone such as but not limited to Avandia; pioglitazone such as but not limited to Actos; and combinations thereof. In some embodiments, the treatment regimen includes administration of one or more pharmaceutical drugs, each administered separately to a subject; behavioral modification such as dietary changes and increased daily exercise; or surgery such as bariatric surgery. In some embodiments, a drug and a composition of the invention can be co-administered to a subject in a single injection. In other embodiments, a drug and a composition of the invention can be separately administered to a subject. For example, a composition of the invention can be injected into a subject and a drug can be separately administered to the same subject. In embodiments, the drug can be administered to a subject before a composition as described herein is administered to the subject. In other embodiments, the drug can be administered to a subject after a composition as described herein is administered to the subject. In still other embodiments the drug and the composition as described herein are administered at about the same time as each other.

Some embodiments of the invention can be administered to a subject with an endocrinopathy (e.g., diabetes or metabolic syndrome). For example, subjects with diabetes can develop metabolic acidosis if too much Br-deficient 0.9% NaCl is administered. In this case, administration of an embodiment of the invention, comprising a physiologically balanced amount Cl⁻ and Br⁻, can reduce the risk of acidosis in subject. In some embodiments, a composition of the invention is co-administered with a drug used in the management of diabetes or metabolic syndrome. In some embodiments, a composition of the invention and a medical device are both administered to (or used on) a subject with diabetes. For example, compositions of the invention and bandages can both be used in providing medical care for a diabetic ulcer.

In some embodiments, compositions of the invention are used for the manufacturing of a drug or drug ingredient. For example, embodiments of the invention can be used to solubilize or suspend an active pharmaceutical ingredient of a drug as described herein. As another example, certain drugs that are suitable for injection, many of which are described herein, can use embodiments of the invention as the liquid vehicle for the drug. Non-limiting examples of such injection drugs comprise drugs used in the management of ESRD, cancer chemotherapies, and eye drugs.

Some embodiments of the invention can be used for manufacturing a medical device. Non-limiting examples of the medical devices comprise a surgical device, a syringe, a needle, a suture, a contact lens, a medical device used in dialysis, a dialysis machine, a port, a fistula, a catheter, a graft, a machine used in the medical care of a subject, a component of a machine used in the medical care of a subject, a stent, a plug, a patch, an injection filler, a powder, a bandage, a gauze, a negative pressure device, a hydrophobic dressing, a hemostatic agent, an absorbent, a hydrating agent, an osmotic agent, a preservative, a wash, a lotion, a cream, a gel, a paste, an ointment, a rinse, an eye drop, an ear drop, a spray, a nasal spray, a matrix, a scaffold, an acellular scaffold, a collagen-based device, a camera, a pill camera, a tube, a connector, a needlefree connector, a closed system, transfer device, a hemodynamic monitoring system, a bag, a balloon, an intravenous line, a central line, an arterial line, or a combination thereof. For example, embodiments of the invention can be added to a bandage during packaging, in order to moisten as well as maintain adequate moisture in the bandage prior to its use on a subject. As another example, embodiments of the invention can be used to add an effective amount of Br⁻ to a dialysate. As still another example, embodiments of the invention can be used to add effective amounts of Br⁻ and Mg²⁺ to a dialysate.

Some embodiments can be contacted with an organ, tissue, or tissue product. Non-limiting examples of such organs, tissues, or tissue products comprise harvested tissues, engineered tissues, cultured tissues, human tissues, animal tissues, autologous tissues, bone, ligaments, tendons, vascularized organs, lung, kidney, heart, heart tissue, liver, pancreas, corneas, blood, stem cells, progenitor cells, skin, dura mater, oocytes, semen, and combinations thereof. Some embodiments can be used to cleanse, wash, moisten, or lubricate the organ, tissue, or tissue product. Some embodiments can be used to harvest, culture, engineer, transport, store, or administer an organ, a tissue, or a tissue product to a subject. Some embodiments can be co-administered with a medical device to a subject. For example, a wound in a subject can be treated with a moist bandage where an embodiment of the invention is used to moisten the bandage. As another example, an embodiment of the invention can be used to wash or hydrate a wound in a subject prior to treating the wound with a collagen-based plug, patch, powder, or bandage. As yet another example, an embodiment of the invention and an intravascular stent can be co-administered to a subject with vascular disease. As still another example, an embodiment of the invention and an intravascular balloon can be co-administered to a subject who is undergoing a surgical procedure. Non-limiting examples of the medical devices comprise a surgical device, a syringe, a needle, a suture, a contact lens, a medical device used in dialysis, a dialysis machine, a port, a fistula, a catheter, a graft, a machine used in the medical care of a subject, a component of a machine used in the medical care of a subject, a stent, a plug, a patch, an injection filler, a powder, a bandage, a gauze, a negative pressure device, a hydrophobic dressing, a hemostatic agent, an absorbent, a hydrating agent, an osmotic agent, a preservative, a wash, a lotion, a cream, a gel, a paste, an ointment, a rinse, an eye drop, an ear drop, a spray, a nasal spray, a matrix, a scaffold, an acellular scaffold, a collagen-based device, a camera, a pill camera, a tube, a connector, a needlefree connector, a closed system, transfer device, a hemodynamic monitoring system, a bag, a balloon, an intravenous line, a central line, an arterial line, or a combination thereof.

In some embodiments, compositions of the invention are administered to a subject with cancer. In some embodiments, the cancer comprises solid tumor; metastatic tumor; epithelial cancer; circulating cancer cells; eye cancer; kidney cancer; childhood cancer; brain cancer; spinal cord tumor; liver cancer; bone cancer; colorectal cancer; stomach cancer; small intestine cancer; prostate cancer; breast cancer; skin cancer; basal cell cancer; squamous cell skin cancer; melanoma; multiple myeloma; lung cancer; lung tumor; small cell lung cancer; non-small cell lung cancer; blood cancer; leukemia; lymphoma; Hodgkin's lymphoma; non-Hodgkin's lymphoma; bladder cancer; oral cancer; oropharyngeal cancer; pancreatic cancer; thyroid cancer; thymus cancer; uterine cancer; uterine sarcoma; cervical cancer; ovarian cancer; testicular cancer; Wilms tumor; acute lymphocytic leukemia, chronic lymphocyte leukemia; acute myeloid leukemia; chronic myeloid leukemia; chronic myelomonocytic leukemia; adrenal cancer; anal cancer; bile duct cancer; endometrial cancer; esophagus cancer; a Ewing tumor; gallbladder cancer; gastrointestinal tumor; Kaposi sarcoma; laryngeal cancer; hypopharyngeal cancer; malignant mesothelioma; Merkel cell skin cancer; myelodysplastic syndrome; cancer of the nasal cavity; paranasal sinus cancer; nasopharyngeal cancer; neuroblastoma; osteosarcoma; penile cancer; pituitary tumor; retinoblastoma; rhabdomyosarcoma; salivary gland cancer; soft tissue sarcoma; vaginal cancer; vulvar cancer, or a combination thereof. In some embodiments, the cancerous tissue is found in the bone, bone marrow, blood, lymph, lymph nodes, immune cells, breast, skin, prostate, ovary, cervix, uterus, vagina, penis, testicles, bladder, intestine, colorectal region, pancreas, bile duct, stomach, gastrointestinal tract, thyroid, soft tissue, lung, liver, kidney, adrenal glands, brain, spinal region, central nervous system, peripheral nervous system, eye, gums, lips, mouth, salivary gland, tonsils, jaw, esophagus, throat, sinus, nasopharyngeal region, head and neck region, heart, or a combination thereof. In some embodiments, compositions of the invention are administered to a subject with cancer through a medical tube, such as but not limited to a central line. In some embodiments, compositions of the invention are used to flush a medical line or preserve an injection site in a subject receiving cancer treatments. In some embodiments, compositions of the invention are combined with a cancer drug, such as but not limited to a cancer chemotherapeutic drugs, and administered to a subject. Non-limiting examples of cancer chemotherapeutics comprise methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, carfilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, and combinations thereof.

Some embodiments of the invention can be used to treat or prevent a side effect of cancer. Non-limiting examples of side effects of cancer comprise mouth sores, oral thrush, changes in blood pressure, thrombosis, electrocardiographic changes, arrhythmia, myocarditis, pericarditis, myocardial infarction, cardiomyopathy, cardiac failure, congestive heart failure, kidney injury, hair loss, nausea, vomiting, diarrhea, neutropenia, thrombocytopenia headaches, nerve pain, weight loss, anemia, infection, fatigue, loss of or reduced fertility, and combinations thereof. In some embodiments, a composition of the invention is administered to a subject with cancer. For example, embodiments of the invention can be administered as an oral rinse to a subject with cancer to prevent or treat mouth sores. In some embodiments, a composition of the invention is co-administered with a drug to a subject with cancer. In some embodiments, a composition of the invention is combined with a medical device for administration to a subject with cancer.

### Injection Applications

Biologically, Br⁻ can be found in many sites where Br-deficient 0.9% saline is regularly injected, such as but not limited to the bloodstream, muscle, and eye. Administration of Br-deficient 0.9% saline can deplete Br⁻ from these locations, causing a halide imbalance in a subject. In many instances, the Br-deficient 0.9% saline solution is being administered to a subject to treat a primary health concern, causing an unwanted secondary health concern stemming from the halide imbalance caused by the Br-deficient solution. Addressing this unwanted side effect of the Br-deficient solution, embodiments of the invention provide physiologically-balanced halide solutions that better mimic the physiologic balance of Br⁻ and Cl⁻ in a healthy individual, resulting in less of a “treatment burden” on the subject compared to Br-deficient 0.9% saline. This allows the subject's body to direct its physiologic resources towards healing the primary disease, health concern or injury, rather than forcing the subject's body to exert its energy towards recovering from the secondary health concern that stems from administration of Br-deficient 0.9% saline administration.

In humans, Br⁻ and Cl⁻ are primarily found in the extracellular space. Consequently, embodiments of the invention that are injected into a subject can provide a rapid means of restoring a physiologic balance of Br⁻ and Cl⁻ in a subject who is in need thereof, such as a subject as described herein.

Embodiments of the invention can be injected into the bloodstream, skin, tissue, peritoneal cavity, orbital socket, heart, muscle, bone marrow, epidural space, spinal theca, joint, bladder, genitals, or any other organ of a subject. In some embodiments, the invention is injected through a needle into a subject. For example, embodiments can be injected from a syringe into a subject using a needle. For example, the invention can be an ingredient in a drug that is administered through a syringe and needle into an intramuscular injection site. In this example, the invention can better mimic the halide balance in tissues and thereby reduce the potential for side effects at the drug injection site. In some embodiments, the invention is injected through a tube. For example, the invention can be injected into a subject through a catheter. In some embodiments, the invention is injected through a tube into the blood of a subject. For example, for subjects that are receiving extracorporeal blood purification therapy, such as hemodialysis, embodiments can be injected through a tube into the subject's bloodstream while the blood is outside the body. As another example, some subjects can be administered embodiments through a central line, where the embodiment is stored in a bag with a connected tube that transfers fluid from the bag to the subject. In this case, the tube can inject the embodiment through a connector that allows fluid to be directly injected into a subject's bloodstream. Non-limiting examples of connectors include vascular assess ports, implanted ports, intravenous lines, peripheral intravenous lines, and dialysis assess ports. As yet another example, the invention can be an ingredient in a vaccine that is administered via intramuscular injection. In this example, the invention can better mimic the halide balance in tissues and thereby reduce the potential for side effects of the vaccine.

Embodiments of the invention can be used for eye injections. The anatomy of the eye is very complex, comprising multiple layers of tissue that separate the eye surface (e.g. the external surface of the cornea) and the back of the eye or the retina. As a result, the administration of drugs to the back of the eye typically requires an injection to the eye, since topical administration is unlikely to deliver sufficient amounts of a drug compound to the back of the eye. For example, Eylea®, an FDA-approved drug for treating macular degeneration, is administered via eye injection. As an injection, this drug is delivered as a liquid. Saline solutions can provide convenient liquids for such injectable eye drugs.

The eye contains a large amount of BC, and administration of Br-deficient 0.9% NaCl can perturb the halide balance in the eye. The invention is formulated to better mimic the halide balance in a healthy subject, thereby enhancing the potential for the eye to positively respond to the drug. Some embodiments of the invention can be administered to an eye via non-injection routes as described herein. For example, a non-injection route comprises topical administration to an eye, such as a wash, drop, ointment, or cream.

In some embodiments, compositions of the invention are administered to a subject with eye disease. In some embodiments, the eye disease comprises a retinal disease, a neurodegenerative eye disease, macular degeneration, glaucoma, dry eye disease, or keratoconus. In some embodiments, compositions of the invention are administered by injection or by topical administration. In some embodiments, compositions of the invention are administered to a subject using a needle, syringe, tube, dropper, bottle, contact lens, or combination thereof. In some embodiments, compositions of the invention are administered to a subject who before, during, or after a medical operation is performed in one or both of the subject's eyes, orbital sockets, or a combination thereof. For example, compositions of the invention may be administered to a subject who receives laser assisted in situ keratomileusis (LASIK). As another example, compositions of the invention may be administered to a subject who receives cataract surgery.

Embodiments of the invention can be injected into the bloodstream. Br-deficient 0.9% saline is frequently injected into a bloodstream such as but not limited to during flushing of a central line after administration of a cancer chemotherapy or during the treatment of sepsis. In the case of subjects with cancer, administration of chemotherapy can deplete a subject of circulating Br⁻ (Weber et al., 1984); if Br-deficient 0.9% saline is also administered, it may further deplete the individual of Br³¹ . In the case of sepsis treatment, subjects can require large volumes of saline, which can perturb the physiologic halide balance in the subject. Indeed, administration of Br-deficient 0.9% saline is associated with poorer treatment outcomes when compared to administration of other Br-deficient saline formulations (Yunos et al., 2010). In some instances, such Br-deficient saline formulations also have lower amounts of other non-Br ingredients, non-limiting examples of which comprise Cl, lactate, starch, potassium, calcium, or a combination thereof. Thus, injection applications of the invention can be useful in preventing perturbation of the halide balance in a subject.

In some embodiments, compositions of the invention comprise an amount between about 30 and about 1 mM bromide ions (Br⁻), an amount between about 17.1 mM and about 200 mM chloride ions (Cl⁻), and a therapeutically effective amount of at least one of lactate, calcium, magnesium, bicarbonate, potassium, sodium, hydrogen phosphate, chloride, dextrose, or a combination thereof. For example, compositions of the invention can comprise Br⁻, Cl⁻, and at least one of the following:
About 0-about 35 mEq/L lactate,
About 1-about 5 mEq/L calcium,
About 0.5-about 3 mEq/L magnesium
About 22-about 32 mEq/L bicarbonate (HCO₃⁻),
About 0-about 4 mEq/L potassium,
About 140 mEq/L sodium,
About 0-about 1 mEq/L hydrogen phosphate (HPO₄²⁻),
About 100-about 130 mEq/L chloride,
About 0-about 100 mEq/L dextrose,
or a combination thereof.

An embodiment of the invention as described herein comprises an intravenous saline that comprises NaBr and NaCl. For example, an embodiment can comprise 100 μM NaBr and 100 mM NaCl. Manufacturing of this embodiment can proceed in two basic steps: first, manufacturing a reagent embodiment of the invention that comprises a Br-to-Cl molar ratio of 0.001, or 0.1% Br⁻ to Cl⁻, and second, manufacturing the injection saline. This protocol is appropriate for manufacturing up to 100 L of the saline. Other manufacturing protocols are also suitable for manufacturing embodiments of the invention, with a non-limiting example comprising adding an appropriate amount of Br⁻ to a Br-deficient saline solution. A more detailed description of the above-described two-step manufacturing process is described below.

For manufacturing the reagent, approximately 584.4 g of NaCl (USP) and approximately 1.0289 g of NaBr (USP) are dissolved in water, filtered, and dried to a powder, yielding a sodium-based reagent containing 0.001 Br-to-Cl ratio. For quality purposes, 90 mg of the reagent is dissolved in 7 ml of water, the volume adjusted to 10 ml, and the sample analyzed by inductively coupled plasma mass spectrometry to validate the actual Br-to-Cl ratio. For production of 10 L batches, 90 g of the reagent is dissolved in 7 L of water and the volume adjusted to 10 L. The solution can be packaged in 1 L bags for intravenous administration. The product can be sterilized according to any sterilization method practiced by the skilled artisan, such as aseptic techniques or terminal sterilization. The product is useful for injection into the bloodstream of a subject, such as during continuous renal replacement therapy, the treatment of sepsis, or when cleaning or flushing a central line or catheter in a subject. The product can also be used to clean a dialysis access port or to wash a bleeding tissue during surgery.

### Use of Invention in Acute Kidney Injury

When acute kidney injury (AKI) occurs in a subject, it develops rapidly, such as within 48 hours, and can lead to complete loss of kidney function. Subjects with AKI can require a type of dialysis treatment called continuous renal replacement therapy (CRRT). Risk factors for AKI include sepsis, intravenous contrast agents, certain drugs including chemotherapies, and surgical operations. Considering the rapid development period for AKI, hospitalized subjects that are at risk of developing AKI require monitoring of kidney function in order to detect early stages of AKI.

The importance of Br⁻ in kidney physiology has been unappreciated, resulting in a lack of any treatments that either promote halide balance or provide a therapeutically effective amount of Br⁻ to subjects with AKI. Embodiments as described herein address this unmet need, comprising embodiments that comprise physiologically-balanced amounts of Br⁻ to preserve and/or improve kidney function in a subject with AKI.

Br-deficient medical saline, which does not comprise physiologically relevant amount of Br⁻, is frequently administered to subjects at risk of developing AKI, such as but not limited to subjects with sepsis. In this case, Br-deficient intravenous saline can be administered to subjects with sepsis in order to maintain adequate blood pressure, such as during septic shock. The administration of Br-deficient 0.9% saline can elevate the risk of hyperchloremia and/or acidosis in the subject (Yunos et al., 2010). Embodiments of the invention, which comprise physiologically relevant amount of Br⁻, can be intravenously administered to subjects with sepsis in order prevent the development of AKI, maintain adequate blood pressure in the subject, prevent organ failure in the subject, assist the subject in maintaining physiologically balanced amounts of Br⁻ and Cl⁻, or a combination thereof.

Br-deficient medical saline is frequently administered to subjects with AKI. For example, replacement fluids can be administered during the course of CRRT, in order to maintain adequate blood volume in the subject. Br-deficient saline is frequently used as the replacement fluid, yet this presents the risk that the subject will develop an electrolyte imbalance such as hyperchloremia or acidosis. Embodiments of the invention can be used as CRRT replacement fluids to maintain volume with less risk of developing an electrolyte imbalance. Embodiments of the invention can also be used in the manufacture of CRRT dialysates that maintain adequate Br⁻ levels in the subject.

Some embodiments of the invention can be injected through a needle and syringe to a subject on CRRT, with non-limiting examples of the injection method comprising intramuscular, intradermal, subcutaneous, intravenous, intraosseous, intraperitoneal, intracardiac, intrathecal, epidural, intraarticular, intracavernous, and intravitreal. In this example, the embodiments can be manufactured for distributed in a vial that requires a medical professional to draw up an appropriate amount of the composition into a syringe for administration. This conveniently allows the medical professional to adjust the administered dose to the individual needs of the subject. Alternatively, the invention can be distributed in a pre-filled syringe to reduce the number of actions required of the medical professional.

In another embodiment, compositions of the invention are added to a replacement fluid that is used during CRRT. In this case, the invention can be injected or infused into the subject's bloodstream before or after the blood is contacted with a filter. In other words, the compositions can be administered “pre-filter” or “post-filter.” In some embodiments, compositions are administered to the bloodstream while the blood is outside the body, also termed extracorporeal administration. In some embodiments, compositions are distributed in a bag that is compatible with intravenous administration.

### Use of Invention as a Dialysis Fluid or Dialysate

Subject with end stage renal disease (ESRD) who are being treated with maintenance dialysis have a high risk of developing cardiovascular disease as well as increased risk of an early death (2015 USRDS Annual Report). These severe risks are the result of the dialysis treatment itself. Even though dialysis provides a life-saving medical treatment for subjects with end stage renal disease, these treatments severely reduce the quality and quantity of life for subjects. The causal role of dialysis in dialysis-associated morbidity & mortality risks is evidenced by the lower prevalence of these same conditions in subjects with ESRD who receive a kidney transplant rather than remain on dialysis. It has been demonstrated that dialysis alters the physiologic halide balance in subjects, yet it has not been recognized that these imbalanced halide levels can cause cardiovascular disease and/or mortality in dialysis. Data presented herein (FIG. 1) indicate that at least part of the morbidity and mortality rates in dialysis populations are due to these imbalanced halide levels. Embodiments of the invention can reduce the risk of cardiovascular disease and/or mortality in subjects being treated with dialysis by promoting physiologic halide balance in accordance with the specific needs of these subjects.

Dialysis treatments rely on a specialized form of saline solution, termed a dialysate, in order to perform blood filtration in a subject. The composition of this fluid is a key determinant of the subject's bloodstream after the dialysis treatment, and can cause the loss of ions such as Br⁻ and Mg²⁺ from the subject exposed to the dialysate. For example, dialysates contain very little Br⁻, where the ion is only present at background or contaminate levels, such as around 9.5 μM Br⁻ (Muria et al., 2002). These levels are lower than the amount of Br⁻ present in the bloodstream of subjects before starting dialysis. Consequently, at the start of dialysis, the concentration of Br⁻ in a subject's blood is higher than the concentration of Br⁻ in the dialysate. As a result of this concentration gradient, Br⁻ ions are transferred from the subject into the used dialysate, effectively reducing the concentration of Br⁻ in the subject (Muria et al., 2002). This loss effect also occurs with Mg²⁺, as dialysates contain little Mg²⁺ and subjects on dialysis experience low levels of Mg²⁺ in their blood (Sakaguchi et al., 2013).

Mg-deficiency is associated with increased risk of mortality in subjects on dialysis (Sakaguchi et al., 2013). Subjects with serum Mg²⁺ levels between 2.5 and 3.0 mg/dl have shown the lowest risk of developing cardiovascular disease (Sakaguchi et al., 2013). In some subjects with hyperphosphatemia, low Mg²⁺ levels can promote risk of calcification, including vascular calcification (Sakaguchi et al., 2014). Thus, practice of the invention within a subject on dialysis requires the consideration of circulating Mg²⁺ levels as well as circulating Br⁻ levels in said subject.

Since both Br⁻ and Mg²⁺ levels can influence cardiovascular disease and mortality in a subject, without being bound by theory, correcting the level of one ion while ignoring the other ion only provides a limited ability to reduce morbidity and mortality risk in a subject on dialysis. For example, if a dialysate only prevented Mg²⁺ loss in a subject yet did not prevent Br⁻ loss, the subject would still be at risk due to the ongoing Br-deficiency. Alternatively, correcting Br⁻ levels without addressing Mg²⁺ would similarly fail to adequately reduce the subject's risk of developing a co-morbidity or mortality. Indeed, in preclinical studies performed by the inventors, while correcting Br⁻ levels alone significantly improved survival (FIG. 1), it did not completely prevent the occurrence of mortality events. Thus, in order to effectively mitigate a subject's risk of developing a dialysis-associated morbidity or mortality event, the circulating levels of Br⁻ and Mg²⁺ have to be simultaneously maintained at adequate levels. Due to this synergistic activity between Br⁻ and Mg²⁺, embodiments of the invention comprising both Br⁻ and Mg²⁺ can display enhanced effectiveness in preventing cardiovascular disease, kidney disease, or a combination thereof in a subject, such as a subject receiving dialysis.

Some embodiments of the invention comprise therapeutically effective and/or physiologically balanced amounts of Br⁻, Cl⁻, and Mg²⁺. Some embodiments of the invention comprise a dialysate. Some embodiments comprise a dialysate that can be used in acute dialysis or in maintenance dialysis. Some embodiments comprise a dialysate that contains an amount between about 30 μM and about 1 mM Br⁻, an amount between about 80 mM and about 200 mM Cl⁻, and an amount between about 0.75 mM and about 3 mM Mg²⁻. Some embodiments comprise only those amounts of Mg²⁺ required to maintain a subject's serum Mg²⁺ between about 2.5 mg/dl and about 3.0 mg/dl. Some embodiments comprise only those amounts of Br⁻ required to maintain a subject's serum Br⁻ between about 30 μM and about 1 mM. Non-limiting examples of additional minerals and compounds that can also be formulated in the dialysate embodiment comprise bicarbonate, potassium, sodium, calcium, glucose, dextrose, acetate, citric acid, and a combination thereof.

Some embodiments of the invention comprise methods of preventing the development of a morbidity or mortality event in a subject. In some embodiments, the subject is being treated with dialysis. Some embodiments comprise performing dialysis on the subject using a dialysate that contains an amount between about 30 μM and about 1 mM Br⁻, an amount between about 80 mM and about 200 mM Cl⁻, and an amount between about 0.75 mM and about 3 mM Mg²⁺. In some embodiments, the subject can be deficient in Br⁻, Mg²⁺, or a combination thereof. Some embodiments can correct or prevent Mg-deficiency in a subject. Some embodiments can correct or prevent Mg-deficiency as well as Br-deficiency in a subject. In some embodiments, Mg²⁺ levels can be measured in the blood, serum, plasma, urine, or used dialysate of a subject. In some embodiments, Br⁻ levels can be measured in the blood, serum, plasma, urine, or used dialysate of a subject.

Dialysis modalities comprise acute dialysis, hemodialysis, peritoneal dialysis, or home hemodialysis, which are differentiated by how the dialysis is performed and where the dialysis treatment occurs. For example, acute dialysis can be performed on a subject who is experiencing an acute kidney injury. For example, hemodialysis involves performing dialysis on a subject's blood outside the body, also termed extracorporeal blood filtration. As another example, peritoneal dialysis uses one of the subject's biologic membranes (the peritoneal membrane) to perform the filtration. As still another example, home hemodialysis occurs when hemodialysis is performed in the personal home of a subject, rather than a hospital or dialysis clinic. Embodiments of the invention can be administered to subject receiving acute dialysis, hemodialysis, peritoneal dialysis, or home hemodialysis.

Some embodiments of the invention comprise physiologically effective amounts of Br⁻, Cl⁻, and Mg²⁺. Some embodiments of the invention comprise a dialysate comprising an amount between about 30 μM and about 1 mM Br⁻, an amount between about 80 mM and about 200 mM Cl⁻, and an amount between about 0.75 mM and about 3 mM Mg²⁺. In some embodiments, the amount of Br⁻ is 30 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 170 μM, 175 μM, 180 μM, 185 μM, 190 μM, 195 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1 mM, or any other amount between about 30 μM and about 1 mM. In some embodiments, the amount of Cl⁻ is 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, or any other amount between about 80 mM and about 200 mM. In some embodiments, the amount of Mg²⁺ is 0.75 mM, 0.80 mM, 0.85 mM, 0.90 mM, 0.95 mM, 1.05 mM, 1.05 mM, 1.10 mM, 1.15 mM, 1.20 mM, 1.25 mM, 1.30 mM, 1.35 mM, 1.40 mM, 1.45 mM, 1.50 mM, 1.55 mM, 1.60 mM, 1.65 mM, 1.70 mM, 1.75 mM, 1.80 mM, 1.85 mM, 1.90 mM, 1.95 mM, 2.00 mM, 2.05 mM, 2.10 mM, 2.15 mM, 2.20 mM, 2.25 mM, 2.30 mM, 2.35 mM, 2.40 mM, 2.45 mM, 2.50 mM, 2.55 mM, 2.60 mM, 2.65 mM, 2.70 mM, 2.75 mM, 2.80 mM, 2.85 mM, 2.90 mM, 2.95 mM, 3.0 mM, or any other amount between about 0.75 mM and about 3.0 mM. Some embodiments comprise only those amounts of Br⁻ required to maintain a subject's serum Br⁻ between about 30 μM and about 1 mM. Some embodiments comprise only those amounts of Mg²⁺ required to maintain a subject's serum Mg²⁺ between about 2.5 mg/dl and about 5.0 mg/dl.

Some composition embodiments of the invention are formulated as a dialysate and used to perform dialysis on a subject. In some embodiments, the dialysate also comprises an amount of bicarbonate, potassium, sodium, calcium, glucose, dextrose, acetate, citric acid, or any combination thereof. Some embodiments comprise a physiologically balanced amount of Br⁻, Cl⁻, Mg²⁺, and one or more of an amount of bicarbonate, potassium, sodium, calcium, glucose, dextrose, acetate, citric acid, or any combination thereof. Some embodiments of the invention comprise a dialysate or replacement fluid. An example of a dialysate comprises a solution formulated for use during acute dialysis and maintenance dialysis. An example of a replacement fluid comprises a solution formulated for use during acute dialysis. Some embodiments comprise concentrated dialysate fluids that upon dilution yields a final dialysate comprising an amount between about 30 μM and about 1 mM Br⁻, an amount between about 80 mM and about 200 mM Cl⁻, and an amount between about 0.75 mM and about 3 mM Mg²⁺. Some embodiments comprise a dry powder that upon reconstitution with a biocompatible fluid yields a final dialysate comprising an amount between about 30 μM and about 1 mM Br⁻, an amount between about 80 mM and about 200 mM Cl⁻, and an amount between about 0.75 mM and about 3 mM Mg²⁺. Some embodiments comprise a composition that is mixed with another composition to yield a final dialysate comprising an amount between about 30 μM and about 1 mM Br⁻, an amount between about 80 mM and about 200 mM Cl⁻, and an amount between about 0.75 mM and about 3 mM Mg²⁺, such as but not limited to an acidified embodiment of the compositon that is combined with a basic composition to yield a final dialysate comprising an amount between about 30 μM and about 1 mM Br⁻, an amount between about 80 mM and about 200 mM Cl⁻, and an amount between about 0.75 mM and about 3 mM Mg²⁺. As another non-limiting embodiment, an embodiment can comprise a basic (eg high pH) composition embodiment that is combined with an acidic composition to yield a final dialysate comprising an amount between about 30 μM and about 1 mM Br⁻, an amount between about 80 mM and about 200 mM Cl⁻, and an amount between about 0.75 mM and about 3 mM Mg²⁺.

Some embodiments of the invention comprise methods of preventing the development of a morbidity or mortality event in a subject. Some embodiments comprise performing dialysis on the subject using compositions as described herein. For example, embodiments comprise performing dialysis on a subject using a dialysate or replacement fluid that contains an amount between about 30 μM and about 1 mM Br⁻, an amount between about 80 mM and about 200 mM Cl⁻, and an amount between about 0.75 mM and about 3 mM Mg²⁺. In some embodiments, the subject can be deficient in Br⁻, Mg²⁺, or a combination thereof. Some embodiments can correct or prevent Mg-deficiency in a subject. Some embodiments can correct or prevent Mg-deficiency as well as Br-deficiency in a subject. In some embodiments, Mg²⁺ levels can be measured in the blood, serum, plasma, urine, or used dialysate of a subject. In some embodiments, Br⁻ levels can be measured in the blood, serum, plasma, urine, or used dialysate of a subject.

Some embodiments of the invention comprise a method of preventing cardiovascular disease or mortality in a subject by administering a composition as described here. In some embodiments, the subject is being treated with dialysis. Some embodiments of the invention comprise a method of preventing cardiovascular disease or mortality in a subject, the method comprising performing dialysis on the subject against a dialysis fluid comprising a sufficient amount of Br⁻ to maintain the subject's serum Br⁻ between about 30 μM and about 1 mM and a sufficient amount of Mg²⁺ to maintain the subject's serum Mg²⁺ between about 2.5 mg/dl and about 3.0 mg/dl.

### Topical Applications of Invention

Embodiments of the invention can be topically administered to subjects. Non-limiting examples of such applications comprise wound irrigation, cleaning, or moistening; bandages; skin products; surgical wound wash; eye drops; nasal irrigation; and ear drops. Topical use of the invention provides a balanced halide solution, which contrasts with the Br-deficient saline solutions in current use that can alter the physiologic halide balance at the tissue site of application. Administration of a balanced halide solution as described herein can improve overall tissue health at the site of application. When compared to Br-deficient 0.9% saline, embodiments of the invention offer improved ability to promote tissue health in subjects.

Some embodiments of the invention can be topically applied to the eye or a portion of the eye of a subject. In some embodiments, compositions of the invention are administered to a subject with eye disease. In some embodiments, the eye disease comprises a retinal disease, a neurodegenerative eye disease, macular degeneration, glaucoma, dry eye disease, or keratoconus. In some embodiments, and considering the above described injectable embodiments of the invention, compositions of the invention are administered to the eye of a subject using a needle, syringe, tube, dropper, bottle, contact lens, or combination thereof. For example, some embodiments can be applied as eye drops to a subject's eye. As another example, some embodiments can be topically applied to a subject's cornea or an exposed portion of eye tissue during an eye surgery, such being applied to the lens of an eye during cataract surgery. Some embodiments can be used by a subject before or after an eye surgery. For example, compositions of the invention can be administered to a subject who receives laser assisted in situ keratomileusis (LASIK). As another example, compositions of the invention can be administered to a subject who receives cataract surgery.

Some embodiments of the invention can be applied into a subject's ear. Some embodiments can be topically applied into a subject's ear. Some embodiments of the invention can be formulated as ear drops applied through a dropper into a subject's ear. Some embodiments of the invention can be used to treat or prevent an infection in a subject or to dry out a subject's ear. Some embodiments can contain additional drugs or molecules to assist in treating a subject's ear. Non-limiting examples of drugs and molecules that can be co-administered with an embodiment to a subject's ear comprise ciprofloxacin, dexamethasone, gentamicin, hydrocortisone, neomycin, gramicidin, dexamtheasone sodium metasulfobenzoate, phenylethyl alcohol, triamcinolone acetone, nystatin, alcohol, acetic acid, Ciprodex, Gentisone HC, Ciproxin HC, Sofradex, Kenacomb, and Aqua Ear.

Some embodiments of the invention can be applied into a subject's nasal passageways, airways, or lungs. Some embodiments can be sprayed, squirted, or poured into the subject's nostrils. Some embodiments can be inhaled into a subject's airways, lungs, or a combination thereof. Embodiments can be intranasally applied to a subject in order to treat or prevent congestion, infection, or inflammation in a subject. Embodiments suitable for intranasal administration can co-administer a drug to a subject, with non-limiting examples of such drugs comprising decongestents and anti-histamines. Some embodiments can be administered to a subject with cystic fibrosis. For example, some embodiments can be administered via inhalation to a subject with cystic fibrosis in order to restore, maintain or promote a proper halide balance in the subject's airways, lungs, or a combination thereof.

Some embodiments of the invention are applied to intact, broken, cut, incised, wounded, burned, aged, diseased, or damaged skin of a subject. Some embodiments of the invention can be applied to a wound, burn, or ulcer in a subject. Embodiments can be applied to acute wounds, chronic wounds, infected wounds, surgical wounds, venous ulcers, burns, diabetic ulcers, chronic ulcer, pressure ulcer, or mouth sore. Some embodiments of the invention are topically applied to a wound that is caused by a trauma, an aging process, a disease, or a surgical operation. In some embodiments, the wound, burn, or ulcer can be caused by a disease or a disease treatment with non-limiting examples comprising diabetes, cancer chemotherapy, and cancer radiation treatments. Both Br⁻ and Cl⁻ are very important for tissue development, acting in part on the deposition and assembly of collagen IV networks within extracellular basement membranes (Cummings et al., 2016; McCall et al., 2014). Embodiments of the invention can restore, maintain or promote a physiologically balanced halide environment within a wound, burn, or ulcer. Embodiments of the invention can be used to restore, maintain or promote tissue development in a subject. Embodiments can be used to prevent infection in a wound, burn, or ulcer. Embodiments of the invention can be used to wash, irrigate, debride, or moisten a wound, burn, or ulcer. Embodiments of the invention can be combined with a drug, medical device, organ, tissue, or tissue product when treating a wound, burn, or ulcer. Non-limiting examples of drugs, medical devices, organs, tissues, or tissue products that can be co-administered with an embodiment to a wound, burn, or ulcer comprise wound washes, preservatives, osmotic agents, debriding agents, anti-infective agents, antibiotic agents, antifungal agents, engineered tissues, cultured tissues, harvested human tissue, animal tissue, autologous tissue, scaffolds, acellular scaffolds, matrices, hydrating agents, absorbants, hemostatic agents, sutures, hydrophobic dressings, bandages, negative pressure bandages, gauze, plugs, powders, patches, collagen-comprising products, injectable fillers, harvested tissues, engineered tissues, cultured tissues, human tissues, animal tissues, autologous tissues, ligaments, tendons, blood, stem cells, progenitor cells, skin, and combinations thereof.

Some embodiments of the invention can be isotonic. Other embodiments can be hypotonic. Still other embodiments can be hypertonic. Tonicity of the embodiment can be adjusted with an osmotic agent or with sodium chloride, provided that the final Cl⁻ and Br⁻ concentrations in the final product remain within physiologic balance. Non-limiting examples of osmotic agents comprise dextrose, lactose, and sodium bicarbonate. The tonicity can be controlled during manufacturing of the embodiment. Alternatively, an osmotic diluent can be added to the embodiment in a hospital or pharmacy prior to administration to a subject. Embodiments that are injected into an interstitial tissue, such as intramuscular injection, or eye injection can require the final composition to be isotonic prior to injection.

Many embodiments of the invention are suitable for topical administration to a subject. Non-limiting examples of such embodiments comprise solutions, lotions, creams, ointments, gels, pastes, sprays, liquids, washes, hydrating agents or solutions, and perfusing agents or solutions. One skilled in the art will appreciate that all these embodiments are unified by comprising a physiologically balanced amount of Br⁻ and Cl⁻.

Embodiments of the invention comprise a bandage that is moistened with a fluid containing 100 μM NaBr and 0.9% NaCl saline. For example, such a bandage can comprise gauze pads, NaCl, NaBr and water or an aqueous fluid. For example, such a bandage can comprise:
3×3 inch gauze pads
8.99976 g of NaCl (at least USP grade)
10.3 mg of NaBr (at least USP grade)
2 L of water

To create a suitable salt reagent, the NaBr and NaCl are first added to 1 L of water, filter purified from solution, and dried to a powder. Subsequently, 900 mg of the reagent is dissolved in 70 ml of water before the final volume is adjusted to 100 ml, yielding a solution with 100 μM NaBr and 0.9% NaCl. This solution can be filter sterilized. To assemble the final embodiment, 3-5 ml of the sterilized solution is added to the gauze. The moistened gauze can then be sealed in a water-proof packaging and terminally sterilized before use. This embodiment can be applied to an open wound as a moist bandage.

### Use of Invention on a Medical Device

Medical devices are frequently contacted with Br-deficient saline solutions, with non-limiting examples comprising washing contact lenses in saline solutions, moistening wound bandages with saline, and flushing medical tubes with saline. Contact between these devices and Br-deficient 0.9% saline exposes the device to halide concentrations that are not physiologically relevant. This can be detrimental to subjects. For example, contact lenses soaked in Br-deficient saline can administer higher concentrations of Cl⁻ to a subject if placed on the cornea without washing off the saline. Alternatively, flushing a medical tube with Br-deficient 0.9% saline, such as flushing a central line after administering a chemotherapeutic agent to a subject with cancer or flushing a catheter, can alter the physiologic halide balance in the subject. As another example, Br-deficient 0.9% saline can be used to moisten a bandage for application to a large open wound in a battlefield or first-responder situation. Such wounds require a moist environment, yet the Br-deficient 0.9% saline represents a non-physiological amount of Cl⁻ that inherently alters the wound environment in a way that can impede healing. In all these examples, the use of Br-deficient 0.9% saline can impair the ability of the medical device to be safely and effectively used in the subject. In contrast, use of the invention can allow the medical device to better mimic physiologic halide balance, which can improve biocompatibility of the device and improve outcomes in the subject.

Embodiments of the invention can be useful for washing, hydrating, perfusing, or cleaning a medical device. Embodiments of the invention can be used to wash, hydrate, perfuse, or clean a device used in dialysis. This washing, hydrating, perfusing, or cleaning action can be performed before and/or after the device is used with a subject. Non-limiting examples of such devices comprise access ports, catheters, intravenous and intra-arterial lines, dialysis machines, dialysis tubing, dialysis bags, bandages, water filtration systems, chairs, and carts.

In some method embodiments, the composition is contacted with a medical device. In some method embodiments, the medical device comprises a surgical device, a syringe, a needle, a suture, a contact lens, a medical device used in dialysis, a dialysis machine, a port, a fistula, a catheter, a graft, a machine used in the medical care of a subject, a component of a machine used in the medical care of a subject, a stent, a plug, a patch, an injection filler, a powder, a bandage, a gauze, a negative pressure device, a hydrophobic dressing, a hemostatic agent, an absorbent, a hydrating agent, an osmotic agent, a preservative, a wash, a lotion, a cream, a gel, a paste, an ointment, a rinse, an eye drop, an ear drop, a spray, a nasal spray, a matrix, a scaffold, an acellular scaffold, a collagen-based device, a camera, a pill camera, a tube, a connector, a needlefree connector, a closed system, transfer device, a hemodynamic monitoring system, a bag, a balloon, an intravenous line, a central line, an arterial line, or a combination thereof. In some embodiments, the composition is used to cleanse, wash, moisten, hydrate, perfuse, or lubricate the medical device.

In some embodiments, compositions of the invention are contacted with an organ, a tissue, or a tissue product. Non-limiting examples of organs, tissues, or tissue products comprise harvested tissues, engineered tissues, cultured tissues, human tissues, animal tissues, autologous tissues, bone, ligaments, tendons, vascularized organs, lung, kidney, heart, heart tissue, liver, pancreas, corneas, blood, stem cells, progenitor cells, skin, dura mater, oocytes, semen, and combinations thereof. In some embodiments, the composition is used to cleanse, wash, moisten, hydrate, perfuse, or lubricate the organ, tissue, or tissue product.

### Location of Use

Embodiments of the invention can be used in a hospital or critical care setting. Non-limiting examples of such embodiments comprise bagged intravenous (IV) solutions, irrigation solutions, washes, hydrating solutions, and perfusing solutions. Non-limiting examples of uses for the invention in a hospital comprise injection applications; topical applications; washing or perfusion of catheters, central lines, or other tubes; replacement solutions in continuous renal replacement therapy; and washing, hydrating, or perfusing components of dialysis delivery systems. In these cases, embodiments can administer a physiologically balanced halide solution to a subject, in order to accelerate the recovery of the subject. For a hospital or critical care facility, this improvement of patient care can result in improved performance metrics such as fewer hospitalization days per patient, higher patient turnover, and reduced readmissions.

Embodiments of the invention can be used in a medical clinic. Non-limiting examples comprise vaccine or drug injections; washing, hydrating, perfusing cleansing, irrigation, or other topical applications; and applications within the field of maintenance dialysis, such as washing components of a dialysis delivery device, access port, or catheter. Use of the inventions described herein, for example in a clinic setting, can result in improved outcomes in subjects, reducing the chances that the subject will require subsequent admission to a hospital.

Embodiments of the invention can be used at home. Non-limiting examples of use comprise first aid applications, such as but not limited to moistening a bandage, irrigating a wound, or hydrating a large open wound; and contact lens cleaning. Some embodiments for home use can be manufactured into packets, vials, bottles, or aerosol cans. The invention can be useful for promoting halide balance in a subject at home, yielding more effective self-treatment and reducing the chance that the subject will require additional care at a medical facility.

Embodiments of the invention can be used in a field setting, such as a battlefield, a military hospital, a medical transport, or the scene of an accident. Non-limiting examples of use comprise first aid applications, such as but not limited to moistening a bandage, irrigating a wound, or hydrating a large open wound; injection applications such as but not limited to maintaining adequate blood pressure in a subject, administering a blood substitute or artificial blood product, or administering a drug; or washing, cleansing, hydrating, perfusing, or moistening medical devices. The invention is appropriate for civilian as well as military use. The invention can be packaged in any means that is suitable or preferred for field use, where non-limiting examples comprise packets, vials, bottles, aerosol cans, bags, or pre-filled syringes. In a field-use setting, rapid treatment of emergency conditions is required in order to accelerate the subject's recovery time. The invention can restore halide balance in a subject in order to more quickly restore physiologic halide levels in the subject, allowing the subject to focus energies on healing the primary injury.

### Veterinary Applications

Br-deficient saline solutions are routinely used in the field of veterinary medicine. Embodiments as described herein are useful for veterinary applications, since maintaining a physiologic balance of Br⁻ and Cl⁻ is important for all animals. For example, a physiologic balanced amount of Br⁻ and Cl⁻ in a composition, such as embodiments of the invention comprises an amount of Br⁻ that is between about 30 μM and about 1 1M and an amount of that is between about 17.1 mM and about 200 mM. Thus, the principle of maintaining proper halide balance is important in veterinary medicine.

Compositions of the invention described herein can be administered to cows to prevent or treat heart failure, also called brisket disease. Particularly at high altitudes, cattle are at risk of developing brisket disease, which can be fatal to the affected cow. In the USA, brisket disease can cause substantial economic losses. The pathogenesis is believed to hinge on the primary development of pulmonary hypertension, also called bovine pulmonary hypertension, leading to the subsequent development of heart failure. In some cases, brisket disease may develop in a cow that has been treated for respiratory disease. Compositions of the invention described herein can decrease blood pressure in a subject, such as but not limited to a cow, to improve or prevent symptoms of heart failure or brisket disease. Compositions of the invention described herein can be administered to a cow or herd to improve the health of the cow of herd. Compositions of the invention described herein can be administered to a cow or herd through food or drink. Some embodiments can be administered to a cow or herd as a drug, a vaccine, an inoculation, a shot, an injectable drug, a pill, a powder, or a patch. Some compositions of the invention can be combined with another veterinary drug or vaccine for administration to a cow or herd. Some compositions of the invention described herein can be administered only once to a cow or herd. Some compositions of the invention can be periodically administered to a cow or herd, such as but not limited to every other day, once a week, or once a month. Some compositions of the invention described herein can be administered daily or even more than once per day to a cow or herd. Some compositions can be administered to breeder cattle in order to prevent heart disease development in the offspring of the breeders. Compositions of the invention described herein can be administered in a field, roofed enclosure, pen, or feedlot. Some compositions can be administered to a cow or herd by a veterinarian. Other compositions described herein can be administered to a cow or herd by a cattleman, farmer, or rancher. Compositions of the invention described herein can be manufactured, packaged, and distributed as a pre-mixed food or food supplement for cattle.

Poultry production is exposed to flock damage from ascites, or fluid accumulation in the peritoneal cavities which can be fatal for the affected bird. Broiler chicken production is particularly exposed to ascite-associated losses. A key cause of ascites in broiler chickens is pulmonary hypertension, while other causes include infection and liver damage. Compositions of the invention described herein can be used to reduce blood pressure in a bird or poultry flock in order to prevent the development of ascites. Compositions of the invention described herein can be administered to a bird or poultry flock to improve the health of the bird or poultry flock. Some compositions described herein can be administered to a poultry flock through food or drink. Some compositions can be administered to a poultry flock as a drug, a vaccine, an inoculation, a shot, an injectable drug, a pill, a powder, or a patch. Some Compositions of the invention described herein can be administered only once to a bird or flock. Some compositions can be periodically administered to a bird or flock, such as but not limited to every other day, once a week, or once a month. Compositions of the invention described herein can be administered daily or even more than once per day to a bird or flock. Compositions of the invention described herein can be administered to breeder chickens in order to prevent ascite development in the offspring of the breeders. Some compositions can be administered in a field, roofed enclosure, pen, or feedlot. Some compositions can be administered to a cow or herd by a veterinarian. Other compositions described herein can be administered to a cow or herd by a farmer or rancher. Compositions of the invention described herein can be manufactured, packaged, and distributed as a pre-mixed food or food supplement for poultry.

Pig production entails protecting pigs from Mulberry heart disease, which displays as weakness and sudden death of the animal. Commonly understood to be caused by selenium deficiency, Mulberry heart disease can occur in animals with sufficient amounts of selenium. This indicates that the full disease etiology is incompletely understood, and suggests that additional dietary and therapeutic interventions are needed to prevent Mulberry heart disease. Embodiments of the invention can improve survival of animals at risk of dying from heart disease (FIG. 1). Compositions of the invention described herein can be administered to pig or herd to prevent Mulberry heart disease. Compositions of the invention described herein can be administered to improve the health of a pig or herd. Compositions of the invention described herein can be administered to a pig or herd through food or drink. Some compositions can be administered to a pig or herd as a drug, a vaccine, an inoculation, a shot, an injectable drug, a pill, a powder, or a patch. Some compositions can be combined with another veterinary drug or vaccine for administration to a pig or herd. Some compositions can be administered only once to a pig or herd. Compositions of the invention described herein can be periodically administered to a pig or herd, such as but not limited to every other day, once a week, or once a month. Some compositions can be administered daily or even more than once per day to a pig or herd. Some compositions can be administered to breeder pigs in order to prevent Mulberry heart disease in the offspring of the breeders. Compositions of the invention described herein can be administered in a field, roofed enclosure, pen, or feedlot. Some compositions can be administered to a pig or herd by a veterinarian. Other compositions can be administered to a pig or herd by a farmer or rancher. Compositions of the invention described herein can be manufactured, packaged, and distributed as a pre-mixed food or food supplement for pigs.

Turkeys can experience sudden death syndrome, perirenal hemorrhage syndrome, hypertensive angiopathy. These conditions can cause mortality within a turkey flock, leading to significant economic damage during the rising and production of turkeys. All of these conditions derive from an underlying cardiovascular disease in the turkeys. Compositions of the invention described herein can be administered to turkeys to prevent and treat cardiovascular disease during the raising and production of turkeys. Compositions of the invention described herein can be administered to improve the health of a turkey or flock. Compositions of the invention described herein can be administered to a turkey or flock through food or drink. Some compositions can be administered to a turkey or flock as a drug, a vaccine, an inoculation, a shot, an injectable drug, a pill, a powder, or a patch. Some compositions can be combined with another veterinary drug or vaccine for administration to a turkey or flock. Some compositions can be administered only once to a turkey or flock. Compositions of the invention described herein can be periodically administered to a turkey or flock, such as but not limited to every other day, once a week, or once a month. Some compositions can be administered daily or even more than once per day to a turkey or flock. Compositions of the invention described herein can be administered to breeder turkeys in order to prevent cardiovascular disease in the offspring of the breeders. Some compositions can be administered in a field, roofed enclosure, pen, or feedlot. Compositions of the invention described herein can be administered to a turkey or flock by a veterinarian. Other embodiments can be administered to a turkey or flock by a farmer or rancher. Embodiments can be manufactured, packaged, and distributed as a pre-mixed food or food supplement for turkeys.

Some embodiments of the invention comprise a composition that can be administered to an animal subject between about 0.0001mg to aboout 0.1 mg Br⁻ per kg body weight of the subject. Non-limiting examples of animal subjects include dog, cat, mouse, rabbit, rat, hamster, guinea pig, mouse, rabbit, rat, hamster, guinea pig, horse, cow, goat, sheep, pig, chicken, or turkey. Compositions of the invention described herein can be administered at about 0.0001 mg, 0.0005 mg, 0.0010 mg, 0.0015 mg, 0.0020 mg, 0.0025 mg, 0.0030 mg, 0.0035 mg, 0.0040 mg, 0.0045 mg, 0.0050 mg, 0.0055 mg, 0.0060 mg, 0.0065 mg, 0.0070 mg, 0.0075 mg, 0.0080 mg, 0.0085 mg, 0.0090 mg, 0.0095 mg, 0.0100 mg, 0.0105 mg, 0.0110 mg, 0.0115 mg, 0.0120 mg, 0.0125 mg, 0.0130 mg, 0.0135 mg, 0.0140 mg, 0.0145 mg, 0.0150 mg, 0.0155 mg, 0.0160 mg, 0.0165 mg, 0.0170 mg, 0.0175 mg, 0.0180 mg, 0.0185 mg, 0.0190 mg, 0.0195 mg, 0.0200 mg, 0.0205 mg, 0.0210 mg, 0.0215 mg, 0.0220 mg, 0.0225 mg, 0.0230 mg, 0.0235 mg, 0.0240 mg, 0.0245 mg, 0.0250 mg, 0.0255 mg, 0.0260 mg, 0.0265 mg, 0.0270 mg, 0.0275 mg, 0.0280 mg, 0.0285 mg, 0.0290 mg, 0.0295 mg, 0.0300 mg, 0.0305 mg, 0.0310 mg, 0.0315 mg, 0.0320 mg, 0.0325 mg, 0.0330 mg, 0.0335 mg, 0.0340 mg, 0.0345 mg, 0.0350 mg, 0.0355 mg, 0.0360 mg, 0.0365 mg, 0.0370 mg, 0.0375 mg, 0.0380 mg, 0.0385 mg, 0.0390 mg, 0.0395 mg, 0.0400 mg, 0.0405 mg, 0.0410 mg, 0.0415 mg, 0.0420 mg, 0.425 mg, 0.0430 mg, 0.0435 mg, 0.0440 mg, 0.0445 mg, 0.0450 mg, 0.0455 mg, 0.0460 mg, 0.0465 mg, 0.0470 mg, 0.0475 mg, 0.0480 mg, 0.0485 mg, 0.0490 mg, 0.0495 mg, 0.0500 mg, 0.0505 mg, 0.0510 mg, 0.0515 mg, 0.0520 mg, 0.0525 mg, 0.0530 mg, 0.0535 mg, 0.0540 mg, 0.0545 mg, 0.0550 mg, 0.0555 mg, 0.0560 mg, 0.0565 mg, 0.0570 mg, 0.0575 mg, 0.0580 mg, 0.0585 mg, 0.0590 mg, 0.0595 mg, 0.0600 mg, 0.0605 mg, 0.0610 mg, 0.0615 mg, 0.0620 mg, 0.0625 mg, 0.0630 mg, 0.0635 mg, 0.0640 mg, 0.0645 mg, 0.0650 mg, 0.0655 mg, 0.0660 mg, 0.0665 mg, 0.0670 mg, 0.0675 mg, 0.0680 mg, 0.0685 mg, 0.0690 mg, 0.0695 mg, 0.0700 mg, 0.0705 mg, 0.0710 mg, 0.0715 mg, 0.0720 mg, 0.0725 mg, 0.0730 mg, 0.0735 mg, 0.0740 mg, 0.0745 mg, 0.0750 mg, 0.0755 mg, 0.0760 mg, 0.0765 mg, 0.0770 mg, 0.0775 mg, 0.0780 mg, 0.0785 mg, 0.0790 mg, 0.0795 mg, 0.0800 mg, 0.0805 mg, 0.0810 mg, 0.0815 mg, 0.0820 mg, 0.0825 mg, 0.0830 mg, 0.0835 mg, 0.0840 mg, 0.0845 mg, 0.0850 mg, 0.0855 mg, 0.0860 mg, 0.0865 mg, 0.0870 mg, 0.0875 mg, 0.0880 mg, 0.0885 mg, 0.0890 mg, 0.0895 mg, 0.0900 mg, 0.0905 mg, 0.0910 mg, 0.0915 mg, 0.0920 mg, 0.0925 mg, 0.0930 mg, 0.0935 mg, 0.0940 mg, 0.0945 mg, 0.0950 mg, 0.0955 mg, 0.0960 mg, 0.0965 mg, 0.0970 mg, 0.0975 mg, 0.0980 mg, 0.0985 mg, 0.0990 mg, 0.095 mg, 0.1000 mg, or any other amount between about 0.0001 mg and about 0.1000 mg Br⁻ per kg body weight of the subject. For example, spontaneously hypertensive rats embodiments of the invention were administered embodiments of the invention comprising a total 6.84 mg Br⁻ per kg of diet. This embodiment was formulated by supplementing AIN76a with a sufficient amount of NaBr to yield a final amount of 6.84 mg Br— per kg of diet. On study day 43, the average rat weight was 340 g. Assuming each rat consumed approximately 25 g of diet per day, then the embodiment administered to each rat approximately 0.0005 mg Br⁻ per kg body weight of the rat. As another example, salt sensitive Dahl rats were administered a diet containing 8% NaCl and a final amount of 243 mg Br⁻ per kg diet. On study day 43, the average rat weight was 396 g. Assuming each rat consumed approximately 25 g of diet per day, then the embodiment administered to each rat approximately 0.0153 mg Br⁻ per kg body weight of the rat.

Some embodiments of the invention can be administered to an animal as a treatment or preventative treatment for kidney disease, such as in a dog or cat. Some embodiments can be administered to improve the survival of a dog, cat, mouse, rabbit, rat, hamster, guinea pig, turkey, chicken, cow, pig, goat, sheep, or horse. Some embodiments can be administered to a flock, herd, or population of animals to improve production yields within the flock, herd, or population.

All embodiments of the invention, such as those described herein, are therefore applicable to veterinary uses. Embodiments as described herein are useful for administration to an animal as a means of promoting, restoring, or maintaining an optimal halide balance in the animal subject. Any human disease or condition described herein for which there is a veterinary counterpart can be treated by an embodiment of the invention. Any human drug, drug ingredient, or medical device that can also be used in veterinary medicine can be co-administered to an animal with an embodiment of the invention. Any human drug, drug ingredient, or medical device that is manufactured for use in veterinary medicine can use an embodiment of the invention during an appropriate manufacturing step. A non-limiting example of an appropriate manufacturing step comprises any manufacturing step where a saline solution is used. Another non-limiting example of an appropriate manufacturing step comprises any manufacturing step where sodium chloride, potassium chloride, or magnesium chloride is used.

### Administering the Invention to Patients on Dialysis

Dialysis treatments remove dietary Br⁻ from patients. For example, one report demonstrates a lowering of serum Br⁻ levels from 20.8 μM before dialysis to 12.0 μM, afterwards (Miura et al., 2002). Without being bound by theory, the pre-dialysis levels are still lower than the average of around 60 μM Br⁻ seen in healthy non-dialysis adults, likely as a result of Br⁻ loss in the previous dialysis treatment. This demonstrated that dietary sources of Br⁻ are apparently insufficient to reverse low Br⁻ levels in between dialysis treatments, underscoring the need for the present invention to restore healthy Br⁻ levels in these patients.

Embodiments comprise determining how much Br⁻ needs to be administered to an individual patient in order to raise the circulating Br⁻ levels from X M to Y M following treatment, as a means of maintaining a serum Br⁻ levels in a healthy range, and administering a determined about of Br⁻ to the subject.

$$( Y molar concentration - X molar concentration ) * ( patient extracellular volume ) * ( 79.9 \frac{g}{mol} Br ) = mass Br to administer$$

For example, one can determine a nutritionally effective amount of Br⁻ needed by a subject who is undergoing dialysis treatments, in order to assist the subject in maintaining a serum Br⁻ concentration of around 60 μM following treatment. Estimating that an individual dialysis patient has approximately 12 μM serum Br⁻ and 6 L of blood, which would equate to a total of 72 μmol of Br⁻ in the subject's serum. If the subject's serum contained 60 μM Br⁻, at total of 360 μmol Br⁻ would be in the subject's serum. This subject would need a nutritional amount of 288 μmol or 23 mg of Br⁻ in order to display a 60 μM serum Br⁻ concentration. Thus, an embodiment of the invention can administer 23 mg Br⁻ as a means of providing a nutritionally effective amount of Br⁻ to the subject.

The above example assumes that the administered embodiment provides the sole source of Br⁻ to the subject in between dialysis treatments. However, it can also be validly assumed that the subject would likely consume additional amounts of Br⁻ from his/her standard diet before the next dialysis treatment. As a result, it should further be assumed that if 23 mg was indeed administered, said subject would likely display higher than 60 μM Br⁻ by the next dialysis treatment. The analysis below accounts for the reasonable dietary Br⁻ intake when calculating a non-limiting example nutritionally effective amount of Br⁻ to administer to a subject.

Patients receiving maintenance dialysis typically consume similar amounts of dietary Br⁻ as the general healthy population. Without wishing to be bound by theory, as a result, their serum Br⁻ levels can oscillate between a rise in serum Br⁻ levels during the 2- or 3-day period between dialysis sessions, due to dietary intake, and a rapid loss of Br⁻ during the dialysis treatment session itself. Without wishing to be bound by theory, a subject in the United States, may consume between 4-16 mg for a 2-day period between dialysis treatments, or between 6-24 mg for a 3-day period. As a consequence, if a patient diligently consumes a Br-rich diet, said patient should successfully replace all the lost Br⁻ by the end of said 3-day period. However, a Br-rich diet is not likely to provide sufficient amounts of Br⁻ over a 2-day period. Moreover, a diet with little available Br⁻, such as a diet high in salt or a diet without an abundance of seafood, nuts, or green leafy vegetables, is unlikely to provide sufficient amounts of Br³¹ . Accounting for the reasonable dietary intake of Br⁻, some embodiments of the invention contain 5 mg of Br³¹ . Administration of this amount of Br⁻ to a subject on dialysis, who possesses approximately 12 μM serum Br⁻ and 6 L of blood following dialysis, should yield a serum Br⁻ concentration of about 22 μM after digesting and absorbing all of the embodied amount of Br³¹ . The effect of practicing the invention in this way can help the subject to supplement the amount of Br⁻ consumed through a normal diet and minimize the period in which the patient is Br-deficient immediately following dialysis.

Thus, embodiments of the invention can comprise a dietary supplement comprising 5 mg of Br³¹ . In embodiments, the Br⁻ can be in the form of NaBr. In embodiments, the composition is administered to or consumed by dialysis patients. This supplement is intended for administration immediately following the dialysis treatment. In embodiments, the injection can be administered before the dialysis treatment or during the dialysis treatment. In the case of hemodialysis, which is performed in a dialysis clinic, the supplement can be administered before the patient leaves the clinic.

### Application of the Invention as a Vitamin for Dialysis Patients

Patients on dialysis are advised to take multivitamins in order to compensate for treatment-induced nutritional imbalances. There are several vitamins that are formulated specifically for dialysis patients, yet in spite of their widespread use, dialysis still places patients at significant risk of developing cardiovascular disease. In other words, the currently available dietary supplements are insufficient for adequately promoting heart health and/or vascular health in patients on dialysis. Consequently, these patients experience high risk of developing cardiovascular disease, in part due to these inadequate vitamins. It has not been known that Br⁻ is important for heart health and/or vascular health, yet the inventors here show that loss of Br⁻ is associated with increased mortality in a rat model of hypertension and kidney disease (FIG. 1). Dialysis treatments simultaneously remove Br⁻ from patients while elevating patient risk of developing cardiovascular disease. Embodiments of the invention comprise a composition with enhanced ability to promote heart health and kidney health in a patient on dialysis. That is, while other dialysis vitamins that lack Br⁻ have been incapable of preventing cardiovascular disease in dialysis patients, the composition provides a more complete set of nutrients that are important for dialysis patients and thereby better promotes heart and kidney health in this population. Embodiments of the invention comprise compositions comprising bromine and at least one of magnesium, Vitamin B₁, vitamin B₂, vitamin B₃, vitamin B₅, vitamin B₆, vitamin B₇, vitamin B₉, vitamin B₁₂, vitamin C. For example, compositions described herein can comprise at least two or more of the following:
About 5 mg Br⁻ (NaBr),
About 100 mg magnesium (MgCl₂),
About 1.2 mg of vitamin B₁ (thiamine),
About 1.7 mg of vitamin B₂ (riboflavin),
About 20 mg of vitamin B₃ (niacin),
About 10 mg of vitamin B₅ (pantothenic acid),
About 50 mg of vitamin B₆ (pyridoxine),
About 300 μg of vitamin B₇ (biotin),
About 0.8 mg of vitamin B₉ (folic acid),
About 6 μg of vitamin B₁₂ (cobalamin), and
About 100 mg of vitamin C (ascorbic acid).

Moreover, some nutritional embodiments of the invention also comprise magnesium, vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid), vitamin B₁₂ (cobalamin), vitamin C (ascorbic acid), vitamin E (tocopherol), selenium, zinc, iron, vitamin D, or a combination thereof. In embodiments, the nutritional composition comprises one or more of the following:
About 50 mg-600 mg magnesium,
About 1.2 mg-1.5 mg of vitamin B₁ (thiamine),
About 1.3 mg-1.7 mg of vitamin B₂ (riboflavin),
About 16 mg-20 mg of vitamin B₃ (niacin),
About 10 mg of vitamin B₅ (pantothenic acid),
About 1.7 mg-100 mg of vitamin B₆ (pyridoxine),
About 30 mg-300 μg of vitamin B₇ (biotin),
About 0.4 mg-5 mg of vitamin B₉ (folic acid),
About 2.4 μg-2 mg of vitamin B₁₂ (cobalamin),
About 60 mg-100 mg of vitamin C (ascorbic acid),
About 30 I.U. of vitamin E (tocopherol),
About 70 μg of selenium,
About 11 mg-25 mg of zinc,
About 18 mg-29 mg of iron, and/or
About 400 I.U.-2000 I.U. of vitamin D.
   Use of Embodiments in Subjects with Cancer

Cancer treatments can deplete Br⁻ from a subject. For example, Weber and colleagues (1984) documented the depletion of Br⁻ from four patients with acute myelogenous leukemia after induction of chemotherapy treatments. Moreover, it is known that exposure to chemotherapeutics or radiation, during the course of cancer treatments, can lead to elevated risk of developing a form of cardiovascular disease or kidney disease. Non-limiting examples of the forms of these diseases that can be triggered by cancer treatments comprise Q-T prolongation, hypertension, heart failure, cardiomyopathy, thromboembolism, edema, ischemia, atrial fibrillation, cardiac shock, bradycardia, and kidney injury. Non-limiting examples of specific chemotherapeutics that can cause these forms of disease comprise arsenic trioxide, bevacizumab, bortezomib, cisplatin, anthracyclines, doxorubicin, fluorouracil 5-FU, imatinib, interleukin alpha-2b, lapatinib, lenalidomide, melphalan, mitomycin, mitoantrone, pazopanib, sorafenib, sunitinib, thalidomide, trastuzumab, carmustine, and methotrexate.

Embodiments of the invention can administer nutritionally effective amounts of Br⁻ to a subject receiving cancer treatment. Such practice of the invention can promote heart health, kidney health, or a combination thereof in a subject with cancer or who has received a cancer treatment. The invention can assist in preventing the development of a form of cardiovascular disease, kidney disease, or a combination thereof in a subject with cancer. Alternatively, the invention can assist in the recovery of the subject who has been treated with one or more cancer treatment regimens. The subject can be receiving radiation treatments, surgery, chemotherapy, or a combination thereof. In some embodiments, the invention can be administered to a subject before, after, or during an individual cancer treatment. The cancer treatment can be chemotherapy, surgery, radiation, or a combination thereof. In some embodiments, the invention can be administered to a subject before the subject is placed onto a cancer treatment regimen. For example, after a subject is diagnosed with cancer yet before the subject begins a set of cancer treatments or surgery, the subject can be administered one or more embodiments of the invention in preparation for the upcoming cancer treatments or surgery. In some embodiments, the invention can be administered to a subject after the subject has completed a cancer treatment regimen. For example, a subject who has completed a specified chemotherapy, radiation, or surgical treatment regimen may still be administered embodiments of the invention in order to assist the recovery of the subject. In another example, a subject can be administered embodiments of the invention days, weeks, months, or years after completing a cancer treatment regimen in order to prevent the development of cardiovascular disease, kidney disease, or a combination thereof. Embodiments of the invention can be similarly administered to subjects who do not complete a cancer treatment regimen, or who change from one cancer treatment regimen to another.

In some embodiments, the invention can comprise an effective amount of Br⁻ and a chemotherapeutic agent or radiation therapy, for co-administration a subject. Non-limiting examples of chemotherapy and radiotherapy that may be used with embodiments of the invention comprise methotrexate, paclitaxel, brentuximab, brentuximab vedotin, anthracyclines, doxorubicin, doxorubicin lipid complex, fluorouracil, fluorouracil 5-FU, everolimus, pemetrexed, melphalan, pamidronate, anastrozole, exemestane, nelarabine, ofatumumab, bevacizumab, belinostat, tositumomab, carmustine, bleomycin, blinatumomab, bosutinib, busulfan, alemtuzumab, irinotecan, vandetanib, bicalutamide, lomustine, daunorubicin, daunorubicin lipid complex, clofarabine, cabozantinib, dactinomycin, cobimetinib, ramucirumab, cytarabine, cytarabine lipid complex, cytoxan, cyclophosphamide, decitabine, dexamethasone, docetaxel, hydroxyurea, decarbazine, leuprolide, epirubicin, oxaliplatin, asparaginase, asparaginase Erwinia chrysanthemi, estramustine, cetuximab, vismodegib, amifostine, etoposide, flutamide, toremifene, panobinostat, fulvestrant, letrozole, degarelix, fludarabine, pralatrexate, floxuridine, obinutuzumab, gemcitabine, afatinib, imatinib, imatinib mesylate, carmustine, eribulin, trastuzumab, altretamine, topotecan, palbociclib, ponatinib, idarubicin, ifosfamide, ibrutinib, axitinib, interferon alpha-2a, peginterferon alpha-2a, gefitinib, romidepsin, ixabepilone, ruxolitinib, cabazitaxel, ado-trastuzumab emtansine, pembrolizumab, caifilzomib, lenvatinib, chlorambucil, sargramostim, cladribine, trifluridine, tipiracil, leuprolide, olaparid, mitotane, vincristine, vincristine lipid complex, procarbazine, megestrol, trametinib, mesna, strontium-89 chloride, mechlorethamine, mitomycin, mitoantrone, busulfan, gemtuzumab ozogamicin, vinorelbine, filgrastim, pegfilgrastim, sorafenib, nilutamide, pentostatin, tamoxifen, mitoxantrone, sonidegib, pegaspargase, denileukin diftitox, nivolumab, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine, dabrafenib, erlotinib, bexarotene, decarbazine, docetaxel, temozolomide, thiotepa, thalidomide, bacillus calmette-guerin (BCG) vaccine, temsirolimus, bendamustine hydrochloride, triptorelin, arsenic trioxide, lapatinib, dinutuximab, valrubicin, panitumumab, vinblastine, bortezomib, tretinoin, azacitidine, pazopanib, teniposide, leucovorin, crizotinib, capecitabine, enzalutamide, ipilimumab, trabectedin, ziv-afibercept, streptozocin, vemurafenib, ibritumomab tiuxetan, goserelin, vorinostat, everolimus, idelalisib, ceritinib, abiraterone, liposomes, deoxyribonucleic acid agents, ribonucleic acid agents, x-rays, gamma rays, charged particles, and combinations thereof.

### Application of the Invention as a Dietary Salt

Elevated salt intake is associated with hypertension, which can lead to cardiovascular disease and/or kidney disease. Consequently, patients with hypertension are frequently advised to limit their salt intake. However, salt is ubiquitously used in food preparation, seasoning, and preservation. Moreover, many individuals prefer the taste of salted foods compared to unsalted foods. In summary, adhering to a low-salt diet can be challenging for some individuals.

In some embodiments, the invention comprises a table salt comprising Br⁻ and Cl⁻ ions, the amount of Br⁻ are an amount between about 0.0338% and about 4.613% of the amount of Cl⁻ on a molar basis. For example, some embodiments comprise an amount of Br⁻ that is about 0.0338%, 0.04%, 0.05%, 0.06%, 0.07%, 0.08%, 0.09%, 0.10%, 0.12%, 0.14%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, 1.00%, 1.02%, 1.04%, 1.06%, 1.08%, 1.10%, 1.12%, 1.14%, 1.16%, 1.18%, 1.20%, 1.22%, 1.24%, 1.26%, 1.28%, 1.30%, 1.32%, 1.34%, 1.36%, 1.38%, 1.40%, 1.42%, 1.44%, 1.46%, 1.48%, 1.50%, 1.52%, 1.54%, 1.56%, 1.58%, 1.60%, 1.62%, 1.64%, 1.66%, 1.68%, 1.70%, 1.72%, 1.74%, 1.76%, 1.78%, 1.80%, 1.82%, 1.84%, 1.86%, 1.88%, 1.90%, 1.92%, 1.94%, 1.96%, 1.98%, 2.00%, 2.02%, 2.04%, 2.06%, 2.08%, 2.10%, 2.12%, 2.14%, 2.16%, 2.18%, 2.20%, 2.22%, 2.24%, 2.26%, 2.28%, 2.30%, 2.32%, 2.34%, 2.36%, 2.38%, 2.40%, 2.42%, 2.44%, 2.46%, 2.48%, 2.50%, 2.52%, 2.54%, 2.56%, 2.58%, 2.60%, 2.62%, 2.64%, 2.66%, 2.68%, 2.70%, 2.72%, 2.74%, 2.76%, 2.78%, 2.80%, 2.82%, 2.84%, 2.86%, 2.88%, 2.90%, 2.92%, 2.94%, 2.96%, 2.98%, 3.00%, 3.02%, 3.04%, 3.06%, 3.08%, 3.10%, 3.12%, 3.14%, 3.16%, 3.18%, 3.20%, 3.22%, 3.24%, 3.26%, 3.28%, 3.30%, 3.32%, 3.34%, 3.36%, 3.38%, 3.40%, 3.42%, 3.44%, 3.46%, 3.48%, 3.50%, 3.52%, 3.54%, 3.56%, 3.58%, 3.60%, 3.62%, 3.64%, 3.66%, 3.68%, 3.70%, 3.72%, 3.74%, 3.76%, 3.78%, 3.80%, 3.82%, 3.84%, 3.86%, 3.88%, 3.90%, 3.92%, 3.94%, 3.96%, 3.98%, 4.00%, 4.02%, 4.04%, 4.06%, 4.08%, 4.10%, 4.12%, 4.14%, 4.16%, 4.18%, 4.20%, 4.22%, 4.24%, 4.26%, 4.28%, 4.30%, 4.32%, 4.34%, 4.36%, 4.38%, 4.40%, 4.42%, 4.44%, 4.46%, 4.48%, 4.50%, 4.52%, 4.54%, 4.56%, 4.58%, 4.60%, about 4.613%, or any other amount that is between about 0.0338% and about 4.613% of the amount of Cl⁻ on a molar basis. In some embodiments, the table salt is iodized. In other embodiments, the table salt is non-iodized, kosher, or pickling salt. In some embodiments, the table salt promotes heart health, kidney health, vascular health, or a combination thereof in a subject. In some embodiments, the salt is iodized. In some embodiments, the salt is used for food preparation, seasoning, and/or preservation.

Some embodiments of the invention can be packaged as single-serving salt packets that consumers can use to season an individual meal or dish. Consumers with hypertension can use this embodiment to enjoy the seasoning benefits of salt while receiving a nutritionally effective amount of Br⁻. As a result, the invention enables the consumer to both enjoy their favorite foods and maintain a healthy diet

Some embodiments can be packaged and distributed in bulk quantities for food processing. By substituting this salt for normal table salt, food processors can supplement their products with nutritionally effective amounts of Br³¹ . These Br-enhanced products may have greater appeal to health-conscience consumers.

Sodium chloride is extensively used in food processing, such as some packaged foods and canned foods. Some foods are processed with reduced sodium, in effort to appeal to health-conscious consumers. However, some consumers can be unsatisfied with the taste of low-sodium or reduced-sodium products.

### Preparation of the Compositions

In embodiments, the compositions of the invention described herein can be manufactured through methods and procedures that are known to one skilled in the art of manufacturing saline solutions. For example, for manufacturing an embodiment of the invention comprising about 1 ml of about 100 μM Br⁻ and about 154 mM Cl⁻ in a prefilled syringe, about 7.99 mg of NaBr and about 9 g of NaCl can be dissolved in a total volume of about 1 L of water or aqueous solution. Then, 1 ml aliquots can be placed inside a syringe for storage and distribution. The water can be pharmaceutical grade, United States Pharmacopeia (USP) grade, or equivalent. The final solution can be sterilized through terminal sterilization or through the use of aseptic manufacturing methods.

Any pharmaceutically-acceptable salt of Br⁻ is suitable for manufacture of the invention. Non-limiting examples comprise sodium bromide, potassium bromide, and magnesium bromide. Compounds that contain a bromine atom as a covalently-bound substituent are not suitable for the invention since they are not salts.

In some embodiments, a composition of the invention is manufactured by adding a therapeutically effective amount of Br⁻ to a saline solution. For example, embodiments of the invention can be manufactured using a Br-deficient saline solution, with non-limiting examples comprising 0.9% NaCl, Ringer's solution, and Hartmann's solution. Some embodiments of the invention are manufactured by adding Br⁻ to a Br-deficient saline solution, where only an amount of Br⁻ needed to bring the final concentration of Br⁻ to an amount between about 30 μM and about 1 mM Br³¹ . For example, an embodiment of the invention comprising about 100 μM Br⁻ and about 0.9% NaCl can be manufactured by adding about 7.99 mg of NaBr to about 1 L of about 0.9% NaCl.

To ensure quality, various tests can be performed during the manufacturing. For example, mass spectrometry can be used to determine the amount of Br⁻ in the described compositions of the invention. As described herein, the element bromine is found as two isotopes with nearly equal proportion in nature, Br⁷⁹ and Br⁸¹, which assists in identifying Br⁻ by mass spectrometry. Other non-limiting examples of useful techniques for measuring Br⁻ include column chromatography, inductively coupled plasma mass spectrometry, x-ray florescence, and neutron activation analysis.

Embodiments of the invention are amenable to many various types of packaging. Non-limiting examples include pre-filled syringes, droppers, tubes, bags, vials, bottles, cans, capsules, inhalers, aerosol cans, or packets. Compositions of the invention described herein can be administered to a subject via a dropper, a syringe, a needle, a tube, a bag, a can, an aerosol can, a capsule, a bottle, a vial, or a packet. Alternatively, compositions of the invention described herein can be added to an individually-packaged bandage in order to moisten the bandage. For example, the moistened bandage may be applied to an open wound. Packaged forms of the compositions of the invention described herein are amenable to either room temperature storage or cold storage. Compositions of the invention described herein can be prescription drugs, over-the-counter drugs, or medical devices. Non-limiting examples of final product forms of these embodiments comprise pre-filled syringes, vials, aerosol cans, inhalers, bottles, and bags.

Compositions of the invention described herein are amenable to a variety, but not infinite, of formulations. Compositions can be formulated as a pill, a patch, a liquid, an injectable liquid, a powder, a lozenge, a food, a drink, a candy, or a gum. In some embodiments, compositions are not formulated for inhalation. In some embodiments, compositions of the invention described herein are not formulated with thiocyanate. Liquid embodiments, for example, are not formulated with less than about 0.1% sodium chloride. In embodiments, compositions of the invention described herein are not formulated as eye drops. Some compositions can be formulated for systemic administration to a subject, such as but not limited to injection or oral administration according to methods available to those skilled in the art.

Compositions of the invention described herein can be combined with another pharmaceutical drug into a combination product, where a synergistic effect can be observed. Non-limiting examples of pharmaceutical drug include manufacturing and packaging a combination drug product that comprises an embodiment of the invention and a drug for treating diabetes. Some embodiments are manufactured separately but combined with another pharmaceutical drug prior to administration to a subject, where a non-limited example is combining a liquid embodiment and a liquid diuretic for administration as a single treatment to a subject. Embodiments of the invention can be combined with another other drug listed herein.

Embodiments of the invention can be packed for single administration to a subject, such as not but limited to a single injection of an embodiment to a hospitalized subject. Some embodiments can be packaged for repeated dosing of a subject, such as but not limited to a pill bottle containing a multi-day supply of embodiments or a bubble strip containing a multi-day supply of embodiments.

Embodiments can be administered through one or more formulations and packaging formats. A non-limiting example includes administering embodiments as a daily pill to a subject, where the pill is self-administered at home, and administering injection embodiments to the same subject during regular office visits.

Some embodiments comprise administering doses of Br⁻ at differing concentrations over a period of time in order to achieve the desired circulating Br⁻ level in the blood, serum, or plasma of a subject. For example, a subject can be administered a composition comprising a high dose of Br⁻, such as but not limited to 15-25 mg Br⁻, followed by subsequent administration of lower doses of BC, such as but not limited to 1-5 mg Br⁻ a period time thereafter. This approach may particularly benefit a subject who has serum Br⁻ levels below 20 μM, where the initial large dose serves to restore serum Br⁻ levels to between about 50 μM and about 500 μM while the subsequently administered lower doses of Br⁻ serve to maintain the serum Br⁻ levels in this range. Another non-limiting example includes administering compositions comprising increasing doses of Br⁻ to a subject until a desired maintenance dosing is achieved, such as but not limited to increasing dosing from 1 mg Br⁻ to 3 mg Br⁻ to 5 mg Br⁻ and so on until a maintenance dose of 10 mg Br⁻ is achieved. Still another non-limiting example includes administering a daily maintenance dose of 5 mg Br⁻ with periodic injections of larger doses such as but not limited to 10 mg Br⁻, 15 mg Br⁻, 20 mg Br⁻, or 25 mg Br³¹ .

Importantly, in order to prevent toxicity, embodiments as described herein do not provide for administering sufficient amounts of Br⁻ to raise the circulating Br⁻ concentration above 1 mM in a subject, particularly if two or more separate compositions, each comprising Br—, are administered to the same subject. As a non-limiting example, if a subject is administered a combination of two or more drug formulations each containing Br—, for example a first drug for controlling diabetes combined with a second drug for controlling hypertension, it would be out of scope of the invention for the subject's blood concentration of Br⁻ to rise above 1 mM. Thus, embodiments as described herein comprise only administering sufficient Br⁻ to raise and maintain circulating Br⁻ concentration above at least 20 μM Br⁻ but not more than 1 mM. Further embodiments administer sufficient Br⁻ to achieve circulating Br⁻ concentrations above 50 μM but below 500 μM Br³¹ .

### EXAMPLES

### Example 1

Dialysis is known to place subjects into a chronic state of low Br⁻ levels. Depletion of Br⁻ is reported to occur after consumption of a high salt diet. However, the chronic effects of dietary salt on serum Br⁻ is not known. More importantly, it is not known whether salt-induced depletion of Br⁻ can cause disease in an individual.

To test whether Br⁻ can therapeutically treat cardiovascular disease and/or kidney disease, Br⁻ was administered to Dahl rats while on a high salt (8%) diet. Administration of high salt to Dahl rats is known to trigger hypertension, structural changes in the heart and vasculature, and kidney injury, thus modeling similar pathologic changes occurring in clinical cases of cardiovascular disease and/or kidney disease.

Dahl rats were administered a base diet of AIN76A with 8% NaCl, while a parallel arm was administered AIN76A with 8% NaCl and 300 mg of NaBr per kg of food. Mortality events were counted over the 10 week study. High salt treatment, without administration of Br⁻, resulted in a survival probability of only 44% while Br-treatment yielded a survival probability of 67%. Thus, administration of Br⁻ and Cl⁻ together increased the survival probability of 52% as compared to administration of Cl⁻ alone (FIG. 1), resulting in a synergistic effect.

### Example 2

Dahl rats are known to exhibit salt-sensitive hypertension and kidney disease. However, there is no data regarding the amount of Br⁻ in these rats after high salt administration. The inventors hypothesized that high dietary salt would result in the depletion of Br⁻ from these rats. Thus, Dahl rats were fed a diet supplemented with 8% NaCl (AIN76A base diet) over the course of 10 weeks. High salt diet depleted mean serum Br⁻ to 14.14 μM compared to 25.54 μM in control rats on normal salt diet (AIN76A) (FIGS. 1,2).

To test the invention, another group of Dahl rats were administered AIN76A supplemented with 8% NaCl and 300 mg NaBr per kg of food. Considering that the rat body weights increased from around 300 g to around 400 g over the course of the study, and assuming that the rats ate a relatively constant amount of 25 g of food per day, the daily consumption of Br⁻ ranged from approximately 14.6 to 19.4 mg Br⁻ per kg body weight, in accordance with the invention. Rats receiving Br⁻ and high salt displayed a mean serum Br⁻ of 246.50 μM at the end of the study (FIG. 2). Thus, the invention succeeded in providing a nutritionally effective amount of Br⁻ to the rats in the presence of a high salt diet.

### Example 3

Safety of the invention was demonstrated in spontaneously hypertensive rats (SHR). Rats were administered 10 mg of NaBr per kg of AIN76A diet for 10 weeks. At the end of the study, serum Br⁻ levels in Br-treated rats were compared to serum Br⁻ levels in SHR on AIN76A as well as Wistar rats on AIN76A. Administration of the invention resulted in a mean serum Br⁻ of 281.7 μM (±34.9 μM), while AIN76A-treated SHR displayed 33.1 μM (±5.4 μM) and Wistar rats on AIN76A diet displayed serum Br⁻ levels of 29.6 μM (±3.1 μM) (FIG. 2). The serum Br⁻ levels in the Br-treated rats are below the toxicity threshold of 12 mM serum Br⁻ in humans. Considering that the invention was administered for 10 weeks, it can be assumed that the end-of-study serum Br⁻ measurements reflect a steady-state level. Moreover, in the situation that the invention is administered to a patient being treated with dialysis, it can be further assumed that the Br⁻ administered through the invention would be removed from the patient would be removed at the next dialysis treatment. Thus, this data supports the position that the invention is reasonably safe for administration.

### Example 4

Br-deficient saline solutions are commercially available as wound washes, such as 0.9% NaCl. Br⁻ is not formulated into these salines, yet small amounts of Br⁻ may theoretically be present in the final product due to contamination of the NaCl reagent during manufacturing. To determine the actual amount of Br⁻ present in two commercially available wound washes, samples were obtained and analyzed by epiboron neutron activation analysis, revealing Br⁻ concentrations of 15.27 μM and 12.39 μM, respectively (FIG. 3). These results are similar to the contamination limit of 100 ppm Br⁻ in 0.9% NaCl (154 mM), as governed by USP monographs (United States Pharmacopeial Convention, “Sodium Chloride” monograph, 2002), where 100 ppm Br⁻ in 154 mM NaCl amounts to about 15 μM Br⁻. Thus, these commercially available wound washes only contain contaminating levels of Br⁻, which contrasts with saline embodiments of the invention that contain Br⁻ concentrations of at least 30 μM.

### Example 5

To validate the potential cardioprotective effects of Br⁻ observed in Dahl rats, the inventors turned to the spontaneously hypertensive rat (SHR) model of cardiovascular disease. Eight SHR animals received AIN76A diet for 10 weeks while 8 SHR animals received AIN76A supplemented with 10 mg NaBr/kg diet. No treatment-induced mortality or electrolyte disturbances were detected. After 1 day on study diet, urinary protein levels were significantly lower in rats receiving Br⁻, a difference that persisted through the end of the study (FIG. 5).

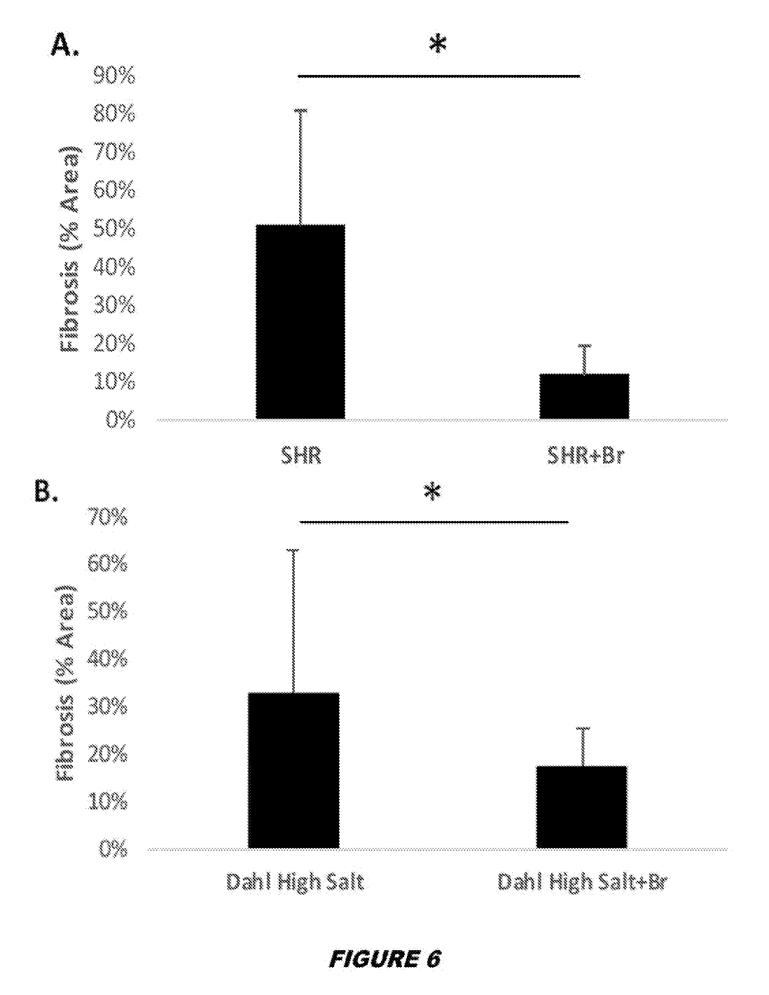

Following conclusion of the in vivo study, rat hearts were formalin-fixed, paraffin-embedded, and 5 μm-thick sections were obtained for histochemical analysis. Masson's Trichrome staining revealed dramatic reduction in blue staining in Br-treated hearts compared to SHR on AIN76A diet, suggesting that Br⁻ prevented the progression of fibrosis in cardiac tissue (FIG. 6).

### Example 6

To validate the potential cardioprotective effects of Br⁻ observed in Dahl rats, the inventors turned to the spontaneously hypertensive rat (SHR) model of cardiovascular disease. Eight SHR animals received AIN76A diet for 10 weeks while 8 SHR animals received AIN76A supplemented with 10 mg NaBr/kg diet. No treatment-induced mortality or electrolyte disturbances were detected. Br-treatment significantly reduced the absolute heart weight, and this trend was preserved when heart weight was adjusted to rat body weight (FIG. 5). After 1 day on study diet, urinary protein levels were significantly lower in rats receiving Br⁻, a difference that persisted through the end of the study (FIG. 5).

Following conclusion of the in vivo study, rat hearts were formalin-fixed, paraffin-embedded, and 5 μm-thick sections were obtained for histochemical analysis. Masson's Trichrome staining revealed dramatic reduction in blue staining in Br-treated hearts compared to SHR on AIN76A diet, suggesting that Br⁻ prevented the progression of fibrosis in cardiac tissue (FIG. 6).

### Example 7

The inventors next asked whether Br⁻ exerts any short-term activity in addition to the improved cardiac fibrosis noted after 10 weeks administration. Returning to the SHR preclinical model of disease, the inventors specifically asked whether Br⁻ administration modulates certain biomarkers associated with cardiovascular disease at one day post-administration. To this end, Br⁻ was administered to 8 SHR via intraperitoneal (IP) injection with an estimated 0.154 μg NaBr per gram body weight. For control, 8 SHR were administered saline via IP injection containing an estimated 4 ng NaBr per gram body weight. At one day post-administration, Br-treated rats exhibited statistically significant reductions in systolic blood (FIG. 7). Moreover, Br-treated rats displayed a statistically significant increase in serum brain natriuretic peptide (BNP; FIG. 8). Based on this data, the inventors conclude that practice of the invention as taught can therapeutically improve certain hemodynamic and circulating biomarkers that are associated with progression of cardiovascular and kidney diseases. Moreover, the inventors believe that Br⁻ achieves its cardiovascular and renal therapeutic activities in part via modulation of natriuretic peptide signaling.

### REFERENCES

The following references are hereby each incorporated by reference in its entirety.
Addison, Can. Med. Assoc. J, 18(3), 1928.
Akomeah et al., Eur. J. Pharm. Sci., 21(2-3):337, 2004.
Aouacheria et al., Mol. Biol. Evol., 23:2288, 2006.
Baird-Heinz, et al., J. Am. Vet. Med. Assoc., 2012
Bhave et al., Nat. Chem. Biol., 8(9):784, 2012.
Barratt and Walser, J. Clin. Invest., 48:56, 1969.
Baxter and Mitragotri, J. Control. Release, 106(3):361, 2005.
Bevc et al., Acta Dermatovenerol APA, 15(4):151, 2006
Borza et al., J. Biol. Chem., 280:27147, 2005.
Bramson et al., Gene Ther., 10(3):251, 2003.
Brodie et al., J. Biol. Chem., 130:555, 1939.
Canavese et al., Am. J. Kidney Dis., 48(6):1018, 2006.
Cao et al., Clin. Interv. Aging, 10:55,2015
Cheng et al., Free Radic. Biol. Med., 45:1682, 2008.
Chun and Uitto, Dermatol. Clin., 28(1):93, 2010.
Cope et al., J. American Chemical Soc., 82:4663, 1960.
Cortez et al., Arch. Biochem. Biophys., 254(2):504, 1987.
Cummings et al., J. Cell. Biol., 213(4):479, 2016.
Daley and Yamada, Curr. Opin. Genet. Dev., 23:408-414, 2013.
Davis et al., Biomaterials, 33(28):6691, 2012.
Dobbie et al., Adv. Peritoneal Dial., 6:3 1990.
Eli Lilly and Co. Hand Book of Pharmacy and Therapeutics, 1920.
Fidler et al., Proc. Natl. Acad. Sci. USA., 111(1):331, 2014.
Fox et al., Cell, 129:179, 2007.
Fu et al., J. Biol. Chem., 288(11):7430, 2013.
Gilchrist and Moody, Chemical Rev., 77:409, 1977.
Glass and Duchek, J. American Chemical Soc., 98:965, 1976.
Glibowicka et al., Biochem. Biophys. Acta., 946:345, 1988.
Gotenstein et al., Development, 137(21):3603, 2010.
Gould et al., N. Engl. J. Med., 354:1489, 2006.
Gould et al., Science, 308:1167, 2005.
Gupta et al., J. Cell. Biol., 137(5):1185, 1997.
Haenlein and Anke, Small Rumin. Res., 95:2, 2011.
Hasuike et al., Nephrol. Dial. Transplant., 19(6):1474, 2004.
He et al., J. Am. Soc. Nephrol., 27(4):1202, 2016.
Hudson et al., N. Engl. J. Med., 348:2543, 2003.
Hynes, Cell, 110:673, 2002.
Kay et al., J. American Chemical Soc., 114:10663, 1992.
Kennedy et al, Hypertension, 47:488-495, 2006.
Khan et al., Am. J. Hum. Genet., 89(3):464, 2011.
Khoshnoodi et al., J. Biol. Chem., 281:6058, 2006.
Khoshnoodi et al., Microsc. Res. Tech., 71:357, 2008.
Kivirikko and Pihlajaniemi, Adv. Enzymol. Relat. Areas Mol. Biol., 72:325, 1998.
Kleinewietfeld et al., Nature, 469(7446): 518, 2013.
Kubieck-Sutherland et al., EBioMedicine, 2(9):1169, 2015.
Kuo, et al., Hum. Mol. Genet., 21(R1):R97, 2012.
Lagerwerf et al., Rapid Commun. Mass Spectrom., 10:1905, 1996.
Lambeth and Swank, Federation Proc., 38:830, 1979.
Lee et al., Nephrol. Dial. Tranplant., 23:1005, 2008.
Licklider et al., Anal. Chem., 74:3076, 2002.
Lourenco et al., Eur. J. Heart Fail. 11(2), 2009.
Malara et al., Stem Cells, 32:926, 2014.
Marcinkiewicz et al., Adv. Exp. Med. Biol., 643:439, 2009.
Masago et al., Oncol. Lett., 2(2):253-247, 2011.
Matias et al., Port. J. Nephrol. Hypert., 23(2):161-166, 2009.
Matsue et al., J. Card. Fail., 21(11):859-864, 2015.
Maya and Villareal, J. Mol. Cell. Cardiol., 48(3):524-529, 2010.
McCall et al., Cell 157(6):1380, 2014.
Mertz, Science, 213:1332, 1981.
Miura et al., Nucl. Instr. and Meth. in Phys. Res. B, 189:443, 2002.
Moser et al., Science, 324:895, 2009.
Nambi et al., Eur. Heart J., 33:183, 2012.
Nathan et al., J. Am. Coll. Cardiol., 57(8):891-903, 2011.
Naughton, Am. Fam. Physician, 78(6):743-750, 2008.
Nelson et al., Embo J., 13:3438, 1994.
NICE Guideline, “Chronic kidney disease in adults: assessment and management (CG182), 2014
Ochi et al., Biol. Trace Elem. Res., 143:825, 2011.
Ogawa et al., Protein Sci
Palmer et al., J. Magnetic Resonance, 93:151, 1991.
Parkin et al., Hum. Mut., 32(2):127, 2011.
Pastor-Pareja and Xu, Dev. Cell, 21(2):245, 2011.
Pavelka et al., Physiol. Res., 54:639, 2005.
Pitt and Zannad, Circulation: Cardiovascular Imaging, 5:9-11, 2012.
Podell et al., J. Vet. Intern. Med., 7(5):318, 1993.
Poschl et. al., Development, 131(7):1619, 2004
Reid et al., J. Proteome Res., 3:751, 2004.
Reule & Drawz, Curr. Hypertens. Rep., 14(6), 2012.
Sackner-Bernstein et al., Circulation, 111(12):1487-1491, 2005
Sackner-Bernstein et al., JAMA, 293(15):1900-1905, 2005
Sakaguchi et al., Kidney International, 85:174, 2013.
Sakaguchi et al., PLoS One, 9(12):e116273, 2014.
Sawala, et al., Proc. Nat. Acad. Sci. U.S.A., 109:11222, 2012.
Schleucher et al., J. Biomol. NMR, 4:301, 1994.
Schlueter et al., Pharmacol. Ther., 144(1):12-27, 2014.
Serafino and Pierimarchi, Curr. Med. Chem., 21(21):2401-2409, 2014
Shroff et al., Circulation, 118:1748, 2008.
Siebold et al., Eur. J. Biochem., 176:617, 1988.
Senthilmohan and Kettle, Arch. Biochem. Biophys., 446(2): 235, 2006.
Song and Ott, Trends Mol. Med., 17(8):424, 2011.
Springer et al., J. Mol. Cell Cardiol., 52(5):1122, 2012
Strejan et al., Prog Clin Biol Res., 146:429, 1984.
Sundaramoorthy et al., J. Biol. Chem., 277:31142, 2002.
Tektas and Lutjen-Drecol, Exp. Eye Res., 88(4):769, 2009.
Than et al., Biological Chemistry, 386:759, 2005.
Than et al., Proc. Natl. Acad. Sci. USA, 99:6607, 2002.
Thorner et al., J. Biol. Chem., 271:13821, 1996.
Timpl et al., Eur. J. Biochem., 120(2):203, 1981.
Torii et al., Psychopharmacologia (Berl.), 29:65, 1973.
Trautner and Wieth, Acta. Physiol. Scand., 74:606, 1968.
Trepanier et al., J. Am. Vet. Med. Assoc., 213(10):1449, 1998
Tsuge et al., J. Health Sci., 46(5): 343, 2000.
Tuqan et al., Lasers Med. Sci., 20(2):80, 2005.
United States Pharmacopeial Convention, “Sodium Chloride” monograph, Pharmcopeial Forum, 28(4), July-August, 2002.
USRDS, Annual Data Report, 2013.
Van Leeuwen and Sangster, Toxicol., 18(3):189, 1987.
Van Renterghem et al., J. Trace Elem. Electrolytes Health Dis., 6:105, 1992.
Vanacore et al., J. Biol. Chem., 279:44723, 2004.
Vanacore et al., J. Biol. Chem., 280:29300, 2005.
Vanacore et al., J. Biol. Chem., 283:22737, 2008.
Vanacore et al., Science, 325(5945):1230, 2009.
Vesely, Anticancer Res., 34(4):1459-1466, 2014.
Vobecky et al., Biol. Trace Elem. Res., 103:37, 2005.
Vracko, Am. J. Pathol., 77:314, 1974
Wallaeys et al., Kidney Int., 30:599, 1986.
Wang et al., Nature, 455(7209):72-77, 2008.
Weber et al., Eur. J. Biochem., 139:401, 1984.
Weber et al., Nucl. Instr. and Meth. in Phys. Res. B3, 326, 1984.
Weil et al., Pediatrics, 80(4): 545-548, 1987.
Willker et al., Magnetic Resonance in Chemistry, 31:287, 1993.
Wolf and Eadie, Am. J. Physiol., 163:436, 1950.
Yap et al., Tissue Eng. Part A, 19(21,22):2361, 2013.
Yates et al., Anal. Chem., 67:1426, 1995.
Yamane et al., J. Neurochem., 1984
Yan et al., Hum. Mol. Genet., 23(21):5597, 2014.
Yanagisawa and Torii, Tohuko J. Exp. Med., 2002.
Yanagisawa and Yoshikawa, Biochem. Biophys. Acta, 329:283, 1973.
Yunos et al. Critical Care, 14:226, 2010.
Yurchenko, Cold Spring Harb. Perspect. Biol., 3(2):a004911, 2011
Yurchenco and Furthmayr, Biochemistry, 23:1839, 1984.

What is claimed is:

1. A pharmaceutical composition or a pharmaceutically acceptable salt thereof comprising an amount of Br⁻ and Cl⁻ ions, wherein the molar amount of Br⁻ ions is between about 0.0338% and about 4.613% of the molar amount of Cl⁻.

2. The composition of claim 1, wherein the Br⁻ and Cl⁻ ions are formulated as pharmaceutically acceptable salts of Na⁺, K⁺, Mg²⁺, Ca²⁺, or any combination thereof.

3. The composition of claim 1, wherein the composition is formulated as a pill, patch, strip, or liquid.

4. The composition of claim 1, wherein the composition is administered to a subject via oral, transdermal, injection, rectal, inhalation, nasal, or a combination thereof.

5. A method of treating or preventing a disease in a subject, the method comprising administering a therapeutically effective amount of a composition comprising an amount of Br⁻ and Cl⁻ ions wherein molar amount of Br⁻ ions is between about 0.0338% and about 4.613% of the molar amount of Cl⁻.

6. The method of claim 5, wherein the disease comprises cancer, cardiovascular disease, kidney disease, end stage renal disease, an eye disease, cystic fibrosis, a degenerative disease, a blood disorder, diabetes, a wound, a skin irritation, inflammation, an injury, a medical condition requiring dialysis treatment, or a combination thereof.

7. A pharmaceutical composition comprising a concentration of bromide ions between about 30 μM and about 1 mM, a concentration of chloride ions between about 17.1 mM and about 200 mM, and a biocompatible fluid.

8. The composition of claim 7, wherein the composition comprises a concentration of bromide ions (Br⁻) between about 50 μM and about 350 μM.

9. The composition of claim 7, wherein the composition comprises 0.9% sodium chloride (NaCl).

10. The composition of claim 7, wherein the composition is formulated to be used as a drug, a medical device, or as a replacement solution for renal replacement therapy.

11. A method of cleansing, washing, moistening, lubricating, harvesting, transporting, or storing a medical device, organ, tissue, or tissue product with a biocompatible fluid, the method comprising contacting a surface thereon in need of cleansing, washing, moistening, lubricating, harvesting, transporting, or storing with the composition of claim 7, wherein the composition of claim 7 cleanses, washes, hydrates, perfuses, moistens, lubricates, harvests, transports or stores the surface thereon.

12. A method of treating a subject in need thereof, the method comprising administering to the subject a therapeutically effective amount of the composition of claim 1 or claim 7 and administering a second therapeutic composition sequentially or concomitantly.

13. The method of claim 12, wherein the second composition comprises a pharmaceutical drug.

14. The method of claim 12, wherein the second composition is administered for the treatment of cancer, cardiovascular disease, kidney disease, end stage renal disease, an eye disease, cystic fibrosis, a degenerative disease, a blood disorder, diabetes, metabolic syndrome, obesity, a wound, a skin irritation, inflammation, an injury, a medical condition requiring dialysis treatment, or a combination thereof.

15. The method of claim 12, wherein the route of administration of the composition of claim 1 and the second composition comprises injection, inhalation, topically, orally, or a combination thereof.

16. The method of claim 12, wherein the subject is afflicted with cancer, cardiovascular disease, kidney disease, an eye disease, a degenerative disease, cystic fibrosis, a blood disorder, diabetes, a wound, a skin irritation, inflammation, an injury, a medical condition requiring dialysis treatment, a chronic wound, dermal ulcer, pressure ulcer, mouth sore, diabetic ulcer, sepsis, fluid loss, acid-base imbalance, electrolyte imbalance, diabetes, metabolic syndrome, obesity, blood cancer, solid tumor, recurrent cancer, metastatic tumor, a medical condition caused by an operation, an infection, trauma, natural aging process, or a combination thereof.

17. A pharmaceutical composition comprising a concentration of bromide ions between about 30 μM and about 1 mM, a concentration of chloride ions between about 17.1 mM and about 200 mM, a concentration of magnesium ions (Mg²⁺) between about 0.75 mM and about 3 mM, and a biocompatible fluid.

18. The composition of claim 17, wherein the composition comprises about 0 mEq/L to about 35 mEq/L lactate, about 1 mEq/L to about 5 mEq/L calcium, about 22 mEq/L to about 32 mEq/L bicarbonate (HCO₃⁻), about 0 mEq/L to about 4 mEq/L potassium, about 140 mEq/L sodium, about 0 mEq/L to about 1 mEq/L hydrogen phosphate (HPO₄²⁻), about 0 mEq/L to about 100 mEq/L dextrose, or a combination thereof.

19. The composition of claim 17, wherein the composition further comprises a therapeutically effective amount of bicarbonate, potassium, sodium, calcium, glucose, dextrose, acetate, citric acid, or any combination thereof.

20. The composition of claim 17, wherein the composition is used as a dialysate to perform dialysis on a subject.

21. A method of preventing or treating disease in a subject, the method comprising performing dialysis on the subject against a dialysis fluid comprising a sufficient amount of Br⁻ to maintain the subject's serum Br⁻ between about 30 μM and about 1 mM and a sufficient amount of Mg²⁺ to maintain the subject's serum Mg²⁺ between about 2.5 mg/dl and about 3.0 mg/dl.

22. The method of claim 21, wherein the disease comprises cardiovascular disease.

23. A nutritional composition for promoting heart health, kidney health, vascular health, or a combination thereof in a subject, the composition comprising between about 0.1 mg and about 50 mg Br³¹ .

24. The composition of claim 23, where the composition further comprises magnesium, vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid), vitamin B₁₂ (cobalamin), vitamin C (ascorbic acid), vitamin E (tocopherol), selenium, zinc, iron, vitamin D, or a combination thereof.

25. The composition of claim 23, wherein the composition comprises less than 50 mg taurine.

26. The composition of claim 23, where the composition is orally administered to the subject.

27. The composition of claim 23, where the subject has cardiovascular disease, hypertension, kidney disease, end stage renal disease, diabetes, metabolic syndrome, obesity, cancer, an eye disease, an ear disease, a wound, an ulcer, an infection, inflammation, a degenerative disease, a skin disease, cystic fibrosis, or a combination thereof.

28. A table salt composition comprising Br⁻ and Cl⁻ ions, wherein the mass of Br⁻ ions are between about 0.0338% and about 4.613% of the mass of Cl⁻.

29. The composition of claim 28, where the salt is iodized.

30. The composition of claim 28, where the salt is used for food preparation, seasoning, and/or preservation.

31. A method of preventing cardiovascular disease or kidney disease in a subject, the method comprising administering to the subject a nutritiously effective amount of a dietary item containing a molar amount of Br⁻ ions between about 0.0338% and about 4.613% of the molar amount of Cl⁻.

32. The method of claim 31, wherein the circulating Br⁻ level in the serum, blood, or plasma of the subject is at or below 20 μM.

33. The method of claim 31, where administration of the dietary item maintains circulating Br⁻ levels in the subject to above about 50 μM and below about 500 μM.

34. A method of manufacturing a dietary item to promote heart health, kidney health, vascular health, or a combination thereof, the method comprising adding a nutritionally effective amount of bromide (Br⁻) to the dietary item.

35. The method of claim 34, where the nutritiously effective amount of Br⁻ comprises between about 0.1 mg and about 50 mg of Br³¹ .

36. The method of claim 34, wherein the dietary item comprises a food product, dietary ingredient, medical food, drug, functional food, beverage, dietary supplement, vitamin, mineral, or combination thereof.

37. A method of promoting heart health, kidney health, vasculature health, or a combination thereof in a subject, the method comprising:
Identifying a subject in need thereof, wherein the subject displays at least one of:
i. Circulating Br⁻ level at or below 20 μM, as measured in serum, blood, or plasma;
ii. Systolic blood pressure about 120 mm Hg or higher;
iii. Diastolic blood pressure at about 80 mm Hg or higher;
iv. Resting heart rate about 100 beats per minute or higher;
v. GFR about 80 ml/min/1.73 m2 or lower;
vi. Urinary albumin level about 30 mg/g or higher;
Obtaining a dietary item or drug comprising an amount of Br— between about 0.1 mg and about 50 mg; and
Administering an effective amount of the dietary item or drug required to raise or maintain the circulating levels of Br⁻ in the subject's blood, serum, or plasma between about 50 μM and about 1 mM.

38. The method of claim 37 further comprising monitoring the safety of thedietary item or drug by measuring the amount of Br⁻ in the subject's blood, serum, or plasma.

39. The method of claim 37 further comprising ceasing administration of the dietary item or drug if the blood, serum, or plasma Br⁻ level approaches 1 mM

40. The method of claim 37, the method comprising administering a dietary item or drug that maintains the circulating Br⁻ levels in the subject to between about 50 μM and about 500 μM.
